(19) United States
(12) Patent Application Publication
(10) Pub. No.: US 2025/0369020 A1
(43) Pub. Date: Dec. 4, 2025

(54) PRODUCTION AND/OR DELIVERY OF MULTISPECIFIC BINDING AGENTS

(71) Applicant: Seattle Children's Hospital (dba Seattle Children's Research Institute), Seattle, WA (US)

(72) Inventors: David J. Rawlings, Seattle, WA (US); Richard G. James, Seattle, WA (US)

(21) Appl. No.: 18/864,832

(22) PCT Filed: May 9, 2023
(86) PCT No.: PCT/US2023/066760

§ 371 Date: Nov. 11, 2024

Related U.S. Application Data
(60) Provisional application No. 63/343,537, filed on May 18, 2022.
(60) Provisional application No. 63/498,202, filed on Apr. 25, 2023.

(51) Int. Cl. C12N 15/90; A61K 40/13; A61K 40/33; A61K 40/42; A61P 35/02; C07K 16/28; C12N 9/22; C12N 15/11

(52) U.S. Cl. CPC C12N 15/907; A61K 40/13; A61K 40/33; A61K 40/421; A61K 40/4211; A61P 35/02; C07K 16/2803; C12N 9/226; C12N 15/11; C07K 2317/31; C12N 2310/20; C12N 2800/80

(57) ABSTRACT

Some embodiments of the method and compositions provided herein relate to methods of preparing cells expressing bispecific T cell engagers (BTCEs), and the use of such cells in certain therapies. In some embodiments, the cells are B cells or B cell precursors.

### CROSS-REFERENCE TO RELATED APPLICATIONS

This application claims priority to U.S. Prov. App. No. 63/498,202 filed Apr. 25, 2023 entitled “PRODUCTION AND/OR DELIVERY OF MULTISPECIFIC BINDING AGENTS” and to U.S. Prov. App. No. 63/343,537 filed May 18, 2022 entitled “PRODUCTION AND/OR DELIVERY OF MULTISPECIFIC BINDING AGENTS” which are each incorporated by reference herein in its entirety.

### REFERENCE TO SEQUENCE LISTING

The present application is being filed along with a Sequence Listing in electronic format. The Sequence Listing is provided as a file entitled SCRI416WOSEQLIST, created May 8, 2023, which is approximately 97,869 bytes in size. The information in the electronic format of the Sequence Listing is incorporated herein by reference in its entirety.

### FIELD OF THE INVENTION

Some embodiments of the method and compositions provided herein relate to methods of preparing cells expressing bispecific T cell engagers (BTCEs), and the use of such cells in certain therapies. In some embodiments, the cells are B cells or B cell precursors.

### BACKGROUND OF THE INVENTION

Bispecific antibodies target two different epitopes, often on two different antigens, and have been described as “a key component of the next generation of antibody therapy”. See, for example, Wang et al., Antibodies 8:43, 2019. Various formats of bispecific antibodies have been developed. Commercial development of bispecific antibodies has been described as having been “hampered” by “great production challenges”.

### SUMMARY OF THE INVENTION

Some embodiments of the methods and compositions provided herein include a system for modifying a cell to express a bispecific T-cell engager (BTCE), comprising: (a) a nuclease or nucleic acid encoding the nuclease capable of inserting an expression cassette into a locus in a cell genome; and (b) a first polynucleotide encoding a homology direct repair (HDR) template comprising the expression cassette, wherein the expression cassette encodes the BTCE.

Some embodiments also include the cell, wherein the cell is a B cell or a B cell precursor. In some embodiments, the cell is selected from a hematopoietic stem cell, a human embryonic stem cell, an induced pluripotent stem cell (iPSC), a naïve B cell, a memory B cell, a plasmablast, or a plasma cell.

In some embodiments, the nuclease is a Cas nuclease; and optionally, wherein the nuclease is a Cas9 nuclease.

Some embodiments also include a second polynucleotide encoding a guide RNA (gRNA). In some embodiments, the second polynucleotide comprises a DNA or RNA sequence corresponding to a nucleotide sequence of any one of SEQ ID NOs: 01-27.

In some embodiments, the locus comprises an endogenous gene expressed at a higher level in a B cell than in a non-B cell. In some embodiments, the locus comprises an endogenous gene inactive in a B cell. In some embodiments, the locus is selected from a CCR5 gene, a JCHAIN gene, the IGHM locus (also known as E-mu; hg38 genome; chr14; 105856225-105863200), a CD19 gene, or an IGHG1 gene.

In some embodiments, the BTCE comprises a first polypeptide capable of specifically binding to a T-cell antigen, and a second polypeptide capable of specifically binding to a tumor antigen. In some embodiments, the T-cell antigen comprises CD3. In some embodiments, the tumor antigen is selected from CD33, CD19, CD326 (EpCAM), neuron-glial antigen 2 (NG2), HER2, epidermal growth factor receptor (EGFR), CD66e, ephrin type-A receptor 2 (EphA2), CD21, FLT3, gp100, PDL1, or CD22. In some embodiments, the BTCE comprises AMG 330, blinatumomab, solitomab, or tebentafusp.

In some embodiments, the expression cassette further comprises a promoter. In some embodiments, the promoter is a constitutive promoter. In some embodiments, the promoter is an inducible promoter. In some embodiments, the promoter is an MND promoter, an IgVH promoter, an EF-1α promoter, or an IgHG1 promoter.

In some embodiments, the expression cassette also includes: (i) an enhancer, optionally wherein the enhancer comprises an Eμ enhancer, or an SLC3A2 enhancer; (ii) a polynucleotide encoding a signal sequence, optionally wherein the signal sequence is an IgHV signal sequence or an IgHG1 signal sequence; (iii) a polynucleotide comprising a 5′ UTR from an IGHV gene or an IGHG1 gene; (iv) a polynucleotide comprising a 3′ UTR from an IGHV gene or an IGHG1 gene; and/or (v) a ubiquitous chromatin-opening element (UCOE).

In some embodiments, the first polynucleotide further comprises a nucleic acid homologous to the locus; and optionally, wherein the nucleic acid homologous to the locus has a length in a range from about 200 to about 1500 consecutive nucleotides.

In some embodiments, a vector comprises the first polynucleotide. In some embodiments, the vector comprises a viral vector. In some embodiments, the viral vector is an adeno associate viral (AAV) vector, or a lentiviral vector. In some embodiments, the AAV vector is an AAV6 vector.

In some embodiments, the cell is mammalian; and optionally, wherein the cell is human. In some embodiments, the cell is ex vivo. In some embodiments, the cell is autologous to a subject. In some embodiments, the cell is allogeneic to a subject. In some embodiments, the cell lacks expression of an endogenous protein to which the BTCE is capable of specifically binding.

Some embodiments include a method for modifying a cell to express a bispecific T-cell engager (BTCE), comprising: (a) obtaining the system of any one of claims 2-28; and (b) introducing into the cell the nuclease or nucleic acid encoding the nuclease and the first polynucleotide to obtain a modified cell.

Some embodiments also include activating the cell prior to step (b); and optionally, wherein the activating comprises contacting the cell with an oligomerized CD40 ligand (CD40L), CpG, and/or IL-21.

In some embodiments, step (b) comprises contacting the cell with a ribonucleoprotein (RNP) comprising the nuclease and the gRNA.

Some embodiments also include (c) differentiating the cell.

Some embodiments include a cell modified by any one of the foregoing methods.

Some embodiments include a cell, wherein the cell is genetically modified at a genomic locus to express a bispecific T-cell engager (BTCE).

In some embodiments, the cell is a B cell or a B cell precursor. In some embodiments, the cell is selected from a hematopoietic stem cell, a human embryonic stem cell, an induced pluripotent stem cell (iPSC), a naïve B cell, a memory B cell, a plasmablast, or a plasma cell.

In some embodiments, the locus comprises an endogenous gene expressed at a higher level in a B cell than in a non-B cell. In some embodiments, the locus comprises an endogenous gene inactive in a B cell. In some embodiments, the locus is selected from a CCR5 gene, a JCHAIN gene, the IGHM locus (also known as E-mu; hg38 genome; chr14: 105856225-105863200), a CD19 gene, or an IGHG1 gene.

In some embodiments, the BTCE comprises a first polypeptide capable of specifically binding to a T-cell antigen, and a second polypeptide capable of specifically binding to a tumor antigen. In some embodiments, the T-cell antigen comprises CD3. In some embodiments, the tumor antigen is selected from CD33, CD19, CD326 (EpCAM), neuron-glial antigen 2 (NG2), HER2, epidermal growth factor receptor (EGFR), CD66e, ephrin type-A receptor 2 (EphA2), CD21, FLT3, gp100, PDL1, or CD22. In some embodiments, the BTCE comprises AMG 330, blinatumomab, solitomab, or tebentafusp.

In some embodiments, the genomic locus is modified by insertion of an expression cassette comprising a first nucleic acid encoding the BTCE.

In some embodiments, the first nucleic acid is operably linked to an endogenous promoter at the locus. In some embodiments, the expression cassette further comprises a promoter operably linked to the first nucleic acid. In some embodiments, the promoter is a constitutive promoter. In some embodiments, the promoter is an inducible promoter. In some embodiments, the promoter is an MND promoter, an IgVH promoter, an EF-1α promoter, or an IgHG1 promoter.

In some embodiments, the expression cassette further comprises: (i) an enhancer, optionally wherein the enhancer comprises an Eμ enhancer, or an SLC3A2 enhancer; (ii) a polynucleotide encoding a signal sequence, optionally wherein the signal sequence is an IgHV signal sequence or an IgHG1 signal sequence; (iii) a polynucleotide comprising a 5′ UTR from an IGHV gene or an IGHG1 gene; (iv) a polynucleotide comprising a 3′ UTR from an IGHV gene or an IGHG1 gene; and/or (v) a ubiquitous chromatin-opening element (UCOE).

In some embodiments, the cell is mammalian; and optionally, wherein the cell is human. In some embodiments, the cell is ex vivo. In some embodiments, the cell is autologous to a subject. In some embodiments, the cell is allogeneic to a subject.

Some embodiments include a pharmaceutical composition comprising any one of the foregoing cells.

Some embodiments include a method for treating, ameliorating or inhibiting a disorder in a subject, comprising: administering to the subject the cell of any one of claims 33-54. In some embodiments, the cell is administered in no more than a single dose; and optionally, wherein the cell is administered in a single bolus. In some embodiments, the disorder comprises a cancer. In some embodiments, the BTCE is capable of specifically binding to a tumor antigen expressed by the cancer. In some embodiments, the cancer is selected from a solid tumor and a leukemia. In some embodiments, the cancer is selected from acute lymphoblastic leukemia (ALL), acute myeloid leukemia (AML), breast cancer, stomach cancer, melanoma, colon cancer, colorectal cancer, head and neck cancer, gastric cancer, prostate cancer, ovarian cancer, lung cancer, and pancreatic cancer. In some embodiments, the subject is human.

Some embodiments include use of any one of the foregoing cells as a medicament such as a medicament for treating, ameliorating or inhibiting a disorder or a disease in a subject.

Some embodiments include use of any one of the foregoing cells in the preparation of a medicament for treating, ameliorating or inhibiting a disorder in a subject.

In some embodiments, the cell is administered in no more than a single dose; and optionally, wherein the cell is administered in a single bolus. In some embodiments, the disorder comprises a cancer. In some embodiments, the BTCE is capable of specifically binding to a tumor antigen expressed by the cancer. In some embodiments, the cancer is selected from a solid tumor and a leukemia. In some embodiments, the cancer is selected from acute lymphoblastic leukemia (ALL), acute myeloid leukemia (AML), breast cancer, stomach cancer, melanoma, colon cancer, colorectal cancer, head and neck cancer, gastric cancer, prostate cancer, ovarian cancer, lung cancer, and pancreatic cancer. In some embodiments, the subject is human.

### BRIEF DESCRIPTION OF THE DRAWINGS

FIG. 1A depicts photomicrographs for a characterization of NSG-huCD34 mice with hematoxylin and eosin staining of spleen sections from NSG and NSG-huCD34.

FIG. 1B depicts FACS analyses for spleen flow cytometry from NSGhuCD34 Spleen.

FIG. 1C depicts graphs for plasma cell cytokines measured in peripheral sera of NSG-huCD34 and NSG.

FIG. 2A depicts an in vitro timeline used to generate gene edited plasma cells that express luciferase derived from primary human B cells.

FIG. 2B depicts a graph for frequency of HDR edited alleles in primary B cells edited with and without luciferase vector.

FIG. 2C depicts a FACS analysis for plasma cell staining (CD138+CD38+) of live cells 11 days after gene-editing.

FIG. 2D depicts a graphs for IgG flow cytometry staining of plasma cells.

FIG. 3A depicts a timeline for in vivo studies in which NSG and NSG-huCD34 mice received gene-edited plasma cells (PCs) or PBS control.

FIG. 3B depicts luminescence images of NSG and NSG-huCD34 mice engrafted with bulk plasma cells show engraftment of ffluc-PCs.

FIG. 3C depicts graphs for average radiance of NSG and NSG-huCD34 mice engrafted with ffluc-PCs show improved engraftment and persistence in humanized mice.

FIG. 3D depicts photomicrographs for immunohistology of spleen from NSG-huCD34 with ffLuc-PCs. Arrows indicate ffLuc+CD138+ cells.

FIG. 3E depicts graphs for serum IgG titers in NSG and NSG-huCD34 mice that received bulk plasma cells.

FIG. 3F depicts flow plots of intracellular IgM and IgG of plasma cells (CD138+, CD38+,hCD45+) show IgG+ cells in mice that receive edited PCs.

FIG. 3G depicts FACS analyses for PCs edited to express BFP detected in the bone marrow of NSG-huCD34 mice.

FIG. 3H depicts a graph for percentage IgG+ plasma cells for bulk and enriched cells.

FIG. 3I depicts a graph for IgG concentration over time.

FIG. 4A depicts a schematic for a cassette used to generate BiTE secreting plasma cells.

FIG. 4B depicts representative FACS analyses for edited PCs.

FIG. 4C depicts a FACS analysis for activated T cells.

FIG. 4D depicts graphs for killing of K562-CD19+ cells in the presence of BiTE-PC (BTCE-PC) or BFP-PCs supernatant.

FIG. 5A depicts a schematic of an IGHG1 genomic locus and shows exon 1, 2, 3 and 4 (black boxes). Target locations for sgRNAs are shown and include TH16, TH22, TH27, TH28 in exon 1; TH26 in exon 2; and TH21 in exon 4.

FIG. 5B depicts a FACS analysis. The top panels depict a ˜90% decrease in IGHG+ cells in B cells observed in cells subjected to treatment with RNPs containing TH26 sgRNA. The bottom panels depict the GFP+ percentage in B cells engineered to express GFP at the IGHG1 locus.

FIG. 6A depicts a schematic of an Eμ genomic locus with an insertion site for expression cassettes, and schematics of example vectors containing expression cassettes. The expression cassettes include all or some of the following elements: a 5′ homology arm (HA) about 450 nucleotides in length; an IgVH promoter (IgVH_P); a nucleic acid encoding an anti-CD3 polypeptide portion of a BiTE («CD3); a nucleic acid encoding a linker (G4S); a nucleic acid encoding an anti-CD19 polypeptide portion of a BiTE (αCD19); a nucleic acid encoding an anti-CD33 polypeptide portion of a BiTE (αCD33); a P2A ribosome skip sequence (2A); a nucleic acid encoding a green florescent protein (GFP); a woodchuck hepatitis virus post-transcriptional regulatory element (WPRE); and a 3′ homology arm (HA) about 450 nucleotides in length.

FIG. 6B depicts a graph for percentage of live cells expressing GFP for constructs of FIG. 6A. (1) first column, cells edited with the expression cassette containing a GFP reporter gene only. (2) second column, cells edited with the expression cassette containing the nucleic acid encoding an anti-CD19 polypeptide portion of a BiTE. (3) third column, cells edited with the expression cassette containing the nucleic acid encoding an anti-CD33 polypeptide portion of a BiTE. (4) fourth column, unedited cells.

FIG. 7 depicts graphs for percentage activated T cells (left panel), percentage monocytes of live cells (middle panel), and percentage B cells of live cells (right panel). Left column of each graph relate to cells edited with the expression cassette containing the nucleic acid encoding an anti-CD19 polypeptide portion of a BiTE (CD19BITE); middle column of each graph relate to cells edited with the expression cassette containing the nucleic acid encoding an anti-CD33 polypeptide portion of a BITE (CD33BITE); and right column of each graph relate to cells edited with the expression cassette containing a GFP reporter gene only (GFP).

FIG. 8A depicts a schematic of example vectors containing expression cassettes. Constructs 1 and 2 had homology arms for delivery to the CCR5 locus. Constructs 3 and 4 had homology arms for delivery to the Eμ locus. Constructs 2 and 4 were negative controls that expressed fluorescent markers (BFP in construct 2, GFP in construct 4) downstream of an MND promoter. Constructs 1 and 3 expressed the Blinatumomab BiTE in cis with GFP, also downstream of an MND promoter.

FIG. 8B depicts a schematic of a CCR5 genomic locus with insertion site in exon 3 for construct (1) and (2).

FIG. 8C depicts a FACS analysis for cells edited at the CCR5 locus or Ep (Mu) locus with construct (1), (2), (3), or (4). The highlighted boxes indicate the expression of the fluorescent reporters in the absence of blinatumobab.

FIG. 8D depicts the FACS analysis shown in FIG. 8C in which the highlighted boxes indicate expression of the fluorescent reporter as expressed downstream of the blinatumomab.

FIG. 8E depicts a FACS analysis in which the allele frequency of HDR events for the BFP templates were similar to the proportion observed when quantifying BFP+ cells.

FIG. 9A depicts a schematic for a method used to quantify secreted blinatumomab.

FIG. 9B depicts a FACS analysis for cells incubated with either a high concentration of a BiTE, a high concentration of the BiTE, or no BiTE.

FIG. 9C depicts a graph of ratio of CD19+ cells to CD19-targets for various BiTE concentrations for two replicates.

FIG. 9D depicts a FACS analysis for cells incubated with either a high concentration of a BiTE, a high concentration of the BITE, or no BiTE.

FIG. 9E depicts a graph of percentage activated T cells for various BITE concentrations for two replicates.

˜8.

FIG. 9F depicts a graph of percentage activated T cells for various BiTE concentrations for two replicates.

FIG. 9G depicts a bar graph of percentage activated T cells for cells edited with a CCR5_BFP (first 2 columns at left), CCR5_BITE (second 2 columns), Mu_GFP (third 2 columns), and Mu BiTE (fourth 2 columns at right), each for two replicates.

FIG. 10A depicts a schematic of HDR templates cloned into AAV vector for the expression of GFP or blinatumomab_2A GFP at 4 different loci.

FIG. 10B depicts a schematic of a CCR5 genomic locus with insertion site in exon 3 for constructs (1) and (2).

FIG. 10C depicts a FACS analysis for HDR levels in cells edited with constructs.

FIG. 10D depicts a FACS analysis for HDR levels in cells edited with constructs.

FIG. 11A depicts a FACS analysis in B cells edited to express GFP and/or a BITE, and levels of CD19 and CD38 in the engineered cell population were quantified.

FIG. 11B depicts a bar graph of percentage T cell activation for cells edited with constructs: BFP construct (1st column), BFP CD19KO (2nd column), CCR5 BITE (3rd column), CCR5 BITE CD19KO (4th column), MuE BITE (5th column), and MuE BITE CD19KO (6th column).

FIG. 12A depicts an assay in which B cells were genetically modified to express blinatumomab (test) or GFP (control). Modified B cells were mixed with autologous T cells, and Raji cells (CD19+ B cell lymphoma), which had been modified to express luciferase. The cell mixture was implanted into the flank of immunodeficient mice (NSG).

FIG. 12B depicts a photograph of luminescence (blue circle in mice) with an IVIS instrument.

FIG. 12C depicts a photograph of luminescence (blue circle in mice) with an IVIS instrument.

FIGS. 13A-13K depict genome engineered primary human B cells secreted functional αCD19-BITE in a locus dependent manner. FIG. 13A depicts a schematic in which primary B cells were edited to express either BFP or αCD19BITE.T2A.GFP transgenes at CCR5 genetic loci via HDR using CRISPR Cas9 and AAV6 delivered DNA repair templates.

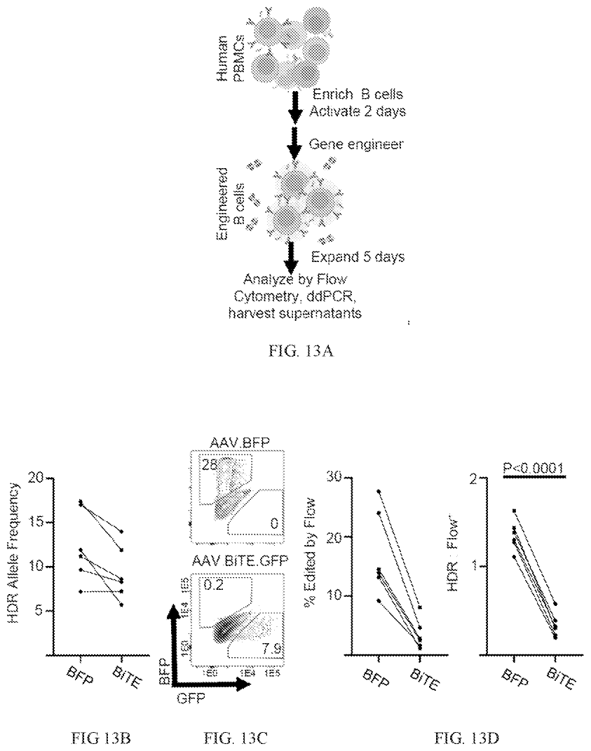

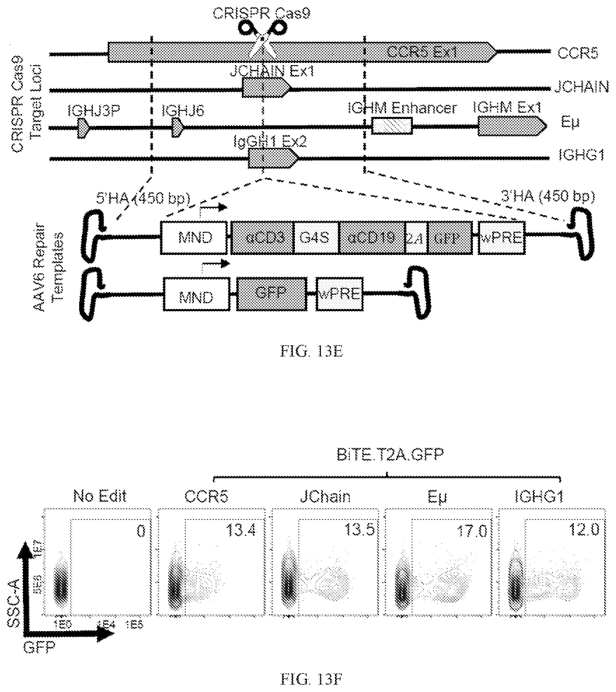

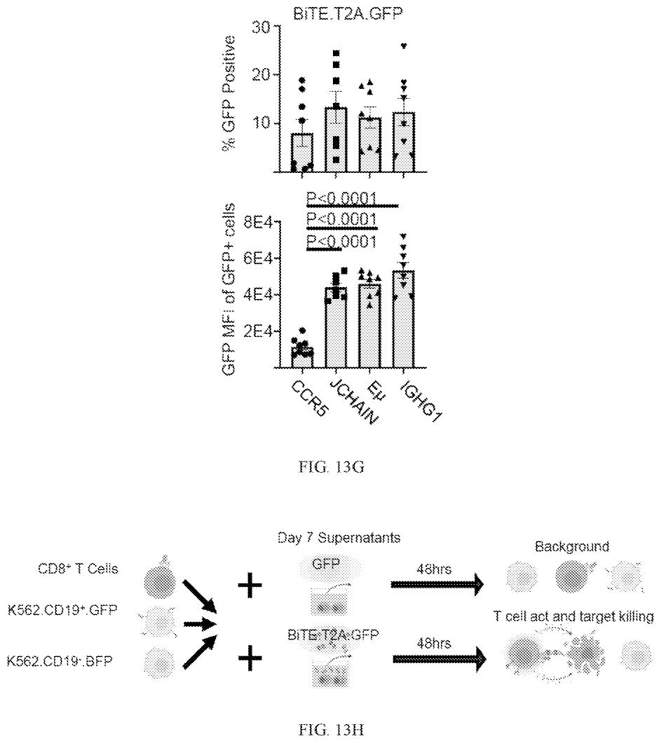

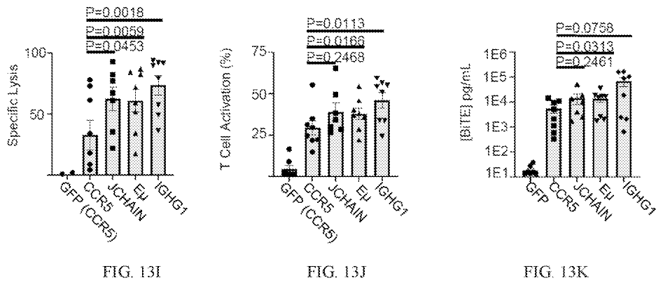

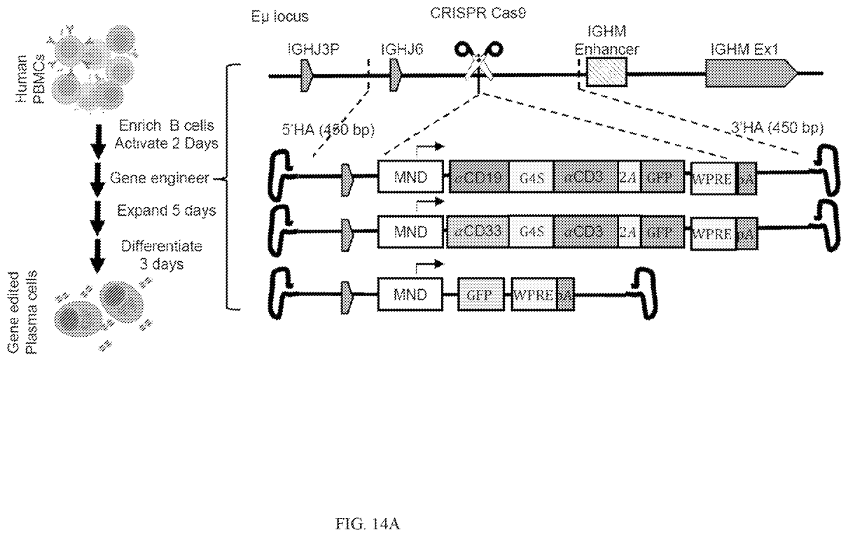

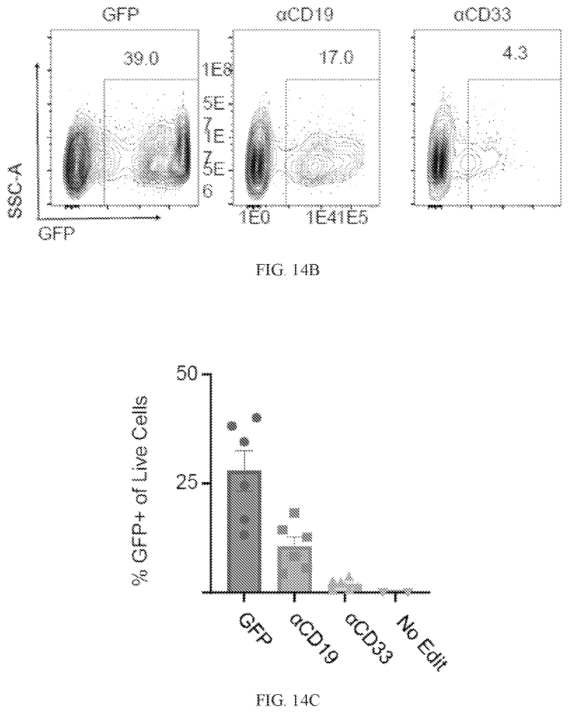

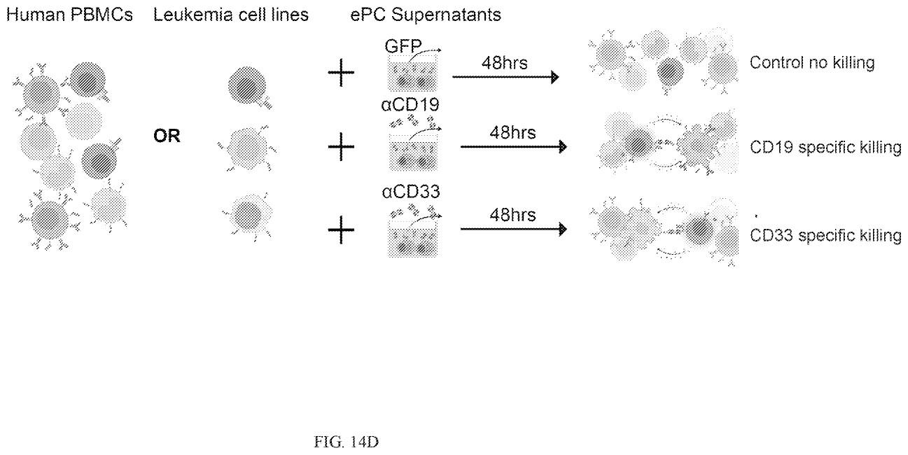

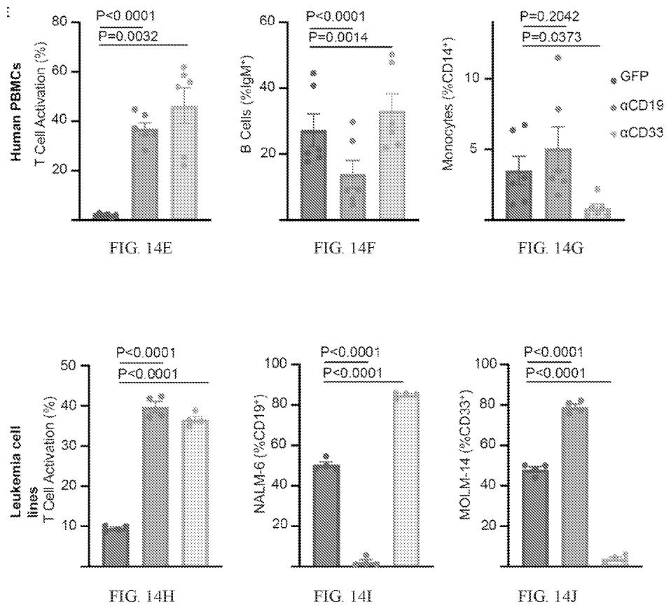

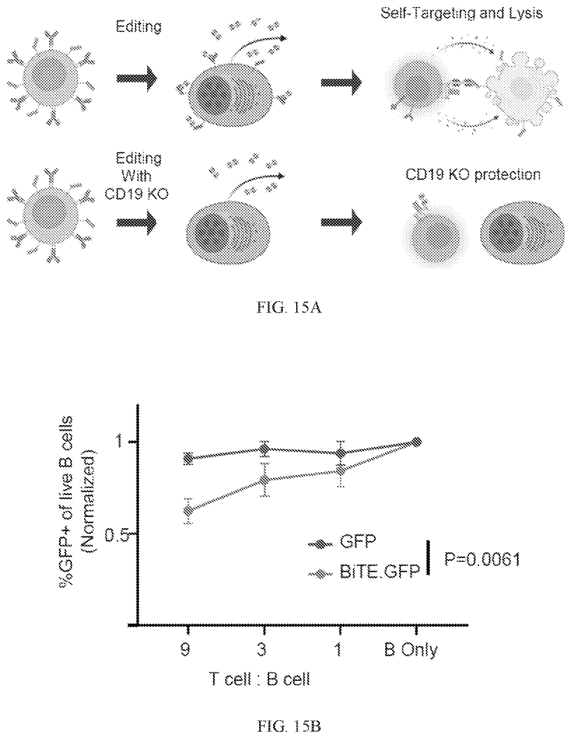

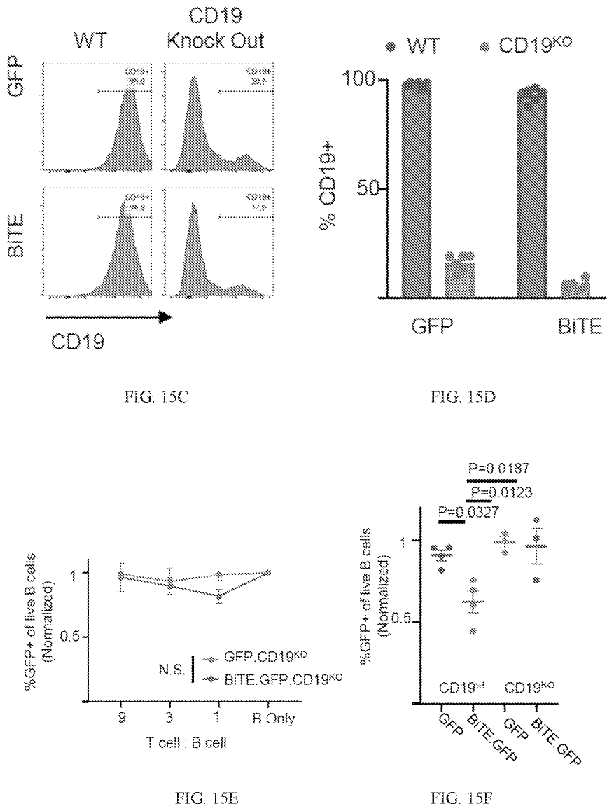

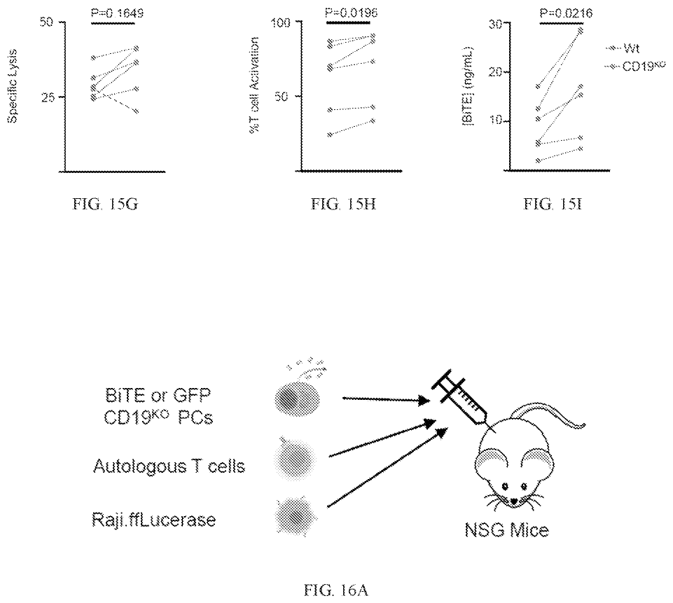

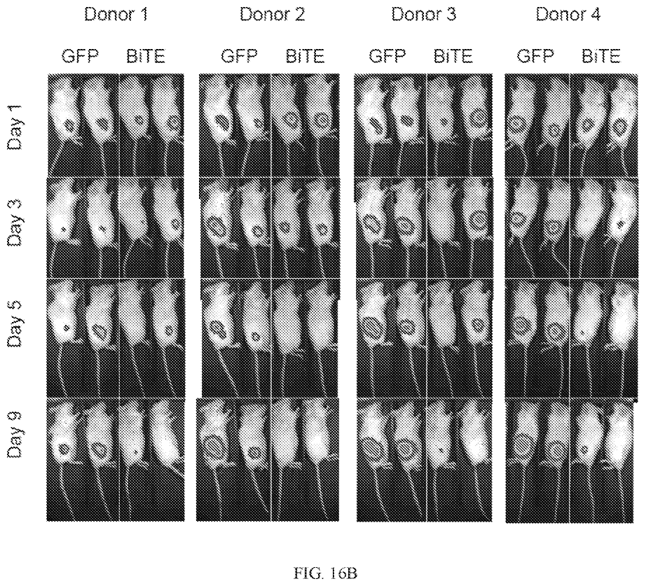

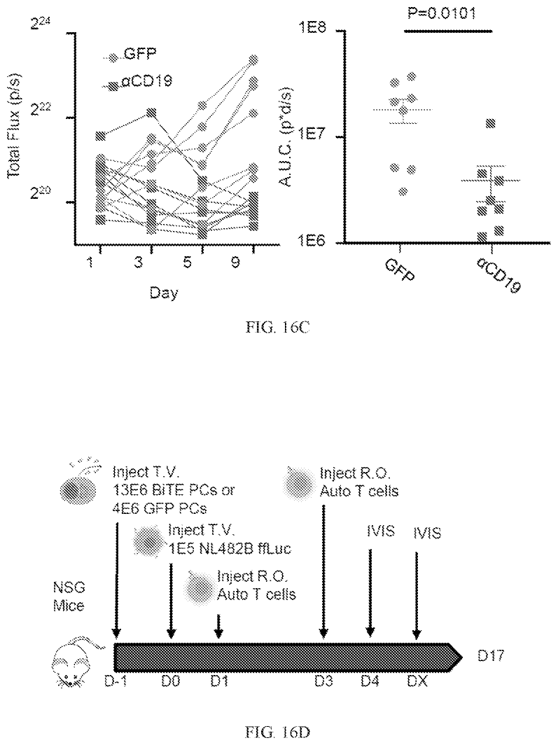

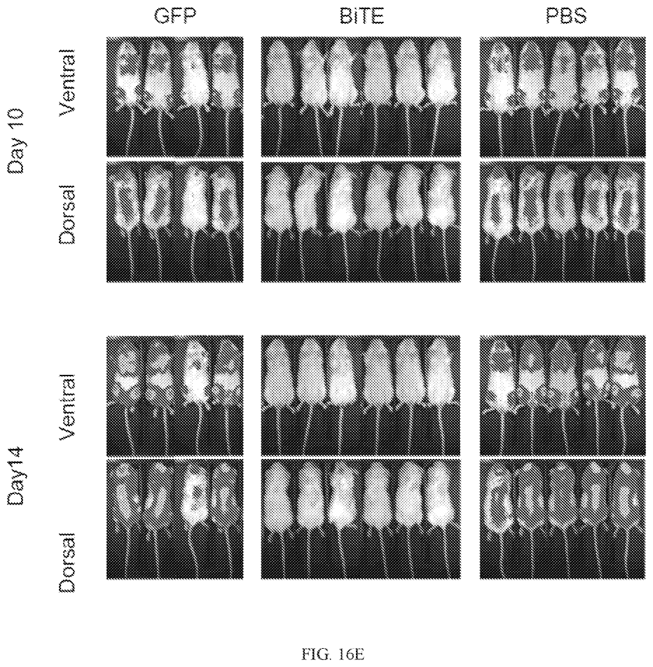

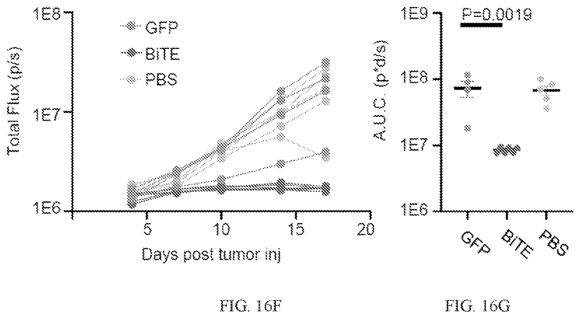

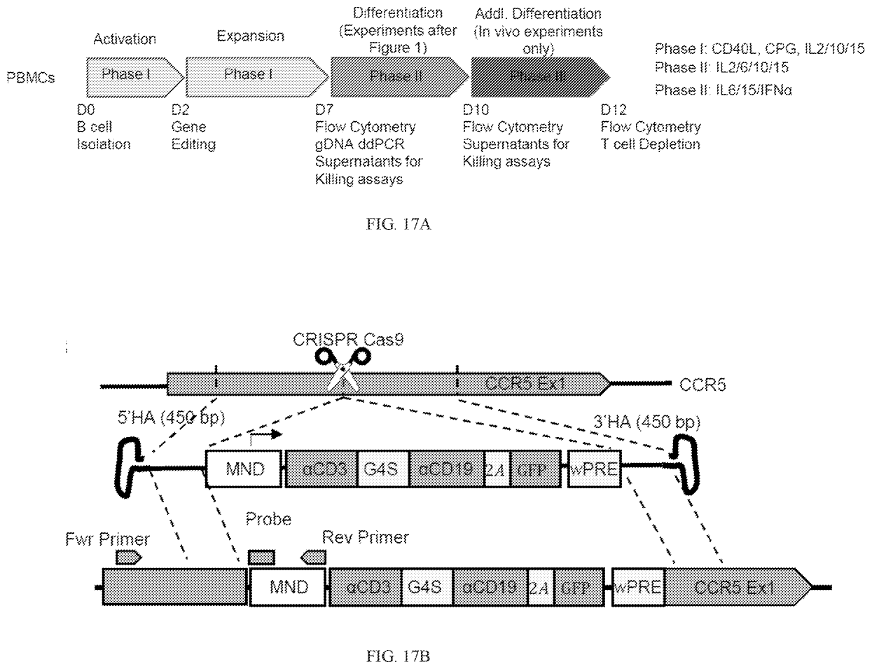

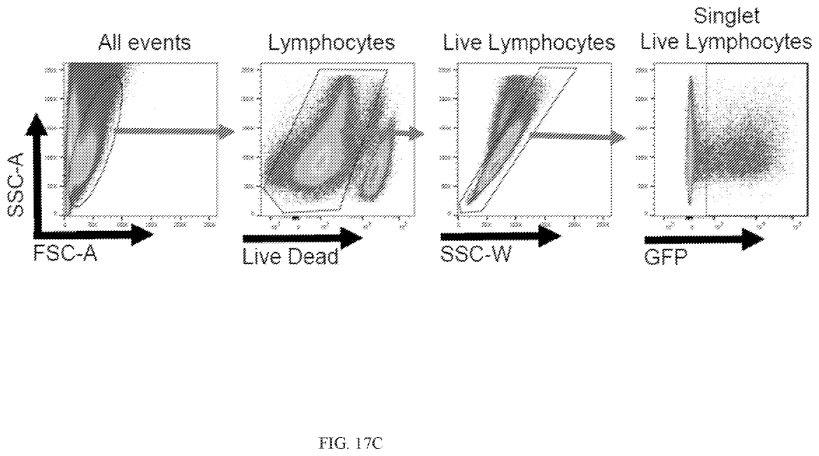

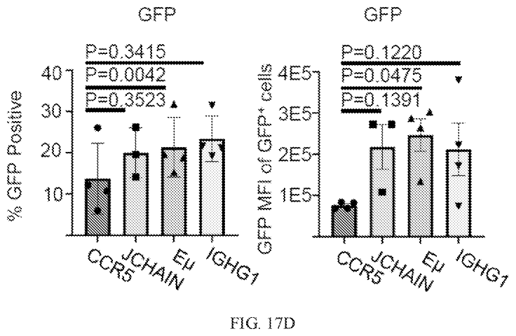

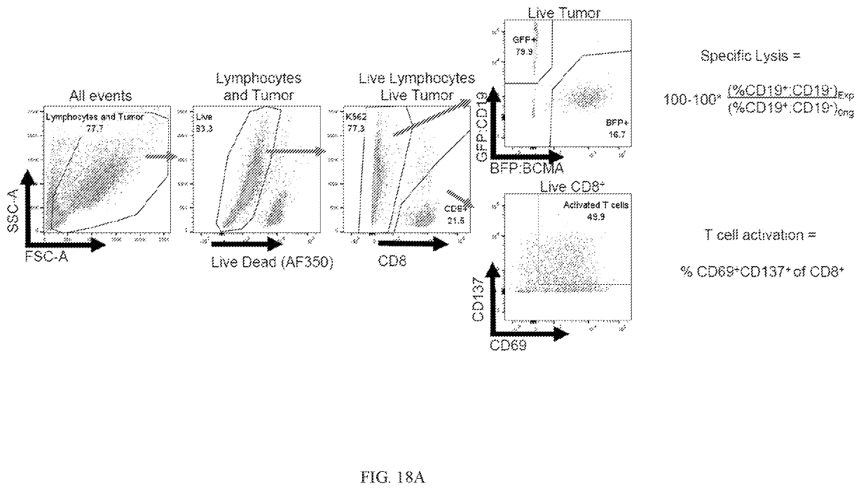

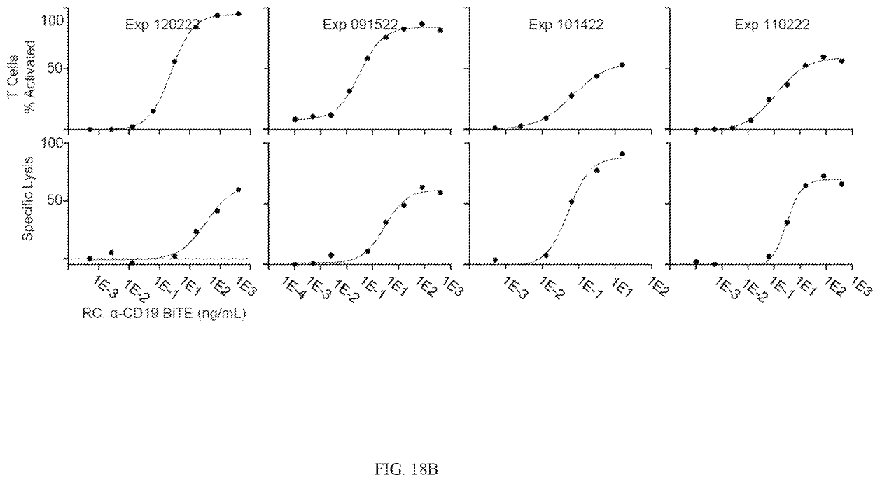

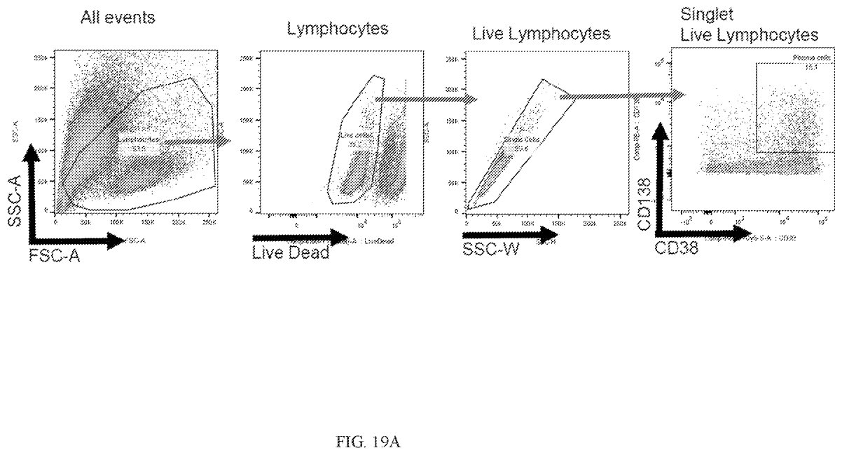

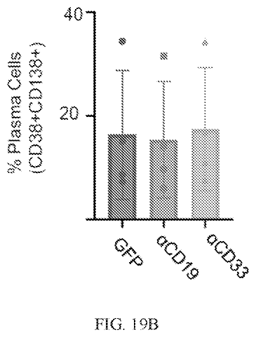

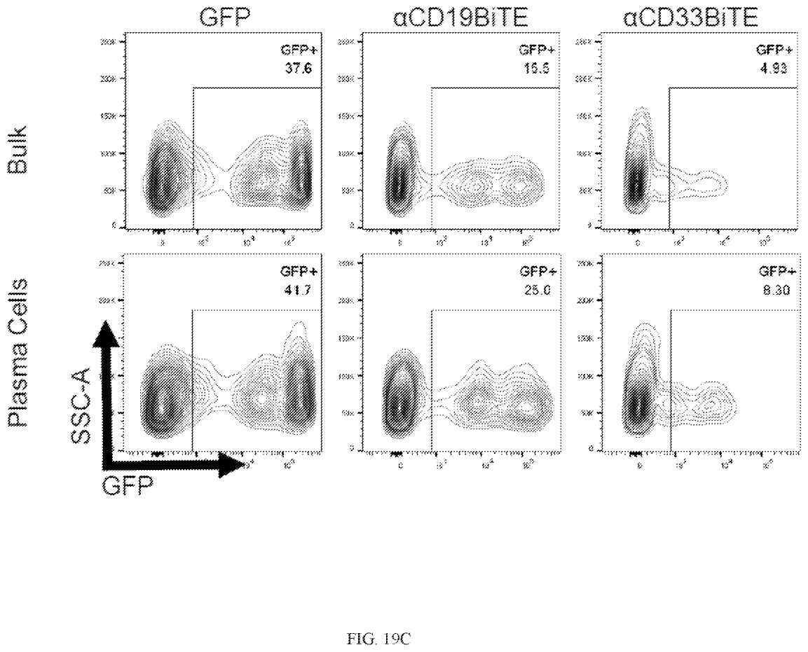

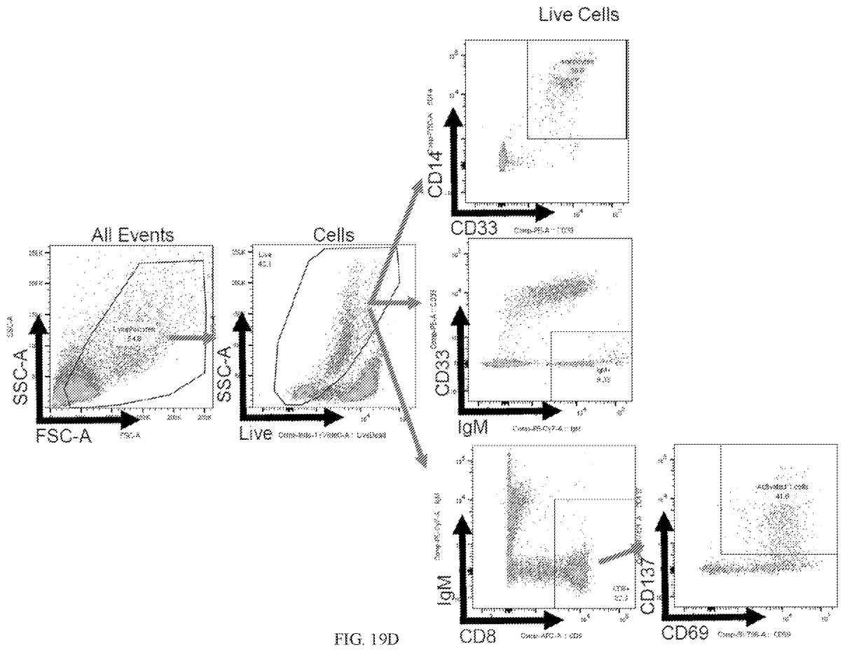

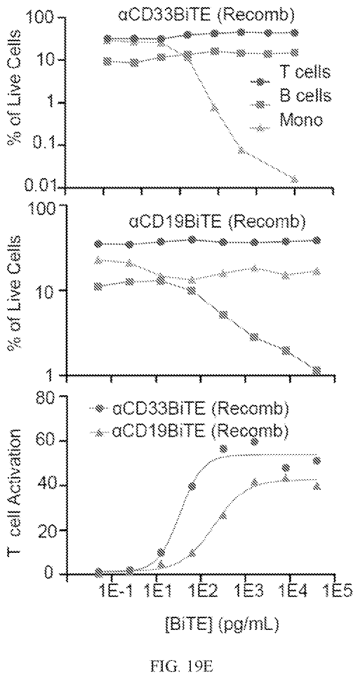

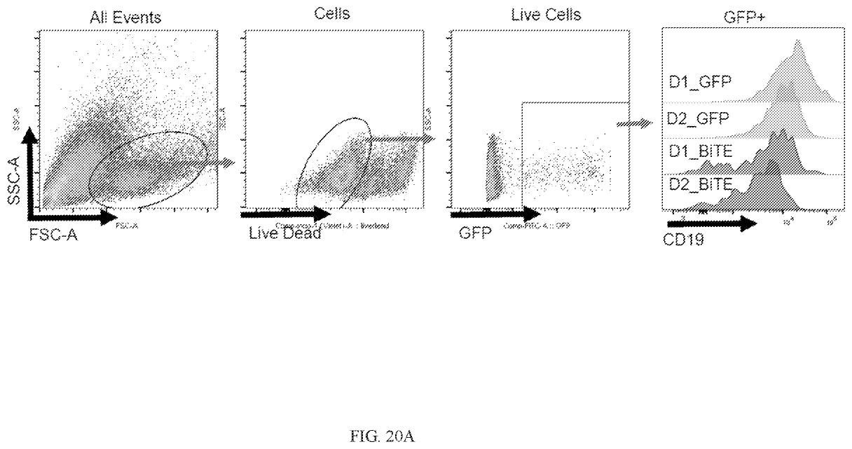

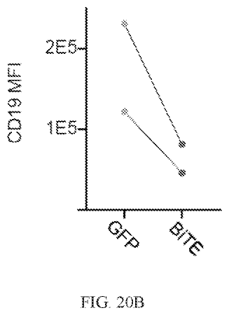

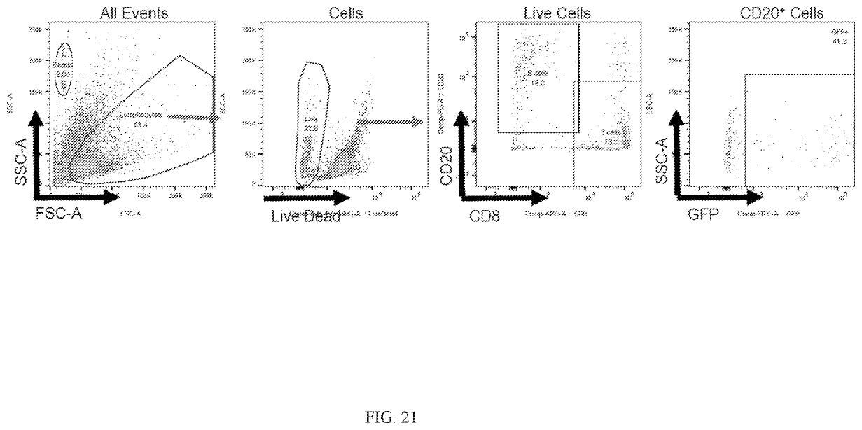

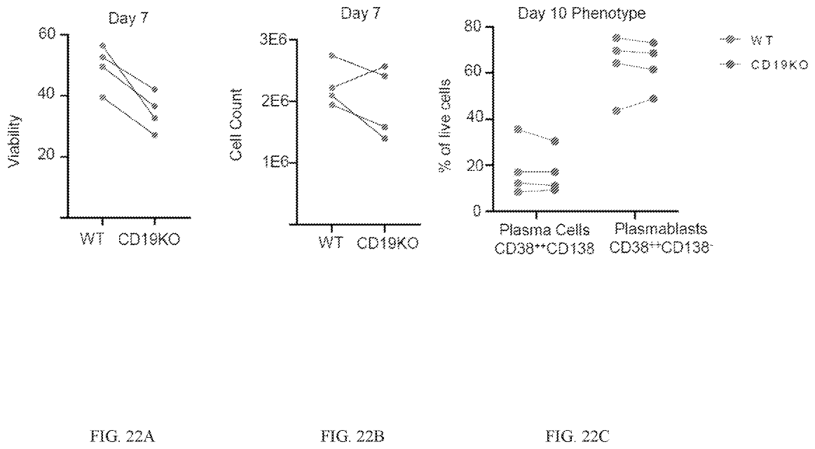

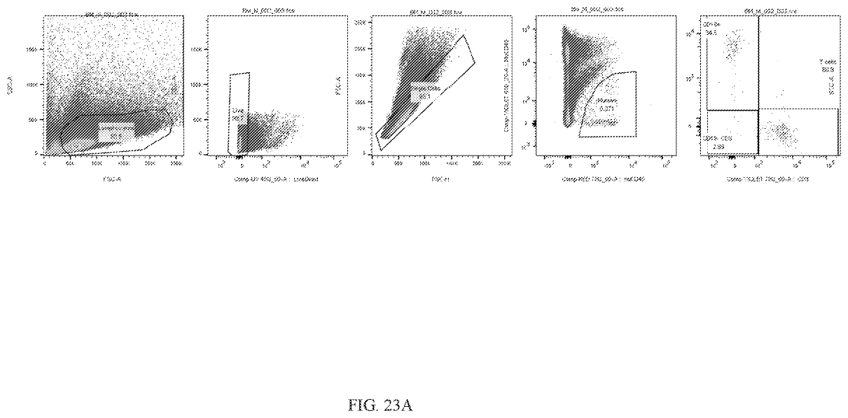

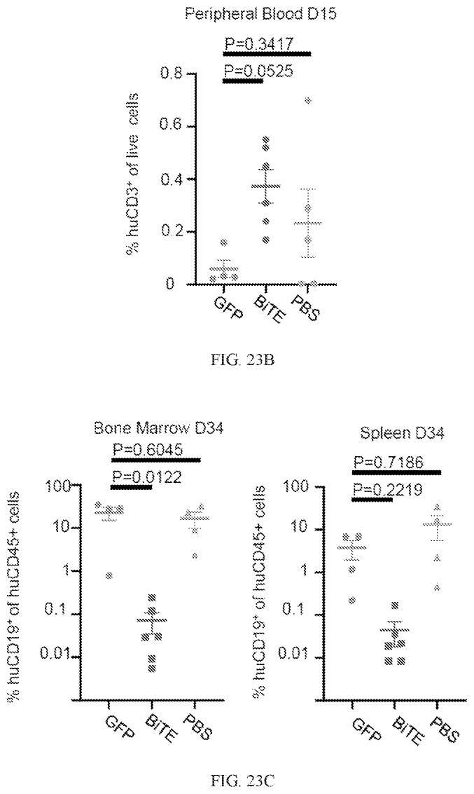

˜9-Five days later genomic DNA, cells and supernatants were harvested. FIG. 13B depicts a graph of transgene integration at CCR5 locus as HDR allele frequency measured by ddPCR. FIG. 13C depicts a flow cytometry analysis which was performed to generate representative flow plots of engineered B cells and to quantify % transgene+ of live cells. FIG. 13D depicts a ratio of engineering rate calculated by ddPCR vs flow cytometry. FIG. 13E depicts a schematic in which B cells were edited with GFP or BiTE.T2A. GFP at additional antibody associated loci. FIG. 13F depicts representative flow analysis of BITE.T2A.GFP edited B cells. FIG. 13G depicts a graph of quantification of % edited and GFP mean fluorescent intensity. FIG. 13H depicts a schematic in which supernatants from edited B cells were incubated with target (CD19+) and reference (CD19−) K562 cells with CD8+ T cells for 48 hrs. Cells were harvested for flow cytometry. FIG. 13I depicts a graph for specific lysis of CD19+ K562. FIG. 13I depicts a graph in which T cell activation (% CD69+,CD137+ of CD3+ cells) was quantified.

FIG. 13K depicts a graph in which the concentration of BiTE in supernatants was back calculated from % T cell activated. Data across >3 unique biological donors in 5 independent experiments. Error bars represent SEM. (FIGS. 13D, 13F,13H-13J) One-way paired ANOVA with Dunnett's post-test.

FIGS. 14A-14J depict human plasma cells engineered to secrete various BiTES had specific anti-target efficacy. FIG. 14A depicts a schematic in which primary human B cells were enriched from PBMCs and cultured in activating expansion media for two days and then edited express GFP or αCD19BITE.T2A.GFP or αCD33BITE.T2A.GFP transgenes. Edited cells were cultured in expansionary media for 5 days followed by differentiation into plasma cells over 3 days. Integration and transgene expression was verified by flow cytometry. FIG. 14B depicts representative flow plots of the GFP fluorescent reporter. FIG. 14 depicts a graph in which the GFP fluorescent reporter was quantified. FIG. 14D depicts a schematic in which PBMCs and autologous CD8+ T cells were cultured in the presence of supernatants from edited plasma cells for 48 hours. FIG. 14E depicts T cell activation (% CD69+,CD137+ of CD3+ cells) quantified by flow cytometry. FIG. 14F depicts % B cells (IgM+) of live cells quantified by flow cytometry. FIG. 14G depicts % monocytes (CD14+) of live cells in PBMC cultures at the end of the 48-hour co-culture quantified by flow cytometry. FIG. 14H depicts T cell activation (% CD69+, CD137+ of CD8+ cells). FIG. 14I depicts frequency of NAML-6 (CD19+) in the leukemia killing assay. FIG. 14J depicts frequency of MOLM-14 (CD33+) in the leukemia killing assay. Data across >4 unique biological donors in 3 independent experiments, one-way paired ANOVA with Dunnett's post-test.

FIGS. 15A-15I depict CD19 knockout prevented self-targeting of αCD19-BITE-cells and increased αCD19-BiTE expression for primary human B cells, which were enriched from PBMCs and cultured in activating media for two days and then engineered cells expressed GFP or αCD19BITE.T2A.GFP at the MuE locus. FIG. 15A depicts a schematic in which edited cells were expanded for an additional 5 days and then incubated with autologous T cells. FIG. 15B depicts a graph in which after 24 hrs the percentage of GFP+CD20+B cells was determined by flow cytometry. An additional CD19-specific CRISPR guide was added during engineering to knock out CD19 while engineering into the MuE locus. FIG. 15C depicts a flow analysis in which successful CD19 knock out was determined staining for surface CD19 expression. FIG. 15D is a graph in which percentage CD19+ cells from FIG. 15C were quantified. FIG. 15E depicts a graph in which CD19KO cells were incubated with various amounts T cells for 24 hours, and lack of αCD19BITE self-targeting in CD19KO cells was determined by flow cytometry by quantifying the percent of cells that are transgene positive.

FIG. 15F depicts a graph for combined data at a T to B cell ratio of 9 in which edited B cells were further differentiated over 3 days into plasma cells; supernatants from CD19KO and WT αCD19BITE edited plasma cells were incubated with T cells, K562 CD19+ and K562 CD19-cells for 48 hours. FIG. 15G depicts a graph in which specific lysis of CD19+ K562 was quantified FIG. 15H depicts a graph in which T cell activation (% CD69+,CD137+ of CD3+ cells) was quantified. FIG. 15I depicts a graph in which αCD19BiTE concentration was interpolated using recombinant &CD19BITE standards curve. Data across 4 unique biological donors. Matched two-way ANOVA (B,E). Matched one-way ANOVA with Dunnett's post-test (FIG. 15F). Paired student's T test (FIGS. 15G-15I).

FIGS. 16A-16E depicts plasma cells engineered to secrete BiTES had anti-tumor efficacy in vivo. FIG. 16A depicts a schematic in which either GFP or αCD19 BITE CD19KO engineered plasma cells, autologous T cells and Raji-ffLuciferase cells were injected subcutaneous into the right flank of NSG mice. FIG. 16B depicts Raji tumor growth was measured by bioluminescence via in vivo imaging studies until saturation occurred in control mice after day 9. FIG. 16C (left panel) depicts a graph for tumor bioluminescence for each mouse quantified over time; FIG. 16C (right panel) depicts an area under curve analysis with baseline correction 660,000 flux. FIG. 16D depicts a schematic in which either GFP or αCD19 BITE CD19KO engineered plasma cells were injected intravenous into NSG mice followed by intravenous injection with human B-ALL PDX NL482b-ffLuciferase cells followed by retroorbital injections of autologous T cells according to schema. FIG. 16E depicts disseminated PDX B-ALL growth which was monitored by in vivo imaging studies at until saturation occurred in control mice after day 17. FIG. 16F depicts a graph in which tumor growth was quantified via total bioluminescent flux over time for each mouse. FIG. 16G depicts a graph in which area under the curve analysis was conducted with baseline correction 1,000,000. FIGS. 16A-16C, data across 4 unique donors in two independent experiments (n=16) tested by paired two-way ANOVA. FIGS. 16D-16G, data from one biologic donor (n=15) tested by one-way unpaired ANOVA with Šídák's post-test.

FIGS. 17A-17D depict additional data related to the generation of genome engineered B cells and subsequent differentiation to plasma cells. FIG. 17A depicts a schematic in which B cells were enriched from human PBMCs and cultured in activating media for two days. B cells were then engineered using homologous directed repair. Engineered cells were further expanded for 5 days. FIG. 13 experiments only: engineered expanded B cells were harvested for ddPCR, flowcytometry, and supernatants for killing assays. Cells were then cultured at 1E6/mL in cytokines driving differentiation towards plasma cells. FIG. 17B depicts a schematic in which homology directed repair resulted in transgene directly integrated at the CCR5 gene. Integrated alleles and reference alleles were measured by ddPCR and used to calculate HDR frequency in FIG. 13B. FIG. 17C depicts a flow analysis with a gating strategy for determining the % GFP and GFP MFI of edited B cells at Day 7. Flow data was first gated on lymphocytes by SSCA vs FSCA. Lymphocytes were then gated on live cells. FIG. 17D depicts graph in which live lymphocytes were then gated for single cells and analyzed for GFP expression; percent % GFP+ (left panel) was calculated from singlet live cells, and the mean fluorescent index (right panel) was calculated from GFP+ cells.

FIGS. 18A-18B depict recombinant BiTE Dose dependent T cell activation and K562 killing in which various concentrations of recombinant «CD19BiTE were cultured with target (GFP+: CD19+) and reference (BFP+: CD19-) K562 cells and CD8+ T cells for 48 hrs. Cells were harvested for analysis by flow cytometry. FIG. 18A depicts a gating strategy for flow cytometry of K562 killing assay after 48 hours. T cell activation was defined as the percent of live CD8+ cells that are CD69+CD137+. Specific lysis was calculated using the ratio of GFP+:CD19+ to BFP+:CD19− cells normalized to the same ration in the media only control. FIG. 18B depicts graphs in which standards curves were interpolated for each experiment. Standards curves were then used to back calculate the concentration of secreted αCD19BITE in FIG. 13J.

FIGS. 19A-19E depict cells differentiated to day 10 maintain transgene expression and expressed phenotypic markers of plasma cells. Recombinant CD19 BiTE αCD33 BITE eliminate target cells in a dose dependent manner B cells were enriched from human PBMCs and cultured in activating media for two days. B cells were then engineered using homologous directed repair to express GFP or αCD19BITE.T2A.GFP or αCD33BiTE.T2A.GFP transgenes. Engineered cells were further expanded for 5 days and then cultured at 1E6/mL in cytokines driving differentiation towards plasma cells into plasma cells for an additional 3 days. Engineered plasma cells were harvested flowcytometry, and supernatants for killing assays. FIG. 19A depicts a gating strategy for flow cytometry analysis of cell phenotype. FIG. 19B depicts a graph for quantification of plasma cell phenotype (CD38+CD138+) of singlet live lymphocytes. FIG. 19C depicts flow analysis for representative and quantification of transgene positivity in bulk D10 cells or in the CD38+CD138+ plasma cells subset. PBMCs and autologous CD8+ T cells were cultured in the presence of either recombinant αCD19 BiTE or recombinant αCD33 BiTE for 48 hours. FIG. 19D depicts a flow analysis in which cells were harvested and analyzed using the gating strategy. FIG. 19E depicts graphs in which cells frequencies were quantified and plotted by concentration of either αCD19 BiTE or αCD33 BiTE. Standards curves were interpolated by four parameter logistic based on T cell activation data.

FIGS. 20A-20B depicts CD19 surface staining was lower αCD19 BITE engineered plasma cells. Primary human B cells were enriched from PBMCs and cultured in activating media for two days and then engineered express GFP or αCD19BiTE. T2A.GFP at the MuE locus. Engineered cells were then expanded and subsequently differentiated into plasma cells over an additional 8 days. FIG. 20A depicts a gating strategy to analyzed CD19 expression on engineered day 10 cells. FIG. 20B depicts a graph for quantification of CD19 mean fluorescent on engineered cells.

FIG. 21 depicts a gating strategy for B cell self killing assay. Primary human B cells were enriched from PBMCs and cultured in activating media for two days and then engineered express GFP or αCD19BITE.T2A.GFP at the MuE locus with or without CD19 knock out. Edited cells were expanded for an additional 5 days and then incubated with autologous T cells. After 24 hrs the percentage of edited B cells was determined by flow cytometry according to the gating strategy.

FIGS. 22A-22C depict CD19KO did not affect differentiation of BiTE engineered B cells into plasma cells. Primary human B cells were enriched from PBMCs and cultured in activating media for two days and then engineered express GFP or αCD19BiTE. T2A.GFP at the MuE locus with or without CD19 knock out. Edited cells were expanded for an additional 5 days and subsequently differentiated into plasma cells. FIG. 22A depicts a graph for cell viability. FIG. 22B depicts a graph for cell count. FIG. 22C depicts a graph in which cells were stained for surface markers and quantified as plasma cells (CD38**CD138*) and plasmablasts (CD38*CD138-).

FIGS. 23A-23B depict BiTE Plasma cells drove T cell expansion and CD19+ eradication in vivo. Peripheral blood was collected 15 days, spleens and bone marrow collected 34 days post NL428b tumor inoculation. Red blood cells were lysed and remaining cells were stained for mouse and human surface markers. FIG. 23A depicts a gating strategy for determining cell percentages. FIG. 23B depicts a graph for the percent of CD3+ cells of singlet live cells. FIG. 23C depicts graphs for the percent CD19+ of live CD45+ singlet cells. One-way unpaired ANOVA.

### DETAILED DESCRIPTION

Some embodiments of the method and compositions provided herein relate to methods of preparing cells expressing bispecific T cell engagers (BTCEs), and the use of such cells in certain therapies. In some embodiments, the cells are B cells or B cell precursors. As used herein, ‘BTCE’ and ‘BiTE’ are used interchangeably and each refer to a bispecific T cell engager.

Immunotherapies that recruit cytotoxic T cells to kill cancer cells, such as BTCEs, have played a significant role in the improved survival rates for B-cell acute lymphoblastic leukemia (B-ALL) patients¹⁻³. Blinatumomab, which is marketed under the name Blincyto, is a CD3/CD19 BTCE that received FDA approval in 2014 for the treatment of relapsed refractory B-ALL^{4,5}. Blinatumomab is now used in multiple B-ALL settings, including frontline therapy, as a bridge to transplantation, consolidation therapy, and as a low toxicity alternative to chemotherapy regimens⁶. However, Blinotumamab and other BTCEs require long periods of continuous high dose infusion due to their short half-lives; the half-life of blinatumomab is 2.11 hours⁷. To maintain therapeutic levels and avoid dose related complications, blinatumomab is administered in four cycles of a continuous daily infusion for 28 days followed by 14 days off to treat minimal residual disease positive B-ALL⁵. However, this intensive regimen can be challenging for patients, especially those with limited hospital access^{8,9}.

Several methods are being developed to directly increase biologic half-life¹⁰, including conjugation with small molecules, fragment crystallizable domains, or albumin binding motifs. Half-life extending conjugates have been applied to BTCEs¹¹ however, it remains unclear whether these BTCE fusions will be effective or overcome the need for multiple continuous high-dose infusions. An alternative method for mitigating problems with biologic half-life is to deliver the protein using a long-lived engineered cell therapy. Use of engineered plasma cells (ePCs) has been explored, a putative cell therapy modality that has been leveraged in proof-of-concept studies to stably produce biologic drugs including anti-pathogen antibodies¹²⁻¹⁶, immune-checkpoint inhibitors¹⁷, cytokines¹⁸, and proteins related to protein deficiency disorders¹⁹. Plasma cells are uniquely suited to deliver biologics over long periods due to their incredible lifespan²⁰ (estimated to be 11 to 200 years²¹), and high secretory capacity (up to 10,000 IgG molecules a second^{22,23}), Furthermore, ex-vivo generated ePCs resemble endogenous plasma cells, and can stably secrete therapeutically relevant levels of IgG for greater than one year in hIL6-humanized mice¹⁸. Because plasma cells^{24,25} and ePCs¹⁸ preferentially localize to bone marrow and other tissue microenvironments where progenitor B-ALL cells are likely to reside²⁶, as disclosed herein, it is predicted that ePCs will also benefit from local BTCE delivery to tumor sites in B-ALL.

Some embodiments disclosed herein include a homology directed repair strategy for the generation of ePC that produces large quantities of BTCEs. Findings disclosed herein demonstrated that ePCs secreting BTCEs can promote T-cell driven killing of cell lines, primary cells and patient-derived B-ALL xenografts. Some embodiments include use of ePCs in people for stable delivery of Blinatumomab in B-ALL, and use of the ePC platform for biologic delivery in cases where half-life is limiting or local delivery could reduce on-target adverse effects.

BTCEs may be rapidly cleared in a subject, thus BTCEs may have short half-life in a subject. Traditionally, BTCEs have been administered to a subject in multiple doses over an extended period of time, and/or by continuous intravenous infusion to maintain its therapeutic concentration (See e.g., Zhu M, et al., (2016) Clin Pharmacokinet. 55:1271-88; and Singh K, et al., (2021) Journal for Immuno Therapy of Cancer 9: e003679. doi: 10.1136/jitc-2021-003679). Thus there is a need for improved therapies relating to BTCEs.

Bispecific antibodies target two different epitopes, often on two different antigens, and have been described as “a key component of the next generation of antibody therapy”. See, for example, Wang et al., Antibodies 8:43, 2019. Various formats of bispecific antibodies have been developed. Commercial development of bispecific antibodies has been described as having been “hampered” by “great production challenges”.

Some embodiments provided herein relate to an insight that certain challenges associated with production and/or delivery of bispecific antibodies, and/or other multi-specific binding agents, such as different formats, may be overcome through development of engraftable human cell populations, and in particular engraftable human plasma cell populations, that can express such.

A variety of formats for bispecific antibody agents have been developed including five different structural categories: (i) bispecific IgG (BsIgG) (ii) IgG appended with an additional antigen-binding moiety (iii) BsAb fragments (iv) bispecific fusion proteins and (v) BsAb conjugates. See e.g., Spiess et al. (2015) Mol. Immunol. 67:95-106. An example bispecific antibody format include BTCEs, in which an scFv targeting CD3 on T cells is linked to, such as expressed as a single polypeptide with, an scFv targeting an antigen of interest, such as a surface antigen present on tumor cells. See e.g., Mack et al. (1995) Proc. Natl. Acad. Sci USA 92:7021. Blinatumomab is an example BTCE antibody that has achieved impressive efficacy in the treatment of B cell malignancies. See e.g., Zhou et al., (2021) Biomarker Res 9:38. A need to provide improved approaches to BTCE therapy is as an urgent issue, especially for solid timor is which response to BTCE therapy in always poor. Particular challenges associated with achieving effective BTCE therapy include antigen loss and immunosuppressive factors such as the upregulation of immune checkpoints.

Some embodiments provided herein relate to improved strategies for delivering multispecific binding agent therapy, specifically including BTCE therapy, through administration of engineered cell populations, such as engineered mammalian cell populations, and in particular engineered human cell populations, which express a multispecific binding agent, such as a BTCE, agent of interest to a subject in need thereof, such as a subject having a cancer, such as having a tumor expressing/having been determined to express an antigen targeted by the agent—and/or otherwise expressing such antigen on cells and/or tissues that would benefit from T-cell targeting.

As described herein, engineered plasma cell populations can effectively deliver a multispecific binding agent, such as a BTCE agent to a subject, such as a mammal, such as a human.

Some embodiments provided herein include aspects for providing engineered plasma cell populations such as those described in US20180282692 which is incorporated by reference in its entirety.

In some embodiments, mammalian, such as human, cell populations may be engineered to express a multispecific binding agent, such as a BTCE agent) using technologies as described, for example, in U.S. 2016/0289637, US20190352614, U.S. 20210198344, WO 2022/006309, which are each expressly incorporated by reference in its entirety.

In some embodiments, constructs encoding and/or engineered cell populations expressing, or capable of expressing, once administered to and/or engrafted in, a recipient mammal, a multi-specific binding agent, such as a BTCE agent, may be characterized for example, in that when assessed in an engrafted mouse model as described herein, achieve expression, such as long-term expression, of the multi-specific binding agent in the mammal. Moreover, in some embodiments, such constructs and/or cell populations are characterized in that, when assessed in an engrafted mouse model as described herein, achieve reduction in tumor size and/or in one or more markers of ill health, in the mammal.

Some embodiments of the methods and compositions provided herein include an engraftable engineered mammalian plasma cell population expressing a BTCE agent. In some embodiments, the cell population is characterized in that, when administered to a mammal, achieves expression of the BTCE agent in the mammal. In some embodiments, the cell population is characterized in that, when administered to a mammal having a tumor expressing an antigen target of the BTCE agent, achieves successful inhibition or treatment of the tumor and/or improvement of the mammal's health relative absence of such administration. Some embodiments of the methods and compositions provided herein include a method of inhibiting or treating a tumor expressing an antigen by administering to a mammal having such tumor any one of the foregoing cell populations. Some embodiments of the methods and compositions provided herein include construct encoding a BTCE agent for expression of the agent from engineered mammalian cells into which the construct is introduced.

### Definitions

As used herein, “B cells” or “B lymphocytes” have their plain and ordinary meaning when read in light of the specification, and may include but is not limited to, for example, a white blood cell type of the lymphocyte subtype. B cells are unlike lymphocytes such as T cells and natural killer cells, as B cells express B cell receptors on their cell membrane. The B cell receptors allow the B cell to bind a specific antigen, which will initiate an antibody response. B cells develop from hematopoietic stem cells. As described herein, B cells can include B cell precursors, stem cells, early pro-B cells, late pro-B cells, large pre-B cells, small pre-B cells, immature B cells, T1 B cells, T2 B cells, marginal zone B cells, mature B cells, naïve B cells, activated B cells derived from any starting B cell population, plasmablasts (short-lived) cells, GC B cells, memory B cells, and/or long or short-lived plasma cells and/or any mixtures or combinations thereof depending on the context.

As used herein, B cell precursors include the cells from which the B cells are derived. Like T cells, B cells are lymphatic cells that are originated from the bone marrow, where they can reside until they are mature. The B cells, as described in the alternatives herein, include stem cells, early pro-B cells, late pro-B cells, large pre-B cells, small pre-B cells, immature B cells, T1 B cells, T2 B cells, marginal zone B cells, mature B cells, naïve B cells, plasmablasts (short-lived) cells, GC B cells, memory B cells, plasmablast cells and/or long lived plasma cells. In some alternatives of the plasma cell for expressing a molecule such as a macromolecule, protein or a peptide, the plasma cell is derived from a B cell. In some alternatives, the B cell is a memory B cell. In some alternatives, the B cell is a stem cell, early pro-B cell, late pro-B cell, large pre-B cell, small pre-B cell, immature B cell, T1 B cell, T2 B cell, marginal zone B cell, mature B cell, naïve B cell, plasmablasts (short-lived) cell, GC B cell, memory B cell, plasmablast cell or a long lived plasma cell. In some alternatives, the B cells comprise B cell precursors such as hematopoietic stem cells (HSCs), multipotent progenitor (MPP) cells, lymphoid progenitor (CLP) cells, naïve B cells, GC B cell, plasmablasts, early pro-B cells, late pro-B cells, large pre-B cells, small pre-B cells, immature B cells, T1 B cells, T2 B cells, marginal zone B cells, mature B cells and/or memory B cells. In some alternatives, the macromolecule is a prodrug.

As used herein, “Memory B cells” have their plain and ordinary meaning when read in light of the specification, and may include but is not limited to, for example, the B cell sub-types that are formed within germinal centers following primary infection and are important in generating an accelerated and more robust antibody-mediated immune response in the case of re-infection. The B lymphocytes form the memory cells that can remember the same pathogen for future antibody production during future infections. In some alternatives of the plasma cell for expressing a molecule such as a macromolecule, protein or a peptide, the plasma cell is derived from a B cell. In some alternatives, the B cell is a memory B cell. In some alternatives, the macromolecule is a prodrug.

As used herein, “Naïve B cell” has its plain and ordinary meaning when read in light of the specification, and may include but is not limited to, for example, a B cell that has not yet been exposed to an antigen. Once exposed to an antigen, the naïve B cell becomes a memory B cell. In some alternatives of the plasma cell for expressing a molecule such as a macromolecule, protein or a peptide, the plasma cell is derived from a B cell. In some alternatives, the B cell is a memory B cell. In some alternatives, the macromolecule is a prodrug. “Peripheral blood mononuclear cells” (PBNC) as described herein are peripheral blood cells having a round nucleus. These cells consist of lymphocytes (T cells, B cells, NK cells) and monocytes, whereas erythrocytes and platelets have no nuclei, and neutrophils, basophils, and eosinophils have multi-lobed nuclei. In the alternatives described herein, the B cells are subject derived or are allogeneic peripheral blood mononuclear cells. In some alternatives, the B cells are blood-derived human B cells.

As used herein, “Plasma cells” as described herein, are also called plasma B cells, plasmocytes, plasmacytes, or effector B cells. Plasma cells have their plain and ordinary meaning when read in light of the specification, and may include but is not limited to, for example, white blood cells that secrete antibodies and are transported by the blood plasma and the lymphatic system.

As used herein, “Plasma cell precursor” can begin as an immature plasma cell. The most immature blood cell of the plasma cell lineage is called the plasmablast which can differentiate into a mature fully differentiated plasma cells. Plasmablasts can secrete more antibodies than a B cell, but less than a plasma cell. In some alternatives, a method of making a plasma cell that expresses a molecule is provided. In some alternatives, the plasma cell is a plasma cell precursor. In some alternatives, the plasma cell precursor is a plasmablast.

“Genome editing” has its plain and ordinary meaning when read in light of the specification, and may include but is not limited to, for example, a process that include methods for genetic engineering in which DNA is inserted, deleted or replaced in the genome of a living organism. Editing a gene is also known as gene editing. In some alternatives described herein, a method of making plasma cells or plasma cell precursors that express a molecule, such as a macromolecule is provided, in which B cells or B cell precursors are subjected to at least one round of genome editing. Methods of genome editing can include, but is not limited to nucleic acid being inserted, deleted or replaced in the genome of a cell. In some alternatives, a nuclease is used to achieve this process. In some alternatives, the nuclease is engineered. In some alternatives, the methods include inducing double strand breaks that are repaired through nonhomologous end-joining (NHEJ) or homologous recombination (HR). In some alternatives, the step of genome editing is performed by introduction of a single stranded nucleic acid. In some alternatives, the at least one round of genome editing further comprises cycling the B-cells for homologous recombination of the single stranded DNA oligonucleotides or recombinant adeno-associated virus into the candidate genetic loci. In some alternatives, the genome editing of the B cells for protein expression is performed in the absence of viral integration. In some alternatives, a second round of genome editing is performed to excise a region. In some alternatives, a third round of genome editing is performed to result in expression of a drug activatable growth enhancer. In some alternatives herein, the genome editing is performed by nonpathogenic AAV mediated editing by direct homologous recombination.

Genome editing can also employ the use of RNA and protein based transfection. For example the CRISPR/Cas system can be modified to edit genomes. This technique requires the delivery of the Cas nuclease complexed with a synthetic guide RNA (gRNA) into a cell, thus the cell's genome can be cut at a specific location and allow existing genes to be removed and/or add new ones. Thus, CRISPR/Cas and related programmable endonuclease systems are rapidly becoming significant genome editing tools of the biomedical research laboratory, with their application for gene disruption and/or gene targeting as demonstrated in a variety of cultured cell and model organism systems. In some alternatives, of the CRISPR/Cas system described herein, the Cas nuclease comprises Cas1, Cas2, Cas3, Cas4, Cas5, Cas6, Cas7, Cas8 or Cas9.

The basic components of CRISPR/Cas system comprise a target gene, a protospacer adjacent motif (PAM), a guide RNA, Cas endonuclease. An important aspect of applying CRISPR/Cas for genome editing is the need for a system to deliver the guide RNAs efficiently to a wide variety of cell types. This could, for example, involve delivery of an in vitro generated guide RNA as a nucleic acid (the guide RNA generated by in vitro transcription or chemical synthesis). In some alternatives, the nucleic acid could be rendered nuclease resistant by incorporation of modified bases.

The CRISPR-Cas system falls into two classes. The Class 1 system has a complex of multiple Cas proteins for the degradation of foreign nucleic acids. The Class 2 system has a single large Cas protein for a same purpose for the degradation of foreign nucleic acids. There are a 93 cas genes that are grouped into 35 families. 11 of the 35 families from a cas core which includes the protein families CAS1 to CAS9. As described herein, Cas comprises Cas1, Cas2, Cas3, Cas4, Cas5, Cas6, Cas7, Cas8 or Cas9.

Gene editing may also be performed by a novel non-nuclease based gene editing platform. A novel family of AAVs were previously isolated from human hematopoietic stem cells. These nonpathogenic AAVs are naturally present in healthy individuals and may possess unique gene editing and gene transfer properties. This technique is also described as AAV mediated editing by direct homologous recombination (AmENDR™). This process is homologous recombination by a natural biological mechanism that is used by cells to ensure highly precise DNA repair.

AAV mediated editing by direct homologous recombination is initiated by design of homology sequence “arms” that are specific to a region of the genome and results in a permanent correction in the DNA when administered to cells. In some alternatives herein, the gene editing is performed by nonpathogenic AAV mediated editing by direct homologous recombination. The identification of novel AAV genomes are described in Smith et al. (Mol Ther. 2014 September; 22 (9): 1625-1634; incorporated by reference in its entirety herein). The novel AAVs described by Smith et al., represents a new class of genetic vector for the manipulation of HSC genomes. Furthermore, these vectors may greatly expand the ability to deliver genes to targeted tissues and cells including cells that are refractory to gene transfer which circumventing prevalent preexisting immunity to AAV2. In some alternatives, the gene editing is performed by nonpathogenic AAVs naturally present in hematopoietic cells, wherein the editing is performed by AAV mediated editing by direct homologous recombination using the nonpathogenic AAVs as described in Smith et al.

“Engineered nucleases” have their plain and ordinary meaning when read in light of the specification, and may include but is not limited to, for example, enzymes that are engineered to be hybrid enzymes which can be used to specifically recognize a DNA sequence and efficiently edit the genome by the introduction of double-strand breaks. Without being limiting, there are four families of engineered nucleases are meganucleases, zinc finger nucleases (ZFN), transcription activator like effector-based nucleases (TALEN), and the CRISPR-Cas system.

“Meganucleases” have their plain and ordinary meaning when read in light of the specification, and may include but is not limited to, for example, endodeoxyribonucleases characterized by a large recognition site (double-stranded DNA sequences of 12 to 40 base pairs). In some alternative methods for making a plasma cell or plasma cell precursor that expresses a molecule such as a macromolecule, the method comprises: (a) isolating B cells, (b) developing the B cells, (c) performing a first round of genome editing of the B cells for protein expression in absence of viral integration, (d) expanding the B cells, and (e) differentiating the B cells, optionally, after step (c) or (d), thereby producing plasma cells that express a protein. In some alternatives, the first round of genome editing is performed by an RNA and protein based transfection. In some alternatives, the nuclease is a meganuclease.

“Zinc finger nucleases (ZFN)” have their plain and ordinary meaning when read in light of the specification, and may include but is not limited to, for example, engineered restriction enzymes that are generated by fusing a zinc finger DNA-binding domain to a DNA-cleavage domain. Zinc finger domains can be engineered to target specific desired DNA sequences and this enables zinc-finger nucleases to target unique sequences within complex genomes. In some alternative methods for making a plasma cell that expresses a molecule, such as a macromolecule, the method comprises: (a) isolating B cells, (b) developing the B cells, (c) performing a first round of genome editing of the B cells for protein expression in absence of viral integration, (d) expanding the B cells, and (e) differentiating the B cells, optionally, after step (c) or (d), thereby producing plasma cells that express a protein. In some alternatives, the first round of genome editing is performed by an RNA and protein based transfection. In some alternatives, the nuclease is a zinc finger nuclease.

“Transcription activator-like effector nucleases,” (TALEN), have their plain and ordinary meaning when read in light of the specification, and may include but is not limited to, for example, restriction enzymes that can be engineered to cut specific sequences or sites in DNA. They are made by fusing a TAL effector DNA-binding domain to a DNA cleavage domain (a nuclease which cuts DNA strands). Transcription activator-like effectors (TALEs) can be engineered to bind to a desired DNA sequence, so when combined with a nuclease, the DNA can be cut at specific locations. Thus, the restriction enzymes can be introduced into cells, for use in genome editing or for genome editing in situ, a technique known as genome editing with engineered nucleases. The use of TALEN is known to those of skill in the art. In some alternatives described herein, a method of making plasma cells or plasma cell precursors that express a molecule, such as a macromolecule is provided, in which B cells or B cell precursors are subjected to at least one round of genome editing. Methods of genome editing can include, but is not limited to nucleic acid being inserted, deleted or replaced in the genome of a cell. In some alternatives, a nuclease is used to achieve this process. In some alternatives, the nuclease is engineered. In some alternatives, the methods include inducing double strand breaks that are repaired through nonhomologous end-joining (NHEJ) or homologous recombination (HR). In some alternatives, the method includes a first round of genome editing or genome editing. In some alternatives, the first round of genome editing comprises delivering a nuclease, wherein the nuclease targets at least one genetic loci in the B cell. In some alternatives, the at least one genetic loci comprises JCHAIN, IGKC, IGMC, PON3, PRG2, FKBP11, SDC1, SLPI, DERL3, EDEM1, LY6C2, CRELD2, REXO2, PDIA4, PRDM1, CARD11, CCR5 or SDF2L1. In some alternatives, the nuclease is a zinc finger nuclease, transcription activator-like effector nuclease (TALEN), homing endonuclease (HEs), combined TALEN-HE protein (megaTALs) or synthetic guide RNAs targeting clustered regularly interspersed short palindromic repeat DNA (CRISPR) coupled to a Cas nuclease. In some alternatives, the Cas nuclease comprises Cas1, Cas2, Cas3, Cas4, Cas5, Cas6, Cas7, Cas8 or Cas9. In some alternatives, the first round of genome editing comprises transducing the B cell with a recombinant adeno-associated virus vector to serve as a donor template for homologous recombination into a candidate genetic loci. In some alternatives, the recombinant adeno-associated virus vector is single-stranded, double stranded or self-complementary.

“Differentiation” as described herein, refers to a cell changing from one cell type into another. Without being limiting, B cells can be differentiated based on their exposure to T cell-derived cytokines bound by B cell cytokine receptors. For example, CD40L can serve as a necessary stimulatory factor for B cell activation by binding the B cell surface receptor CD40, which can also affect differentiation. In the alternative methods described herein, the B cell is differentiated in a three step culture system comprising activation and proliferation step, a plasmablast differentiation step, and a plasma cell differentiation step. In some alternatives, the activation and proliferation step is performed in the presence of any combination of MCD40L (CD40 trimer), CpG, IL-2, IL-10 and/or IL-15. In some alternatives, the plasmablast differentiation step is performed in the presence any combination of IL-2, IL-6, IL-10 and/or IL-15. In some alternatives, the plasma cell differentiation step is performed in the presence any combination of IL-6, IL-15, APRIL and/or IFNα.

### Certain Methods

Some embodiments of the methods and compositions provided herein include methods for modifying a cell to express a bispecific T-cell engager (BTCE). Example methods useful with certain embodiments provided herein are disclosed in U.S. 2018/0282692 which is incorporated by reference herein in its entirety. In some embodiments the cell is a B cell or a B cell precursor. In some embodiments, the B cell or B cell precursor is a hematopoietic stem cell, a human embryonic stem cell, an induced pluripotent stem cell (iPSC), a naïve B cell, a memory B cell, a plasmablast, or a plasma cell. In some such embodiments, the cell is a primary B cell or primary B cell precursor.

In some embodiments a cell is genetically modified to express the BTCE. For example, a cell genome can be modified by insertion of an expression cassette encoding the BTCE. In some embodiments, the insertion can be at a random location in the genome. In some embodiments, the insertion is targeted into a selected location of the genome. Some embodiments include use of homology directed repair (HDR). Some such embodiments can include an endonuclease capable of inserting an expression cassette at a selected location in a genome. Examples of such endonucleases include zinc finger nucleases, transcription activator-like effector nucleases (TALEN), homing endonucleases (HEs), combined TALEN-HE proteins (megaTALs) and CRISPR/Cas systems. In some embodiments, the CRISPR/Cas systems includes a Cas nuclease, such as Cas1, Cas2, Cas3, Cas4, Cas5, Cas6, Cas7, Cas8 or Cas9, and a guide RNA (gRNA) in which the gRNA is adapted to target a certain locus in a genome. In some embodiments, the Cas nuclease is Cas9.

In some embodiments, the expression cassette comprises a nucleic acid encoding the BTCE. In some embodiments, the expression cassette also includes a promoter operably linked to a nucleic acid encoding the BTCE. Examples of promoters include an MND promoter, an EF-1α promoter, or an IgHG1 promoter. In some embodiments, the expression cassette also includes an enhancer element, and/or a polyadenylation sequence.

Some embodiments of the methods provided herein for modifying a cell to express a BTCE include (a) obtaining a nuclease or nucleic acid encoding the nuclease capable of inserting an expression cassette into a locus in a B cell genome; and a first polynucleotide encoding a homology direct repair (HDR) template comprising the expression cassette, wherein the expression cassette encodes the BTCE; and (b) introducing into the cell the nuclease or nucleic acid encoding the nuclease and the first polynucleotide to obtain a modified cell. In some embodiments, the cell is activated prior to step (b). In some such embodiments, the activating comprises can include contacting the cell with an oligomerized CD40 ligand (CD40L) such as an oligomerized CD40L comprising two linked CD40L trimers, CpG, and/or IL-21. Some such embodiments can include maintaining the cell in culture medium containing oligomerized CD40L for a first period of time. In some embodiments, the first period of time is a period within a range from 1 day to 30 days, 1 day to 25 days, 5 days to 20 days, or 7 days to 20 days. In some embodiments, step (b) comprises contacting the cell with a ribonucleoprotein (RNP) comprising the nuclease and the gRNA.

Some embodiments also include step (c) differentiating the cell. In some embodiments, step (c) comprises a plasmablast differentiation step, and/or a plasma cell differentiation step. In some such embodiments, the differentiating is performed in the presence of any combination of oligomerized CD40L, CpG oligodeoxynucleotide, IL-2, IL-10 or IL-15. In some such embodiments, the plasmablast differentiation step is performed in the presence any combination of IL-2, IL-6, IL-10 or IL-15. In some such embodiments, the plasma cell differentiation step is performed in the presence any combination of IL-6, IL-15, APRIL or IFNα. In some embodiments, step (c) comprise (i) contacting the edited cell with IL-2, IL-6, IL-10 and IL-15 for a second period of time. In some embodiments, the second period of time is a period within a range from 1 day to 30 days, 1 day to 20 days, 1 day to 15 days, 1 day to 10 days, 1 day to 5 days, or 1 day to 3 days. Some embodiments also include (ii) contacting the edited cell with IL-6, IL-15 and IFNα for a third period of time. In some embodiments, the third period of time is a period within a range from 1 day to 30 days, 1 day to 20 days, 1 day to 15 days, 1 day to 10 days, 1 day to 5 days, or 1 day to 3 days. In some embodiments, step (ii) is performed after step (i). Example methods useful with embodiments of the methods and compositions provided herein are disclosed in Jourdan M., et al. (2009) Blood 114:5173-5181 which is incorporated by reference herein in its entirety.

In some embodiments, the nuclease is a Cas nuclease, such as a Cas9 nuclease. Some embodiments also include a second polynucleotide encoding a guide RNA (gRNA). In some embodiments, the second polynucleotide comprises a DNA or RNA sequence corresponding to a nucleotide sequence of any one of SEQ ID NOs: 01-27.

In some embodiments, the locus comprises an endogenous gene expressed at a higher level in a B cell than in a non-B cell. In some embodiments, the locus comprises an endogenous gene inactive in a B cell. In some embodiments, the locus is selected from a CCR5 gene, a JCHAIN gene, the IGHM locus (also known as E-mu; hg38 genome; chr14: 105856225-105863200), a CD19 gene, or an IGHG1 gene.

In some embodiments, the BTCE comprises a first polypeptide capable of specifically binding to a T-cell antigen, and a second polypeptide capable of specifically binding to a tumor antigen. In some embodiments, the T-cell antigen comprises CD3. In some embodiments, the tumor antigen is selected from CD33, CD19, CD326 (EpCAM), neuron-glial antigen 2 (NG2), HER2, epidermal growth factor receptor (EGFR), CD66e, ephrin type-A receptor 2 (EphA2), CD21, FLT3, gp100, PDL1, or CD22. In some embodiments, the BTCE comprises AMG 330, blinatumomab, solitomab, or tebentafusp.

In some embodiments, the expression cassette further comprises a promoter operably linked to the nucleic acid encoding the BTCE. In some embodiments, the promoter is a constitutive promoter. In some embodiments, the promoter is an inducible promoter. In some embodiments, the promoter is an MND promoter, an IgVH promoter, an EF-1α promoter, or an IgHG1 promoter. In some embodiments, the expression cassette further comprises: (i) an enhancer, optionally wherein the enhancer comprises an Eμ enhancer, or an SLC3A2 enhancer; (ii) a polynucleotide encoding a signal sequence, optionally wherein the signal sequence is an IgHV signal sequence or an IgHG1 signal sequence; (iii) a polynucleotide comprising a 5′ UTR from an IGHV gene or an IGHG1 gene; (iv) a polynucleotide comprising a 3′ UTR from an IGHV gene or an IGHG1 gene; and/or (v) a ubiquitous chromatin-opening element (UCOE). In some embodiments, the first polynucleotide further comprises a nucleic acid homologous to the locus; and optionally, wherein the nucleic acid homologous to the locus has a length in a range from about 200 to about 1500 consecutive nucleotides.

In some embodiments, a vector comprises the first polynucleotide. In some embodiments, the vector comprises a viral vector. In some embodiments, the viral vector is an adeno associate viral (AAV) vector, or a lentiviral vector. In some embodiments, the AAV vector is an AAV6 vector.

In some embodiments, the cell is mammalian. In some embodiments, the cell is human. In some embodiments, the cell is ex vivo. In some embodiments, the cell is in vitro. In some embodiments, the cell is autologous to a subject. In some embodiments, the cell is allogeneic to a subject. In some embodiments, the cell lacks expression of an endogenous protein to which the BTCE is capable of specifically binding. For example, a cell can be modified such that it lacks a ligand capable of binding the BTCE. Some such embodiments can be useful to inhibit or prevent binding of the BTCE to the cell, thereby inhibiting killing of the cell.

### Certain Compositions and Systems

Some embodiments of the methods and compositions provided herein relate to systems for modifying a cell to express a BTCE. Some such embodiments include (a) a nuclease or nucleic acid encoding the nuclease capable of inserting an expression cassette into a locus in a cell genome; and (b) a first polynucleotide encoding a homology direct repair (HDR) template comprising the expression cassette, wherein the expression cassette encodes the BTCE.

Some embodiments also include the cell, wherein the cell is a B cell or a B cell precursor. In some embodiments, the cell is selected from a hematopoietic stem cell, a human embryonic stem cell, an induced pluripotent stem cell (iPSC), a naïve B cell, a memory B cell, a plasmablast, or a plasma cell.

In some embodiments, the nuclease is a Cas nuclease; and optionally, wherein the nuclease is a Cas9 nuclease. Some embodiments also include a second polynucleotide encoding a guide RNA (gRNA). In some embodiments, the second polynucleotide comprises a DNA or RNA sequence corresponding to a nucleotide sequence of any one of SEQ ID NOs: 01-27.

In some embodiments, the locus comprises an endogenous gene expressed at a higher level in a B cell than in a non-B cell. In some embodiments, the locus comprises an endogenous gene inactive in a B cell. In some embodiments, the locus is selected from a CCR5 gene, a JCHAIN gene, the IGHM locus (also known as E-mu; hg38 genome; chr14: 105856225-105863200), a CD19 gene, or an IGHG1 gene.

In some embodiments, the BTCE comprises a first polypeptide capable of specifically binding to a T-cell antigen, and a second polypeptide capable of specifically binding to a tumor antigen. In some embodiments, the T-cell antigen comprises CD3. In some embodiments, the tumor antigen is selected from CD33, CD19, CD326 (EpCAM), neuron-glial antigen 2 (NG2), HER2, epidermal growth factor receptor (EGFR), CD66e, ephrin type-A receptor 2 (EphA2), CD21, FLT3, gp100, PDL1, or CD22. In some embodiments, the BTCE comprises AMG 330, blinatumomab, solitomab, or tebentafusp.

In some embodiments, the expression cassette further comprises a promoter operably linked to the nucleic acid encoding the BTCE. In some embodiments, the promoter is a constitutive promoter. In some embodiments, the promoter is an inducible promoter. In some embodiments, the promoter is an MND promoter, an IgVH promoter, an EF-1α promoter, or an IgHG1 promoter. In some embodiments, the expression cassette further comprises: (i) an enhancer, optionally wherein the enhancer comprises an Eμ enhancer, or an SLC3A2 enhancer; (ii) a polynucleotide encoding a signal sequence, optionally wherein the signal sequence is an IgHV signal sequence or an IgHG1 signal sequence; (iii) a polynucleotide comprising a 5′ UTR from an IGHV gene or an IGHG1 gene; (iv) a polynucleotide comprising a 3′ UTR from an IGHV gene or an IGHG1 gene; and/or (v) a ubiquitous chromatin-opening element (UCOE).

In some embodiments, the first polynucleotide further comprises a nucleic acid homologous to the locus; and optionally, wherein the nucleic acid homologous to the locus has a length in a range from about 200 to about 1500 consecutive nucleotides.

In some embodiments, a vector comprises the first polynucleotide. In some embodiments, the vector comprises a viral vector. In some embodiments, the viral vector is an adeno associate viral (AAV) vector, or a lentiviral vector. In some embodiments, the AAV vector is an AAV6 vector.

In some embodiments, the cell is mammalian. In some embodiments, the cell is human. In some embodiments, the cell is ex vivo. In some embodiments, the cell is autologous to a subject. In some embodiments, the cell is allogeneic to a subject. In some embodiments, the cell lacks expression of an endogenous protein to which the BTCE is capable of specifically binding.

Some embodiments of the methods and compositions provided herein include a cell prepared by any one of the methods provided herein.

Some embodiments of the methods and compositions provided herein include cell, wherein the cell is genetically modified at a genomic locus to express a BTCE. In some embodiments, the cell is a B cell or a B cell precursor. In some embodiments, the cell is selected from a hematopoietic stem cell, a human embryonic stem cell, an induced pluripotent stem cell (iPSC), a naïve B cell, a memory B cell, a plasmablast, or a plasma cell.

In some embodiments, the locus comprises an endogenous gene expressed at a higher level in a B cell than in a non-B cell. In some embodiments, the locus comprises an endogenous gene inactive in a B cell. In some embodiments, the locus is selected from a CCR5 gene, a JCHAIN gene, the IGHM locus (also known as E-mu; hg38 genome; chr14: 105856225-105863200), a CD19 gene, or an IGHG1 gene.

In some embodiments, the BTCE comprises a first polypeptide capable of specifically binding to a T-cell antigen, and a second polypeptide capable of specifically binding to a tumor antigen. In some embodiments, the T-cell antigen comprises CD3. In some embodiments, the tumor antigen is selected from CD33, CD19, CD326 (EpCAM), neuron-glial antigen 2 (NG2), HER2, epidermal growth factor receptor (EGFR), CD66e, ephrin type-A receptor 2 (EphA2), CD21, FLT3, gp100, PDL1, or CD22. In some embodiments, the BTCE comprises AMG 330, blinatumomab, solitomab, or tebentafusp.

In some embodiments, the genomic locus is modified by insertion of an expression cassette comprising a first nucleic acid encoding the BTCE. In some embodiments, the first nucleic acid is operably linked to an endogenous promoter at the locus. In some embodiments, the expression cassette further comprises a promoter operably linked to the first nucleic acid. In some embodiments, the promoter is a constitutive promoter. In some embodiments, the promoter is an inducible promoter. In some embodiments, the promoter is an MND promoter, an IgVH promoter, an EF-1a promoter, or an IgHG1 promoter. In some embodiments, the expression cassette further comprises: (i) an enhancer, optionally wherein the enhancer comprises an Eμ enhancer, or an SLC3A2 enhancer; (ii) a polynucleotide encoding a signal sequence, optionally wherein the signal sequence is an IgHV signal sequence or an IgHG1 signal sequence; (iii) a polynucleotide comprising a 5′ UTR from an IGHV gene or an IGHG1 gene; (iv) a polynucleotide comprising a 3′ UTR from an IGHV gene or an IGHG1 gene; and/or (v) a ubiquitous chromatin-opening element (UCOE).

In some embodiments, the cell is mammalian. In some embodiments, the cell is human. In some embodiments, the cell is ex vivo. In some embodiments, the cell is autologous to a subject. In some embodiments, the cell is allogeneic to a subject.

Some embodiments include a pharmaceutical composition comprising any one of the cells provided, and a pharmaceutically acceptable excipient. In some embodiments, the pharmaceutical composition is adapted for administration as an adoptive cell transfer. In some embodiments, the pharmaceutical composition is adapted for administration by injection at a site of a cancer, such as into a solid tumor.

### Certain Therapies

Some embodiments of the methods and compositions provided herein include methods for treating, ameliorating or inhibiting a disorder in a subject. In some embodiments, the subject is mammalian. In some embodiments, the subject is human.

Some such embodiments include administering to the subject a cell expressing a BTCE. In some embodiments, the cell is a cell genetically modified to express the BTCE. In some embodiments, the cell is a B cell or a B cell precursor. In some embodiments, the B cell or B cell precursor is a hematopoietic stem cell, a human embryonic stem cell, an induced pluripotent stem cell (iPSC), a naïve B cell, a memory B cell, a plasmablast, or a plasma cell. In some such embodiments, the cell is a primary B cell or primary B cell precursor. In some embodiments, the cell is mammalian. In some embodiments, the cell is human. In some embodiments, the cell is autologous to the subject. In some embodiments, the cell is allogeneic to the subject. In some embodiments, the cell is prepared by any one of the methods provided herein. In some embodiments, the cell lacks expression of an endogenous protein to which the BTCE is capable of specifically binding.

In some embodiments, the BTCE comprises a first polypeptide capable of specifically binding to a T-cell antigen, and a second polypeptide capable of specifically binding to a tumor antigen. In some embodiments, the T-cell antigen comprises CD3. In some embodiments, the tumor antigen is selected from CD33, CD19, CD326 (EpCAM), neuron-glial antigen 2 (NG2), HER2, epidermal growth factor receptor (EGFR), CD66e, ephrin type-A receptor 2 (EphA2), CD21, FLT3, gp100, PDL1, or CD22. In some embodiments, the BTCE comprises AMG 330, blinatumomab, solitomab, or tebentafusp.

In some embodiments, the disorder comprises a cancer, or an inflammatory disorder. In some embodiments, the cancer is a solid tumor or a leukemia. In some embodiments, the cancer is acute lymphoblastic leukemia (ALL), acute myeloid leukemia (AML), eye cancer, breast cancer, stomach cancer, melanoma, colon cancer, colorectal cancer, head and neck cancer, gastric cancer, prostate cancer, ovarian cancer, lung cancer, or pancreatic cancer.

In some embodiments, the cancer comprises a tumor antigen. In some embodiments, the tumor antigen is CD33, CD19, CD326 (EpCAM), neuron-glial antigen 2 (NG2), HER2, epidermal growth factor receptor (EGFR), CD66e, ephrin type-A receptor 2 (EphA2), CD21, FLT3, gp100, PDL1, or CD22.

In some embodiments, the tumor antigen is CD33, and the BTCE is AMG 330. In some such embodiments, the cancer comprises a CD33+ tumor cell. In some such embodiments, the cancer is an acute myeloid leukemia (AML), such as a relapsed/refractory AML.

In some embodiments, the tumor antigen is CD19, and the BTCE is blinatumomab. In some such embodiments, the cancer comprises a CD19+ tumor cell. In some such embodiments, the cancer is an acute lymphoblastic leukemia (ALL), such as a relapsed/refractory ALL.

In some embodiments, the tumor antigen is CD326 (EpCAM), and the BTCE is solitomab. In some such embodiments, the cancer comprises a CD326+ tumor cell. In some such embodiments, the cancer is gastrointestinal cancer or a lung cancer.

In some embodiments, the tumor antigen is a gp100/HLA-A*02:01 complex, and the BTCE is tebentafusp. In some such embodiments, the cancer comprises a tumor cell positive for a gp100/HLA-A*02:01 complex. In some such embodiments, the cancer is an eye cancer, such as an uveal melanoma.

BTCEs may be rapidly cleared in a subject, thus BTCEs may have short half-life in a subject. Traditionally, BTCEs have been administered to a subject in multiple doses over an extended period of time, and/or by continuous intravenous infusion to maintain its therapeutic concentration (See e.g., Zhu M, et al., (2016) Clin Pharmacokinet. 55:1271-88; and Singh K, et al., (2021) Journal for ImmunoTherapy of Cancer 9: e003679. doi: 10.1136/jitc-2021-003679). In contrast to prior methods, therapeutic methods provided herein include administration of a reduced or limited number of doses of a cell expressing a BTCE to a subject. For example, a subject can be administered no more than one, two, three, four, or five doses of a cell expressing a BTCE. In some embodiments, a subject can be administered no more than a single dose of a cell expressing a BTCE. In some embodiments, a dose of a cell expressing a BTCE is administered in a short period of time, in contrast to a continuous intravenous infusion to maintain its therapeutic concentration of prior methods. For example, the dose can be administered in less than 1 hour, 30 minutes, 20 minutes, 10 minutes, or 5 minutes.

Example nucleotide sequences for sgRNAs for genes including Eu, IGHG1, CCR5, JCHAIN, and CD19, and useful with certain embodiments of the methods and compositions provided herein are listed in TABLE 1.

TABLE 1

| SEQ ID NO | Nucleic acid/feature | Nucleotide sequence |
|---|---|---|
| SEQ ID NO: 01 | Emu sgRNA | GTCTCAGGAGCGGTGTCTGT |
| SEQ ID NO: 02 | IGHG1_sgRNA_1; exon 2 | TGAGTTTTGTCACAAGATTT |
| | (RJ178)) | |
| SEQ ID NO: 03 | IGHG1_sgRNA_2; exon 2 | GTGAGTTTTGTCACAAGATT |
| | (RJ180) | |
| SEQ ID NO: 04 | IGHG1_sgRNA_3; exon 2 | CACACATGCCCACCGTGCCC |
| | (RJ176) | |
| SEQ ID NO: 05 | IGHG1_sgRNA_4; exon 3 | CACCACGCATGTGACCTCAG |
| | (RJ182) | |
| SEQ ID NO: 06 | IGHG1_sgRNA_5; exon 3 | CCACCACGCATGTGACCTCA |
| | (RJ183) | |
| SEQ ID NO: 07 | IGHG1_sgRNA_6; exon 3 | AGGACACCCTCATGATCTCC |
| | (RJ185) | |
| SEQ ID NO: 08 | IGHG1_sgRNA_7; exon 3 | GGAAGACTGACGGTCCCCCC |
| | (RJ186) | |
| SEQ ID NO: 09 | IGHG1_sgRNA_8; exon 3 | ATGGTTTTCTCGATGGGGGC |
| | (RJ200) | |
| SEQ ID NO: 10 | IGHG1_sgRNA_9; exon 3 | GAGCAGTACAACAGCACGTA |
| | (RJ201) | |
| SEQ ID NO: 11 | IGHG1_sgRNA_10; exon 1 | GCTCTTGGAGGAGGGTGCCA |
| | (RJ173) | |
| SEQ ID NO: 12 | IGHG1_sgRNA_11; exon 1 | GTGCCAGGGGGAAGACCGAT |
| | (RJ174) | |
| SEQ ID NO: 13 | IGHG1_sgRNA_12; exon 4 | GTGCAGAGCCTCATGCATCA |
| | (RJ191) | |
| SEQ ID NO: 14 | IGHG1_sgRNA_13; exon 4 | CTCATGCTCCGTGATGCATG |
| | (RJ192) | |
| SEQ ID NO: 15 | IGHG1_sgRNA_14; exon 4 | TCACCGTGGACAAGAGCAGG |
| | (RJ193) | |
| SEQ ID NO: 16 | IGHG1_sgRNA_15; exon 4 | TTTGGAGATGGTTTTCTCGA |
| | (RJ194) | |
| SEQ ID NO: 17 | IGHG1_sgRNA_16; exon 4 | AGCCTGACCTGCCTGGTCAA |
| | (RJ195) | |
| SEQ ID NO: 18 | IGHG1_sgRNA_17; exon 4 | GGTCAGGCTGACCTGGTTCT |
| | (RJ196) | |
| SEQ ID NO: 19 | IGHG1_sgRNA_18; exon 4 | GTACACCCTGCCCCCATCCC |
| | (RJ197) | |
| SEQ ID NO: 20 | IGHG1_sgRNA_19; exon 4 | CCAGGCAGGTCAGGCTGACC |
| | (RJ198) | |
| SEQ ID NO: 21 | IGHG1_sgRNA_20; exon 4 | GGGGGCAGGGTGTACACCTG |
| | (RJ199) | |
| SEQ ID NO: 22 | CCR5_sgRNA; (RJ69) | CAATGTGTCAACTCTTGACA |
| SEQ ID NO: 23 | JCHAIN_sgRNA; (RJ94) | AAGAACCATTTGCTTTTCTG |
| SEQ ID NO: 24 | CD19_sgRNA_1; (RJ255) | CCTCGGGCCTGACTTCCATG |
| SEQ ID NO: 25 | CD19_sgRNA_2; RJ253 | CCTCGGGCCTGACTTCCATG |
| SEQ ID NO: 26 | CD19_sgRNA_3; RJ254 | GGAACCTCTAGTGGTGAAGG |
| SEQ ID NO: 27 | CD19_sgRNA_4; RJ256 | TTCACCACTAGAGGTTCCTC |
| SEQ ID NO: 28 | IGHG1_sgRNA_10; exon 1 | TTGTCCACCTTGGTGTTGCT |
| | (sgYH_02) | |

Example nucleotide sequences for arms of homology for certain loci for certain HDR templates, and useful with certain embodiments of the methods and compositions provided herein are listed in TABLE 2.

TABLE 2

| SEQ ID | Nucleic acid/ | |
|---|---|---|
| NO | features | Nucleotide sequence |
| SEQ ID | Emu_HA_L; | TGTGACGCCCGGAGACAGAAGGTCTCTGGGTGGCTG |
| NO: 29 | Left arm for | GGTTTTTGTGGGGTGAGGATGGACATTCTGCCATTGT |
| | Emu sgRNA | GATTACTACTACTACTACTACATGGACGTCTGGGGCA |
| | | AAGGGACCACGGTCACCGTCTCCTCAGGTAAGAATG |
| | | GCCACTCTAGGGCCTTTGTTTTCTGCTACTGCCTGTGG |
| | | GGTTTCCTGAGCATTGCAGGTTGGTCCTCGGGGCATG |
| | | TTCCGAGGGGACCTGGGCGGACTGGCCAGGAGGGGA |
| | | TGGGCACTGGGGTGCCTTGAGGATCTGGGAGCCTCTG |
| | | TGGATTTTCCGATGCCTTTGGAAAATGGGACTCAGGT |
| | | TGGGTGCGTCTGATGGAGTAACTGAGCCTGGGGGCTT |
| | | GGGGAGCCACATTTGGACGAGATGCCTGAACAAACC |
| | | AGGGGTCTTAGTGATGGCTGAGGAATGTGTCTCAGG |
| | | AGCGGTGTCT |
| SEQ ID | Emu_HA_R; | GTAGGACTGCAAGATCGCTGCACAGCAGCGAATCGT |
| NO: 30 | Right arm for | GAAATATTTTCTTTAGAATTATGAGGTGCGCTGTGTG |
| | Emu_sgRNA | TCAACCTGCATCTTAAATTCTTTATTGGCTGGAAAGA |
| | | GAACTGTCGGAGTGGGTGAATCCAGCCAGGAGGGAC |
| | | GCGTAGCCCCGGTCTTGATGAGAGCAGGGTTGGGGG |
| | | CAGGGGTAGCCCAGAAACGGTGGCTGCCGTCCTGAC |
| | | AGGGGCTTAGGGAGGCTCCAGGACCTCAGTGCCTTG |
| | | AAGCTGGTTTCCATGAGAAAAGGATTGTTTATCTTAG |
| | | GAGGCATGCTTACTGTTAAAAGACAGGATATGTTTGA |
| | | AGTGGCTTCTGAGAAAAATGGTTAAGAAAATTATGA |
| | | CTTAAAAATGTGAGAGATTTTCAAGTATATTAATTTT |
| | | TTTAACTGTCCAAGTATTTGAAATTCTTATCATTTGAT |
| | | TAACACCCATG |
| SEQ ID | CCR5_HA_L; | TCAAGTGTCAAGTCCAATCTATGACATCAATTATTAT |
| NO: 31 | Left arm for | ACATCGGAGCCCTGCCAAAAAATCAATGTGAAGCAA |
| | RJ69 | ATCGCAGCCCGCCTCCTGCCTCCGCTCTACTCACTGG |
| | | TGTTCATCTTTGGTTTTGTGGGCAACATGCTGGTCATC |
| | | CTCATCCTGATAAACTGCAAAAGGCTGAAGAGCATG |
| | | ACTGACATCTACCTGCTCAACCTGGCCATCTCTGACC |
| | | TGTTTTTCCTTCTTACTGTCCCCTTCTGGGCTCACTAT |
| | | GCTGCCGCCCAGTGGGACTTTGGAAATACAATGTGTC |
| | | AAC |
| SEQ ID | CCR5_HA_R; | CTCTATTTTATAGGCTTCTTCTCTGGAATCTTCTTCAT |
| NO: 32 | Right arm | CATCCTCCTGACAATCGATAGGTACCTGGCTGTCGTC |
| | for RJ69 | CATGCTGTGTTTGCTTTAAAAGCCAGGACGGTCACCT |
| | | TTGGGGTGGTGACAAGTGTGATCACTTGGGTGGTGGC |
| | | TGTGTTTGCGTCTCTCCCAGGAATCATCTTTACCAGA |
| | | TCTCAAAAAGAAGGTCTTCATTACACCTGCAGCTCTC |
| | | ATTTTCCATACAGTCAGTATCAATTCTGGAAGAATTT |
| | | CCAGACATTAAAGATAGTCATCTTGGGGCTGGTCCTG |
| | | CCG |
| SEQ ID | IGHG1_HA_ | TGGGCTCTGCAGAGAGAAGATTGGGAGTTACTGGAA |
| NO: 33 | L; Left arm | TCTGGGAGGAGAGAAGGTGTCCGAGCTGAGGGAGTG |
| | for RJ178 | GAGAGTTTGGCCTTTGGGGTGGGCTTAGGTCAGGGG |
| | (TH26) | CAGGGTCCTCCCGGATATGGCTCTTGGCAGGTCTGAG |
| | | CCCAGCACCTGCCCCTTTGTGTGCAGGGCCTGGGTTA |
| | | GGGGCACCTAGCCTGTGCCTGCCCAGAGCCTGGGGA |
| | | AAAAGCCAGAAGACCCTCTCCCTGAGCATGAGTGGG |
| | | GCGGGCAGAGGCCTCCGGGTGAAGAGGCAGACGGG |
| | | GCCTGCCT |
| SEQ ID | IGHG1_HA_ | ATCTTGTGACAAAACTCACACATGCCCACCGTGCCCA |
| NO: 34 | R; Right arm | GGTAAGCCAGCCCAGGCCTCGCCCTCCAGCTCAAGG |
| | for RJ178 | CGGGACAGGTGCCCTAGAGTAGCCTGCATCCAGGGA |
| | (TH26) | CAGGCCCCAGCCGGGTGCTGACACGTCCACCTCCATC |
| | | TCTTCCTCAGCACCTGAACTCCTGGGGGGACCGTCAG |
| | | TCTTCCTCTTCCCCCCAAAACCCAAGGACACCCTCAT |
| | | GATCTCCCGGACCCCTGAGGTCACATGCGTGGTGGTG |
| | | GACGTGAGCCACGAAGACCCTGAGGTCAAGTTCAAC |
| | | TGGTACG |
| SEQ ID | JCHAIN_HA_ | TTTATATAGTGTAAATGCAAAATAAAAATGATTGTAT |
| NO: 35 | L; Left arm | CAAATTATGGTTTTTATTTGGATGCTTTGCAAGAAAA |
| | for RJ94 | TACTTAAATGTGAGTCATACATTTTTCCAGTTCAGCA |
| | | TTTAAACTTCAGACTTCAACCACAGTTGTGATTGTTTT |
| | | TAGTTTGTTAGCTGCCTGGAGTGTTATTTTAAGAAAG |
| | | CAGAAGCACCATCATTTGCACACTCCTTATAGATCAC |
| | | ACACCTTAACCCTGACTTTTTTTGCTCCAGTTTTTCAG |
| | | AAGAAGTGAAGTCAAGATGAAGAACCATTTGCTTTT |
| SEQ ID | JCHAIN_HA_ | AGTCCTGGCGGTTTTTATTAAGGCTGTTCATGTGAAA |
| NO: 36 | R; Right arm | GGTATGTGATATTTAGAAAATGATCCCAGATCAAAG |
| | for RJ94 | GAAAAATATAGGACAGATCTGTTTTTCAGTTTTAAAC |
| | | ATTTATGCCTATGCTTTGGTTGGCCAGTTAGCCAGCT |
| | | ACTTTTTCCTAACATGCTTTCATCTTTCTATGGCAGTT |
| | | GTCTTTACATAGATAAGACTCTTTTTATTCTTTTTTCT |
| | | CTTATCTTACTTACTTGTTTGCCTACTACAAACTAGAG |
| | | AATAAGAAATTAGCTAACTACAGATAGTCCTGC |
| SEQ ID | CD19_HA_L; | CAAATAAACACACAAGATCATTTCCCGTGGTAGTGA |
| NO: 37 | Left arm for | GAGCTGGGATGAAAATAAAACAGCGTGGCAGGGAG |
| | RJ255 | GAGGCAAGTGTTGTGAGTCTGGAGGGTTCCTGGAGA |
| | | ATGGGGCCTGAGGCGTGACCACCGCCTTCCTCTCTGG |
| | | GGGGACTGCCTGCCGCCCCCGCAGACACCCATGGTT |
| | | GAGTGCCCTCCAGGCCCCTGCCTGCCCCAGCATCCCC |
| | | TGCGCGAAGCTGGGTGCCCCGGAGAGTCTGACCACC |
| | | ATGCCACCTCCTCGCCTCCTCTTCTTCCTCCTCTTCCT |
| | | CACCCCCATCGAACAGAGAAACAGGAGAATATGGGC |
| | | CAAACAGGATATCTGTGGTAAGCAGTTCCTGCCCCGG |
| | | CTCAGGGCCAAGAACAGATGGTCCCCAGATGCGGTC |
| | | CCGCCCTCAGCAGTTTCT |
| SEQ ID | CD19_HA_R; | GGAAGTCAGGCCCGAGGAACCTCTAGTGGTGAAGGT |
| NO: 38 | Right arm for | GGAAGGTATGTCCAAAGGGCAGAAAGGGAAGGGATT |
| | RJ255 | GAGGCTGGAAACTTGAGTTGTGGCTGGGTGTCCTTGG |
| | | CTGAGTAACTTACCCTCTCTGAGCCTCCATTTTCTTAT |
| | | TTGTAAAATTCAGGAAAGGGTTGGAAGGACTCTGCC |
| | | GGCTCCTCCACTCCCAGCTTTTGGAGTCCTCTGCTCTA |
| | | TAACCTGGTGTGAGGAGTCGGGGGGCTTGGAGGTCC |
| | | CCCCCACCCATGCCCACACCTCTCTCCCTCTCTCTCCA |
| | | CAGAGGGA |
| SEQ ID | IGHG1_HA_ | GCATGAGCCGTCCCTGAGGTGGCACCGATGGCCAGA |
| NO: 39 | L; left arm for | GCTGAGGCCAAGCTAGAGGOCCTGGACTGTGCTGAC |
| | TH16 | TCCCGGCAGACACAGAGCGCTGACCTGGCTGCCGAG |
| | | CCCCGCCTCCTAGGCTGCAGGGGTGCCTGCAGAAGG |
| | | GCACCACAGGGCCACCGGTCCTGCAAGCTTTCTGGG |
| | | GCAGGCCGGGCCTGACCTTGGCTTTGGGGCAGGGGG |
| | | TGGGCTAAGGTGACGCAGGTGGCGCCAGCCAGGCGC |
| | | ACACCCAATGCCCGTGAGCCCAGACACTGGACGCTG |
| | | AACCTCGCGGACAGTTAAGAACCCAGGGGCCTCTGC |
| | | GCCCTGGGCCCAGCTCTGTCCCACACCGCGGTCACAT |
| | | GGCACCACCTCTCTTGCAGCCTCCACCAAGGGCCCAT |
| | | CG |
| SEQ ID | IGHG1_HA_ | GGTGAAGAGGCAGACGGGGCCTGCCTTGCTGCCCTG |
| NO: 40 | R; right arm | GACTGGGGCTGCATAGCCGGGATGCGTCCAGGCAGG |
| | for TH16 | AGCGCTGAGCCTGGCTTCCAGCAGACACCCTCCCTCC |
| | | CTGTGCTGGCCTCTCACCAACTTTCTTGTCCACCTTGG |
| | | TGTTGCTGGGCTTGTGATTCACGTTGCAGATGTAGGT |
| | | CTGGGTGCCCAAGCTGCTGGAGGGCACGGTCACCAC |
| | | GCTGCTGAGGGAGTAGAGTCCTGAGGACTGTAGGAC |
| | | AGCCGGGAAGGTGTGCACGCCGCTGGTCAGGGCGCC |
| | | TGAGTTCCACGACACCGTCACCGGTTCGGGGAAGTA |
| | | GTCCTTGACCAGGCAGCCCAGGGCTGCTGTGCCCCCA |
| | | GAGGTGCTCTTGGAGGAGGGTGCCAGGGGGAAGAC |
| SEQ ID | IGHG1_HA_ | AATGGCAAGGAGTACAAGTGCAAGGTCTCCAACAAA |
| NO: 41 | L; left arm for | GCCCTCCCAGCCCCCATCGAGAAAACCATCTCCAAA |
| | TH21 | GCCAAAGGTGGGACCCGTGGGGTGCGAGGGCCACAT |
| | | GGACAGAGGCCGGCTCGGCCCACCCTCTGCCCTGAG |
| | | AGTGACCGCTGTACCAACCTCTGTCCCTACAGGGCAG |
| | | CCCCGAGAACCACAGGTGTACACCCTGCCCCCATCCC |
| | | GGGATGAGCTGACCAAGAACCAGGTCAGCCTGACCT |
| | | GCCTGGTCAAAGGCTTCTATCCCAGCGACATCGCCGT |
| | | GGAGTGGGAGAGCAATGGGCAGCCGGAGAACAACT |
| | | ACAAGACCACGCCTCCCGTGCTGGACTCCGACGGCTC |
| | | CTTCTTCCTCTACAGCAAGCTCACCGTGGACAAAAGT |
| SEQ ID | IGHG1_HA_ | AGATGGCAGCAGGGGAACGTCTTCTCATGCTCCGTG |
| NO: 42 | R; right arm | ATGCATGAGGCTCTGCACAACCACTACACACAGAAG |
| | for TH21 | AGCCTCTCCCTGTCTCCGGGTAAATGAGTGCCACGGC |
| | | CGGCAAGCCCCCGCTCCCCAGGCTCTCGGGGTCGCGC |
| | | GAGGATGCTTGGCACGTACCCCGTGTACATACTTCCC |
| | | AGGCACCCAGCATGGAAATAAAGCACCCAGCGCTTC |
| | | CCTGGGCCCCTGCGAGACTGTGATGGTTCTTTCCACG |
| | | GGTCAGGCCGAGTCTGAGGCCTGAGTGGCATGAGGG |
| | | AGGCAGAGTGGGTCCCACTGTCCCCACACTGGCCCA |
| | | GGCTGTGCAGGTGTGCCTGGGCCGCCTAGGGTGGGG |
| | | CTCAGCCAGGGGCTGCCCTCGGCAGGGGGGGGATT |
| SEQ ID | IGHG1_HA_ | CGCACACCCAATGCCCGTGAGCCCAGACACTGGACG |
| NO: 43 | L; left arm for | CTGAACCTCGCGGACAGTTAAGAACCCAGGGGCCTC |
| | TH22 | TGCGCCCTGGGCCCAGCTCTGTCCCACACCGCGGTCA |
| | | CATGGCACCACCTCTCTTGCAGCCTCCACCAAGGGCC |
| | | CATCGGTCTTCCCCCTGGCACCCTCCTCCAAGAGCAC |
| | | CTCTGGGGGCACAGCAGCCCTGGGCTGCCTGGTCAA |
| | | GGACTACTTCCCCGAACCGGTGACGGTGTCGTGGAA |
| | | CTCAGGCGCCCTGACCAGCGGCGTGCACACCTTCCCG |
| | | GCTGTCCTACAGTCCTCAGGACTCTACTCCCTCAGCA |
| | | GCGTGGTGACCGTGCCCTCCAGCAGCTTGGGCACCCA |
| | | GACCTACATCTGCAACGTGAATCACAAGCCCAGT |
| SEQ ID | IGHG1_HA_ | AATACTAAGGTGGACAAGAAAGTTGGTGAGAGGCCA |
| NO: 44 | R; right arm | GCACAGGGAGGGAGGGTGTCTGCTGGAAGCCAGGCT |
| | for TH22 | CAGCGCTCCTGCCTGGACGCATCCCGGCTATGCAGCC |
| | | CCAGTCCAGGGCAGCAAGGCAGGCCCCGTCTGCCTC |
| | | TTCACCCGGAGGCCTCTGCCCGCCCCACTCATGCTCA |
| | | GGGAGAGGGTCTTCTGGCTTTTTCCCCAGGCTCTGGG |
| | | CAGGCACAGGCTAGGTGCCCCTAACCCAGGCCCTGC |
| | | ACACAAAGGGGCAGGTGCTGGGCTCAGACCTGCCAA |
| | | GAGCCATATCCGGGAGGACCCTGCCCCTGACCTAAG |
| | | CCCACCCCAAAGGCCAAACTCTCCACTCCCTCAGCTC |
| | | GGACACCTTCTCTCCTCCCAGATTCCAGTAACTCCC |
| SEQ ID | IGHG1_HA_ | CTGAGGTGGCACCGATGGCCAGAGCTGAGGCCAAGC |
| NO: 45 | L; left arm for | TAGAGGCCCTGGACTGTGCTGACTCCCGGCAGACAC |
| | TH27 | AGAGCGCTGACCTGGCTGCCGAGCCCCGCCTCCTAG |
| | | GCTGCAGGGGTGCCTGCAGAAGGGCACCACAGGGCC |
| | | ACCGGTCCTGCAAGCTTTCTGGGGCAGGCCGGGCCTG |
| | | ACCTTGGCTTTGGGGCAGGGGGTGGGCTAAGGTGAC |
| | | GCAGGTGGCGCCAGCCAGGCGCACACCCAATGCCCG |
| | | TGAGCCCAGACACTGGACGCTGAACCTCGCGGACAG |
| | | TTAAGAACCCAGGGGCCTCTGCGCCCTGGGCCCAGCT |
| | | CTGTCCCACACCGCGGTCACATGGCACCACCTCTCTT |
| | | GCAGCCTCCACCAAGGGCCCATCG |
| SEQ ID | IGHG1_HA_ | CACCCTCCTCCAAGAGCACCTCTGGGGGCACAGCAG |
| NO: 46 | R; right arm | CCCTGGGCTGCCTGGTCAAGGACTACTTCCCCGAACC |
| | for TH27 | GGTGACGGTGTCGTGGAACTCAGGCGCCCTGACCAG |
| | | CGGCGTGCACACCTTCCCGGCTGTCCTACAGTCCTCA |
| | | GGACTCTACTCCCTCAGCAGCGTGGTGACCGTGCCCT |
| | | CCAGCAGCTTGGGCACCCAGACCTACATCTGCAACGT |
| | | GAATCACAAGCCCAGCAACACCAAGGGGACAAGAA |
| | | AGTTGGTGAGAGGCCAGCACAGGGAGGGAGGGTGTC |
| | | TGCTGGAAGCCAGGCTCAGCGCTCCTGCCTGGACGC |
| | | ATCCCGGCTATGCAGCCCCAGTCCAGGGCAGCAAGG |
| | | CAGGCCCCGTCTGCCTCTTCACCCGGAGGCCTCTGCC |
| | | CCGCGGTCTAGAGCATGGCTACGTAGAT |
| SEQ ID | IGHG1_HA_ | CCGCCTCCTAGGCTGCAGGGGTGCCTGCAGAAGGGC |
| NO: 47 | L; left arm for | ACCACAGGGCCACCGGTCCTGCAAGCTTTCTGGGGC |
| | TH28 | AGGCCGGGCCTGACCTTGGCTTTGGGGCAGGGGGTG |
| | | GGCTAAGGTGACGCAGGTGGCGCCAGCCAGGCGCAC |
| | | ACCCAATGCCCGTGAGCCCAGACACTGGACGCTGAA |
| | | CCTCGCGGACAGTTAAGAACCCAGGGGCCTCTGCGC |
| | | CCTGGGCCCAGCTCTGTCCCACACCGCGGTCACATGG |
| | | CACCACCTCTCTTGCAGCCTCCACCAAGGGCCCATCG |
| SEQ ID | IGHG1_HA_ | GGTGAAGAGGCAGACGGGGCCTGCCTTGCTGCCCTG |
| NO: 48 | R; right arm | GACTGGGGCTGCATAGCCGGGATGCGTCCAGGCAGG |
| | for TH28 | AGCGCTGAGCCTGGCTTCCAGCAGACACCCTCCCTCC |
| | | CTGTGCTGGCCTCTCACCAACTTTCTTGTCCACCTTGG |
| | | TGTTGCTGGGCTTGTGATTCACGTTGCAGATGTAGGT |
| | | CTGGGTGCCCAAGCTGCTGGAGGGCACGGTCACCAC |
| | | GCTGCTGAGGGAGTAGAGTCCTGAGGACTGTAGGAC |
| | | AGCCGGGAAGGTGTGCACGCCGCTGGTCAGGGCGCC |
| | | TGAGTTCCACGACACCGTCACCGGTTCGGGGAAGTA |
| | | GTCCTTGACCAGGCAGCCCAGGGCTGCTGTGCCCCCA |
| | | GAGGTGCTCTTGGAGGAGGGTG |

Example nucleotide sequences useful with certain embodiments of the methods and compositions provided herein are listed in TABLE 3.

TABLE 3

| SEQ ID | Nucleic acid/ | |
|---|---|---|
| NO | feature | Nucleotide sequence |
| SEQ ID | BTCE | GACATTCAGCTTACGCAGAGCCCCGCGTCTCTTGCAG |
| NO: 49 | blinaumomab | TGAGCCTTGGTCAACGGGCGACAATCTCTTGCAAGGC |
| | | TAGTCAATCTGTAGACTATGACGGAGATTCATATTTG |
| | | AACTGGTATCAGCAAATCCCGGGCCAACCGCCTAAA |
| | | TTGCTCATATACGACGCATCTAACCTGGTCAGCGGGA |
| | | TTCCCCCGCGCTTTTCCGGGTCCGGTTCCGGGACAGA |
| | | TTTCACCCTGAACATCCATCCCGTCGAAAAGGTTGAT |
| | | GCGGCCACTTACCATTGCCAACAGTCCACTGAAGACC |
| | | CCTGGACATTCGGTGGGGGAACTAAGCTCGAAATAA |
| | | AGAGTGGCGGGGGTTCCGGGGGTGGCGGGAGTGGTG |
| | | GTGGCGGAAGTCAAGTCCAATTGCAGCAATCCGGTG |
| | | CTGAACTCGTCCGACCCGGGTCTTCCGTAAAAATTAG |
| | | CTGCAAGGCGAGCGGCTACGCTTTCTCCTCTTATTGG |
| | | ATGAATTGGGTTAAGCAGCGGCCAGGACAGGGACTT |
| | | GAGTGGATAGGCCAAATATGGCCTGGGGATGGTGAC |
| | | ACGAATTACAACGGTAAGTTCAAGGGGAAAGCTACA |
| | | CTCACGGCGGACGAGAGTTCAAGCACGGCGTATATG |
| | | CAGCTCTCATCACTCGCATCAGAAGATAGTGCAGTTT |
| | | ACTTCTGTGCGCGAAGAGAGACAACAACTGTTGGGC |
| | | GCTATTACTATGCGATGGACTATTGGGGCCAGGGGAC |
| | | TACAGTTACGGTCTCATCTGGGGGAGGCGGAAGCGA |
| | | CATAAAGCTACAGCAAAGTGGTGCAGAGCTTGCGCG |
| | | GCCGGGTGCCTCTGTTAAGATGAGTTGCAAGACATCC |
| | | GGATATACTTTCACCAGGTACACAATGCATTGGGTTA |
| | | AACAGAGACCCGGACAGGGGTTGGAATGGATTGGTT |
| | | ACATAAATCCCTCCAGGGGTTATACTAACTACAATCA |
| | | GAAATTCAAAGACAAAGCAACCTTGACTACGGACAA |
| | | AAGCTCAAGCACTGCGTATATGCAACTTTCAAGCCTT |
| | | ACAAGCGAAGATTCAGCGGTGTACTATTGTGCACGCT |
| | | ATTACGACGATCACTACTGTCTGGACTACTGGGGGCA |
| | | AGGTACTACGCTGACCGTCAGCTCCGTGGAGGGGGG |
| | | TTCTGGTGGGAGTGGTGGAAGCGGCGGAAGTGGTGG |
| | | AGTGGACGATATACAGTTGACGCAAAGCCCAGCGAT |
| | | CATGTCTGCTTCTCCTGGAGAAAAGGTTACGATGACA |
| | | TGCAGAGCTAGTAGCTCTGTCAGTTATATGAATTGGT |
| | | ATCAGCAGAAAAGCGGGACGAGTCCGAAACGGTGGA |
| | | TATATGATACCTCCAAAGTAGCCTCTGGAGTACCGTA |
| | | CCGATTTTCTGGAAGCGGCTCAGGAACTAGCTACTCC |
| | | CTTACGATAAGCAGTATGGAGGCAGAAGACGCTGCC |
| | | ACATATTACTGTCAACAGTGGTCCAGCAACCCCCTTA |
| | | CATTTGGTGCTGGTACGAAGTTGGAGTTGAAGCATCA |
| | | TCATCATCACCAC |
| SEQ ID | BTCE | ATGCAGGTTCAACTCGTTCAGAGTGGTGCCGAGGTTA |
| NO: 50 | AMG-330 | AAAAACCGGGGGAATCCGTCAAAGTAAGCTGCAAGG |
| | | CATCCGGGTATACGTTTACGAACTACGGGATGAACTG |
| | | GGTCAAGCAAGCGCCCGGGCAAGGCTTGGAATGGAT |
| | | GGGCTGGATCAACACCTACACAGGTGAGCCGACCTA |
| | | CGCTGATAAGTTCCAAGGACGAGTAACGATGACAAC |
| | | GGATACGTCAACATCAACGGCCTATATGGAGATACG |
| | | AAACTTGGGTGGGGACGACACAGCAGTCTACTATTG |
| | | CGCTAGGTGGAGCTGGTCTGATGGCTACTACGTATAC |
| | | TTCGATTACTGGGGTCAAGGCACCTCCGTCACGGTCT |
| | | CTTCCGGGGGTGGGGGTTCAGGAGGAGGGGGCTCCG |
| | | GGGGTGGTGGATCAGATATTGTGATGACTCAGTCCCC |
| | | AGATTCATTGACCGTCTCACTGGGAGAACGGACTACG |
| | | ATAAATTGTAAATCTAGCCAAAGTGTACTTGATTCTT |
| | | CAACGAATAAAAATTCTCTTGCGTGGTACCAACAGA |
| | | AACCCGGTCAACCCCCGAAACTCCTCTTGTCCTGGGC |
| | | GTCCACACGGGAATCTGGTATCCCGGATCGGTTCAGT |
| | | GGCTCAGGATCAGGGACAGATTTTACTCTTACAATCG |
| | | ATAGTCCCCAGCCTGAGGACTCAGCGACCTACTACTG |
| | | TCAGCAGTCCGCGCACTTTCCAATTACGTTTGGCCAG |
| | | GGTACGAGGCTCGAGATCAAAAGCGGAGGTGGGGGT |
| | | AGCGAGGTGCAGTTGGTGGAGTCTGGAGGCGGCCTC |
| | | GTTCAACCCGGTGGGTCATTGAAACTTTCATGTGCTG |
| | | CGTCCGGATTTACTTTTAACAAGTACGCTATGAATTG |
| | | GGTGCGACAAGCACCGGGTAAAGGGCTGGAATGGGT |
| | | GGCACGGATAAGGTCTAAATACAATAATTATGCTAC |
| | | GTACTATGCAGATAGTGTAAAGGATAGGTTCACAAT |
| | | AAGTAGGGACGATAGTAAAAATACTGCGTATCTGCA |
| | | GATGAATAATTTGAAAACAGAGGATACAGCGGTCTA |
| | | CTACTGTGTTAGGCATGGGAATTTCGGCAATTCTTAC |
| | | ATATCTTATTGGGCGTATTGGGGACAAGGCACACTTG |
| | | TAACTGTCAGTAGCGGTGGAGGTGGTAGCGGAGGCG |
| | | GCGGGAGTGGAGGTGGGGGATCACAAACGGTTGTGA |
| | | CTCAGGAACCAAGCCTTACAGTCTCTCCAGGAGGCAC |
| | | CGTAACACTGACTTGCGGCTCAAGTACTGGTGCCGTA |
| | | ACTTCTGGCAATTATCCTAACTGGGTCCAGCAGAAAC |
| | | CAGGTCAGGCACCCCGGGGACTGATAGGCGGCACAA |
| | | AGTTTCTGGCACCGGGCACCCCCGCACGCTTTTCTGG |
| | | ATCACTTCTCGGAGGCAAAGCAGCCCTCACCTTGAGC |
| | | GGCGTACAACCAGAAGACGAGGCTGAATACTATTGC |
| | | GTTCTTTGGTACAGTAACCGATGGGTTTTCGGAGGGG |
| | | GTACGAAACTCACAGTGCTTCATCACCACCATCACCA |
| | | C |
| SEQ ID | Promoter | GAACAGAGAAACAGGAGAATATGGGCCAAACAGGA |
| NO: 51 | MND | TATCTGTGGTAAGCAGTTCCTGCCCCGGCTCAGGGCC |
| | | AAGAACAGTTGGAACAGCAGAATATGGGCCAAACAG |
| | | GATATCTGTGGTAAGCAGTTCCTGCCCCGGCTCAGGG |
| | | CCAAGAACAGATGGTCCCCAGATGCGGTCCCGCCCTC |
| | | AGCAGTTTCTAGAGAACCATCAGATGTTTCCAGGGTG |
| | | CCCCAAGGACCTGAAATGACCCTGTGCCTTATTTGAA |
| | | CTAACCAATCAGTTCGCTTCTCGCTTCTGTTCGCGCGC |
| | | TTCTGCTCCCCGAGCTCTATATAAGCAGAGCTCGTTT |
| | | AGTGAACCGTCAGATC |
| SEQ ID | Promoter | GTAATCTTTAGGCCAATAAAATGTGGGTTCACAGTGA |
| NO: 52 | Igh1-69 | GGAGTGCATCCTGGGGTTGGGGTTTGTTCTGCAGCGG |
| | | GAAGAGCGCTGTGCACAGAAAGCTTAGAAATGGGGC |
| | | AAGAGATGCTTTTCCTCAGGCAGGATTTAGGGCTTGG |
| | | TCTCTCAGCATCCCACACTTGTACAGCTGATGTGGCA |
| | | TCTGTGTTTTCTTTCTCATCCTAGATCAGGCTTTGAGC |
| | | TGTGAAATACCCTGCCTCATGCATATGCAAATAACCT |
| | | GAGGTCTTCTGAGATAAATATAGATATATTGGTGCCC |
| | | TGAG |
| SEQ ID | Promoter | ACTAGAGGGTATATAATGGAAGCTCGACTTCCAGCTT |
| NO: 53 | minP | GGCAATCCGGTACTGTA |
| SEQ ID | Enhancer | ATGCGGGACTGCGTTTTGACCATCATAAATCAAGTTT |
| NO: 54 | Emu | ATTTTTTTAATTAATTGAGCGAAGCTGGAAGCAGATG |
| | | ATGAATTAGAGTCAAGATGGCTGCATGGGGGTCTCC |
| | | GGCACCCACAGCAGGTGGCAGGAAGCAGGTCACCGC |
| | | GAGAGTCTATTTTAGGAAGCAAAAAAACACAATTGG |
| | | TAAATTTATCACTTCTGGTTGTGAAGAGGTGGTTTTG |
| | | CCCAGGCCCAGATCTGAAAGTGCTCTACTGAGCAAA |
| | | ACAACACCTGGACAATTTGCGTTTCTAAAATAAGGCG |
| | | AGGCTGACCGAAACTGAAAAGGCTTTTTTTAACTATC |
| | | TGAATTTCATTTCCAATCTT |
| SEQ ID | Enhancer | GGTCCAATTCACAAGAACCAGAAGGATGATGTCGCT |
| NO: 55 | SLC3A2_5 | CAGACTGACTTGCTGCAGATCGACCCCAATTTTGGCT |
| | | CCAAGGAAGATTTTGACAGTCTCTTGCAATCGGCTAA |
| | | AAAAAAGAGTGGGTATCCTGGGGTTCCCAAGGAAAC |
| | | AGCTAGAAAGGA |
| SEQ ID | Enhancer | ACCGAGGTGGATATGAAGGAGGTGGAGCTGAATGAG |
| NO: 56 | SLC3A2_3 | TTAGAGCCCGAGAAGCAGCCGATGAACGCGGCGTCT |
| | | GGGGCGGCCATGTCCCTGGCGGGAGCCGAGAAGAAT |
| | | GGTCTGGTGAAGATCAAGGTGGCGGAAGACGAGGCG |
| | | GAGGCGGCA |
| SEQ ID | 5′ UTR | GGTGATGAGCTGTGCTCCCAGGGGCTTCTTCAGAGGA |
| NO: 57 | Igh1-69_utr | GGAATGTGGTTGTTATGTGATGCT |
| SEQ ID | regulatory | ATGGACTGGACCTGGAGGTTCCTCTTTGTGGTGGCAG |
| NO: 58 | sequence_signal | CAGCTACAGGTAAGGGGCTTCCTAGTCCTAAGGCTGA |
| | sequence | GGAAGGGATCCTGGTTTAGTTAAAGAGGATTTTATTC |
| | Igh1- | ACCCCTGTGTCCTCTCCACAGGTGTCCAGTCC |
| | 69_ex1_intron_ | |
| | ex2 | |
| SEQ ID | 5′ UTR | AGCTCTGGGAGAGGAGCCCCAGCCGTGAGATTCCCA |
| NO: 59 | Ighv3-49_utr | GGAGTTTCCACTTGGTGATCAGCACTGAACACAGACC |
| | | ACCAACC |
| SEQ ID | regulatory | ATGGAGTTTGGGCTTAGCTGGGTTTTCCTTGTTGCTAT |
| NO: 60 | sequence_signal | TTTAAAAGGTAATTCATGGTGTACTAGAGATACTGAG |
| | sequence | TGTGAGGGGACATGAGTGGTAGAAACAGTGGATATG |
| | Ighv3- | TGTGGCAGTTTCTGACCTTGGTGTTTCTGTGTTTGCAG |
| | 49_ex1_intron_ | GTGTCCAATGT |
| | ex2 | |
| SEQ ID | 3′ UTR | GTGCCACGGCCGGCAAGCCCCCGCTCCCCAGGCTCTC |
| NO: 61 | IGHG1_utr | GGGGTCGCGCGAGGATGCTTGGCACGTACCCCGTGT |
| | | ACATACTTCCCAGGCACCCAGCATGGAAATAAAGCA |
| | | CCCAGCGCTTCCCTGGGCCCCTGCGA |
| SEQ ID | SV40 polyA | TGCTTTATTTGTGAAATTTGTGATGCTATTGCTTTATT |
| NO: 62 | | TGTAACCATTATAAGCTGCAATAAACAAGTTAACAAC |
| | | AACAATTGCATTCATTTTATGTTTCAGGTTCAGGGGG |
| | | AGATGTGGGAGGTTTTTTAAAGC |
| SEQ ID | WPRE | CCACGGAATTGTCAGTGCCCAACAGCCGAGCCCCTGT |
| NO: 63 | | CCAGCAGCGGGCAAGGCAGGCGGCGATGAGTTCCGC |
| | | CGTGGCAAGAACTAACCAGGATTTATACAAGGAGGA |
| | | GAAAATGAAAGCCATACGGGAAGCAATAGCATGATA |
| | | CAAAGGCATTAAAGCAGCGTATCCACATAGCGTAAA |
| | | AGGAGCAACATAGTTAAGAATACCAGTCAATCTTTCA |
| | | CAAATTTTGTAATCCAGAGGTTGATTATC |
| SEQ ID | P2A | AGGGCAAAGAGGGGATCAGGAGCTACGAATTTTTCT |
| NO: 64 | | TTATTAAAACAAGCAGGAGATGTTGAGGAGAATCCC |
| | | GGACCG |
| SEQ ID | Reporter | ATGGTGAGCAAGGGCGAGGAGCTGTTCACCGGGGTG |
| NO: 65 | GFP | GTGCCCATCCTGGTCGAGCTGGACGGCGACGTAAAC |
| | | GGCCACAAGTTCAGCGTGTCCGGCGAGGGCGAGGGC |
| | | GATGCCACCTACGGCAAGCTGACCCTGAAGTTCATCT |
| | | GCACCACCGGCAAGCTGCCCGTGCCCTGGCCCACCCT |
| | | CGTGACCACCCTGACCTACGGCGTGCAGTGCTTCAGC |
| | | CGCTACCCCGACCACATGAAGCAGCACGACTTCTTCA |
| | | AGTCCGCCATGCCCGAAGGCTACGTCCAGGAGCGCA |
| | | CCATCTTCTTCAAGGACGACGGCAACTACAAGACCCG |
| | | CGCCGAGGTGAAGTTCGAGGGCGACACCCTGGTGAA |
| | | CCGCATCGAGCTGAAGGGCATCGACTTCAAGGAGGA |
| | | CGGCAACATCCTGGGGCACAAGCTGGAGTACAACTA |
| | | CAACAGCCACAACGTCTATATCATGGCCGACAAGCA |
| | | GAAGAACGGCATCAAGGTGAACTTCAAGATCCGCCA |
| | | CAACATCGAGGACGGCAGCGTGCAGCTCGCCGACCA |
| | | CTACCAGCAGAACACCCCCATCGGCGACGGCCCCGT |
| | | GCTGCTGCCCGACAACCACTACCTGAGCACCCAGTCC |
| | | GCCCTGAGCAAAGACCCCAACGAGAAGCGCGATCAC |
| | | ATGGTCCTGCTGGAGTTCGTGACCGCCGCCGGGATCA |
| | | CTCTCGGCATGGACGAGCTGTACAAGTAA |
| SEQ ID | signal peptide | ATGGCTACCGGCAGCAGAACAAGCCTGCTGCTCGCTT |
| NO: 66 | | TTGGACTGCTCTGTCTCCCCTGGTTGCAAGAAGGCAG |
| | | CGCC |

### EXAMPLES

Example 1-Engineering Plasma Cells to Secrete Anti-Cancer Biologics in Humanized Mice

Advances in genome-engineering have enabled the generation of plasma cells (PCs) that secrete large quantities of therapeutic proteins. However, in vivo modeling of unmanipulated and engineered human PCs has largely been limited to short-term studies following adoptive transfer of PCs into immunodeficient mouse models including NSG mice. Human PCs are challenging to study in immunodeficient mouse models, in part, due to deficiencies, such as missing or lack of cross-species reactivity, in key factors provided by human bone marrow stromal and myeloid cells. In this example, a hypothesis was tested that immunodeficient mice humanized by engraftment of human CD34+ hematopoietic peripheral stem cells (NSG-huCD34) would increase the engraftment potential of genome-engineered human PCs. Further, it was predicted that this approach might help to elucidate critical interactions between human PCs and human host BM-resident cells (myeloid cells and lymphocytes). Consistent with this concept, autologous edited plasma cells engrafted more efficiently in NSG-huCD34 mice than in NSG control mice. Further, gene-edited PCs in NSG-huCD34 mice secreted substantially higher levels of antibodies (>100 μg/mL for over 80 days) and engrafted in additional lymphoid compartments. Gene-edited PCs migrated to and localized in the bone marrow and spleen within 2 days of transfer and were retained in these locations based upon luciferase-based in vivo imaging, ex vivo flow analysis and histopathology. Taken together, these data demonstrated that the presence of autologous human hematopoietic cells in NSG-huCD34 mice achieved establishment of a robust model for studying the in vivo biology of gene engineered, long-lived human PCs.

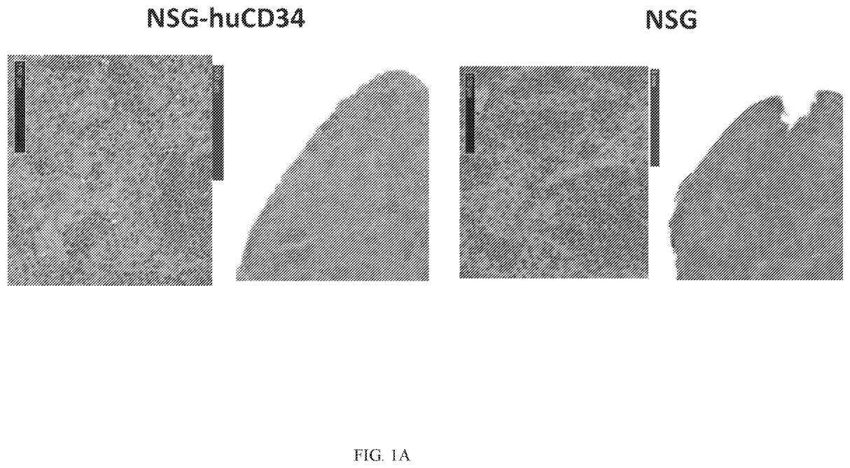

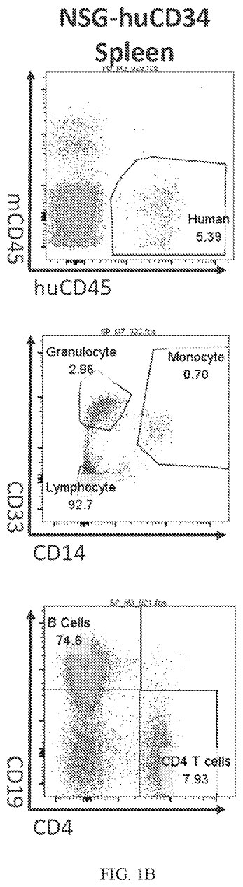

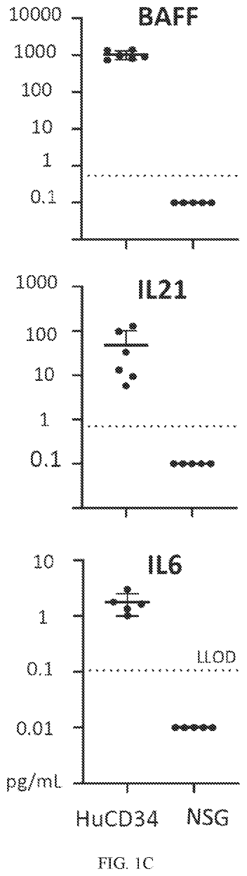

A characterization of NSG-huCD34 mice was performed which included hematoxylin and eosin staining of spleen sections from NSG and NSG-huCD34 (FIG. 1A); flow cytometry from NSGhuCD34 spleen (FIG. 1B), and measuring plasma cell cytokines in peripheral sera of NSG-huCD34 and NSG (FIG. 1C).

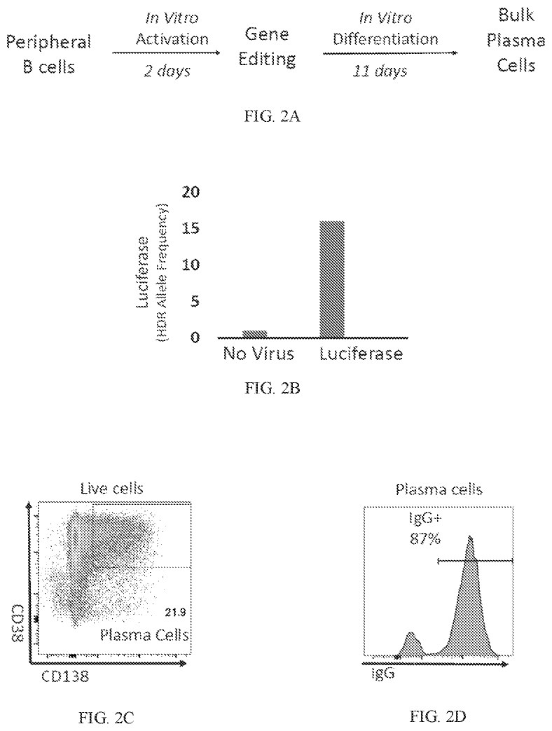

Primary gene-edited in vitro differentiated plasma cells that expressed luciferase derived from primary human B cells were generated according to an in vitro timeline (FIG. 2A). The frequency of HDR edited alleles in primary B cells edited with and without luciferase vector were measured (FIG. 2B). A flow cytometry analyses were performed for plasma cell staining (CD138+CD38+) of live cells 11 days after gene-editing (FIG. 2C), and for IgG staining of plasma cells (FIG. 2D).

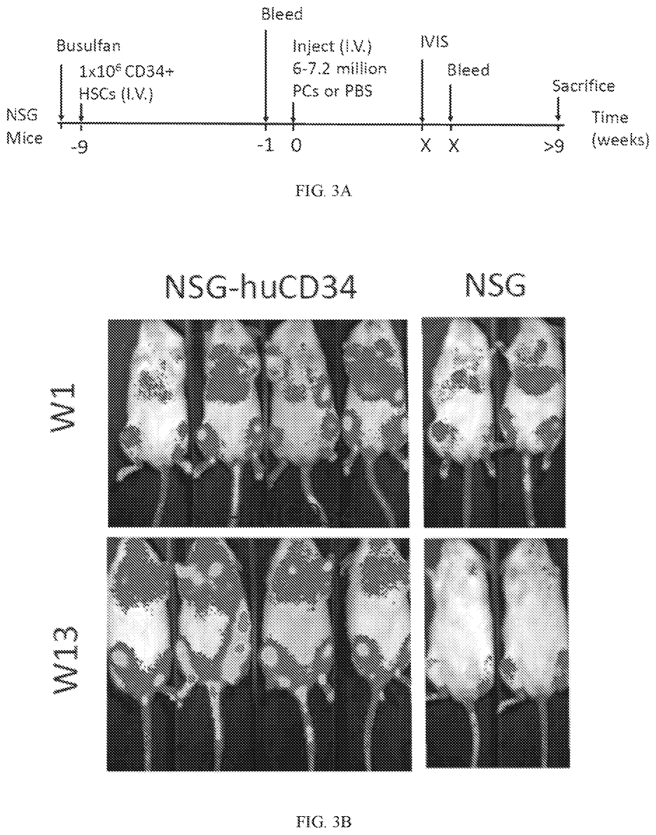

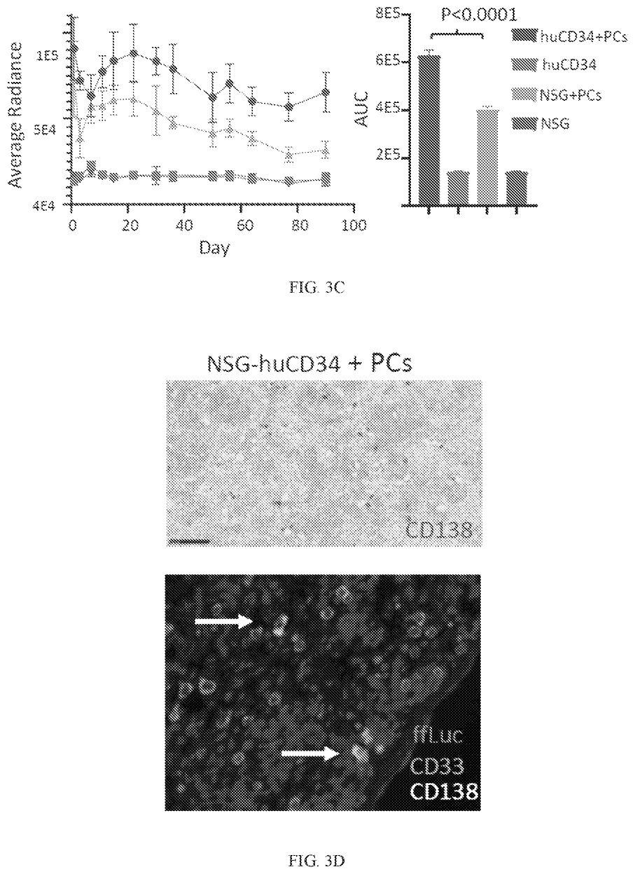

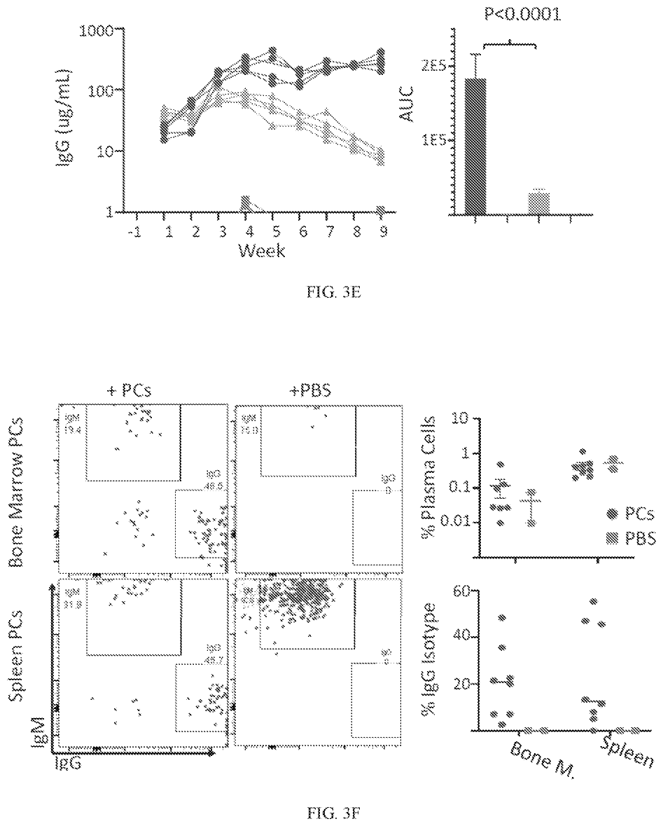

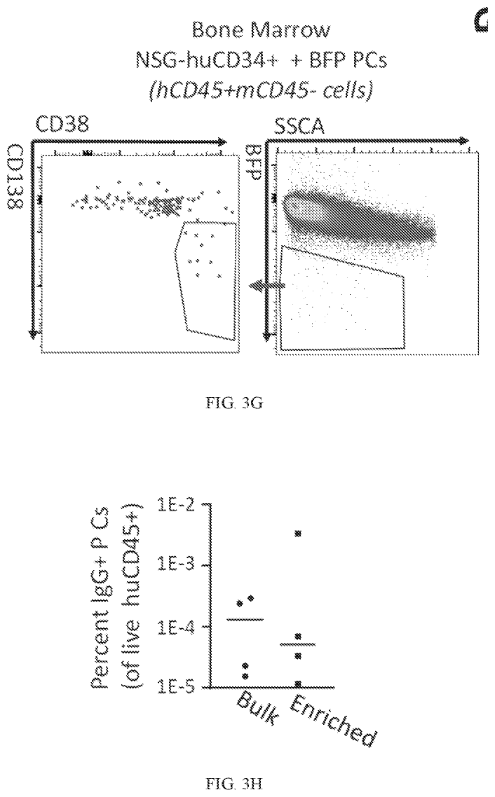

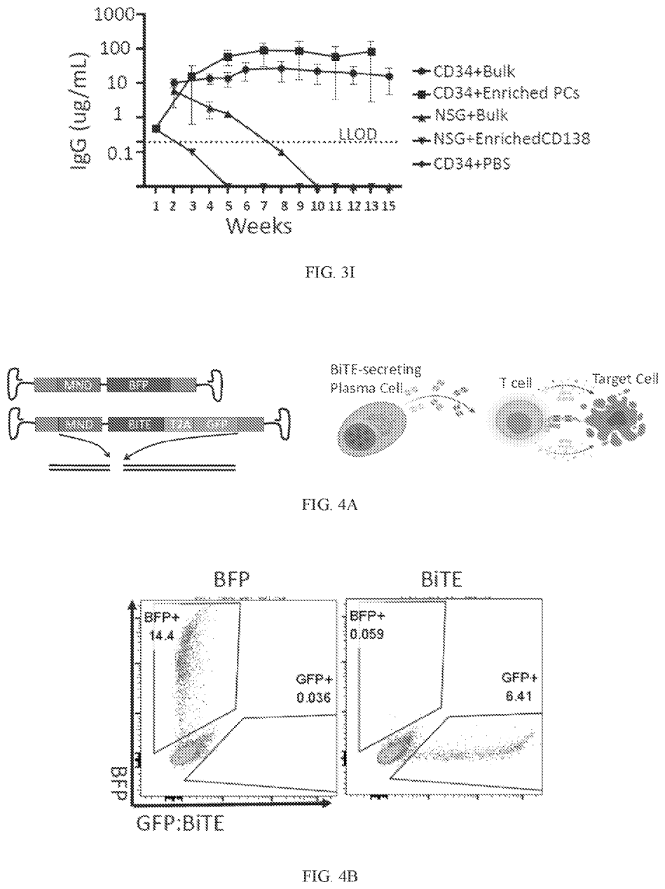

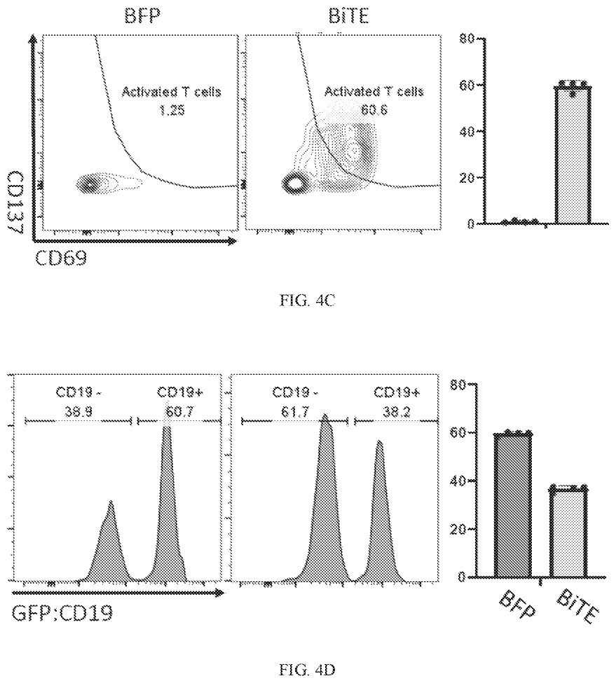

Engraftment of gene-edited human plasma cells in NSG-huCD34 was performed consistent with a timeline for in vivo studies in which NSG and NSG-huCD34 mice received gene-edited plasma cells (PCs) or PBS control (FIG. 3A). Luminescence images of NSG and NSG-huCD34 mice engrafted with bulk plasma cells showed engraftment of ffluc-PCs (FIG. 3B). Average radiance of NSG and NSG-huCD34 mice engrafted with ffluc-PCs showed improved engraftment and persistence in humanized mice (FIG. 3C). An immunohistology of spleen from NSG-huCD34 with ffLuc-PCs was performed (FIG. 3D). Serum IgG titers in NSG and NSG-huCD34 mice that received bulk plasma cells were measured (FIG. 3E). Flow plots of intracellular IgM and IgG of plasma cells (CD138+, CD38+,hCD45+) showed IgG+ cells in mice that received edited PCs (FIG. 3F). PCs edited to express BFP were detected in the bone marrow of NSG-huCD34 mice (FIG. 3G). NSG and NSG-huCD34 mice were engrafted with either 6×10⁶ bulk edited cells or 1×10⁶ enriched ffLuc-plasma cells. Bulk and enriched groups showed engraftment of (FIG. 3H) IgG+ PCs in bone marrow and similar (FIG. 3I) IgG titers. Bispecific T cell Engager (BiTE or BTCE) secreting plasma cells were generated (FIG. 4A) Flow analyses were performed with edited PCs (FIG. 4B). T cells, K562, K652-CD19+-GFP cells were cultured with supernatants from PCs (FIG. 4C). Activation of T cells (FIG. 4C) and Killing of K562-CD19+ cells (FIG. 4D) in the presence of BiTE-PC or BFP-PCs supernatant were measured.

From the foregoing, autologous, HDR gene-edited human plasma cells trafficked to the bone marrow in NSG-huCD34 recipient mice as shown by at least the PCs tracking by luminescence; and maintained PC phenotype based on flow cytometry.

Autologous HDR gene-edited human plasma cells engrafted more efficiently in NSG-huCD34 vs. NSG recipient mice as shown by at least stable radiance in humanized mice for over 100 days.

NSG-huCD34 mice engrafted with plasma cells maintain high titers of IgG. Titers exceeded 50 μg/mL in three mice and remained stable for over 100 days.

$$\# of plasma cells engrafted = \frac{( 1 \times {10}^{8} pg / mL * 0.693 * 2.2 mL )}{6.6 days * 100 pg / cellday} \approx 254 , 000 cells$$

Anti-CD19 BiTE or BTCE were observed to be produced by edited PCs at roughly 62 pg/cellday.

### Example 2—Editing IGHG1 Locus

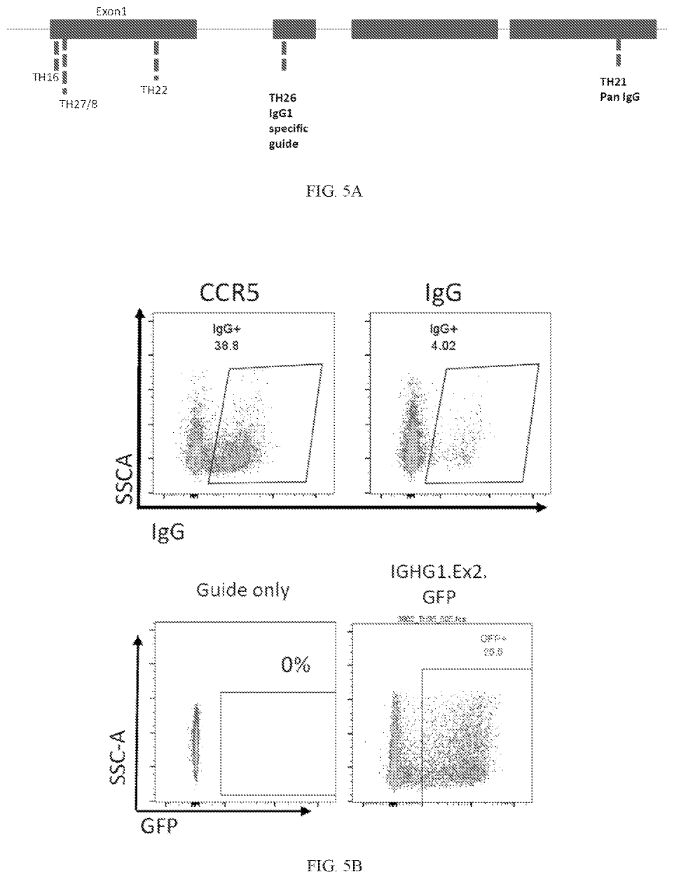

sgRNAs were designed to target several locations in exons 1, 2 and 4 of IGHG1. AAV-based homology-direct repair (HDR) constructs were built for each individual sgRNA and included an MND promoter linked to a GFP reporter gene and located within appropriate homology arms. Target sites for each sgRNA were plotted onto the IGHG1 genomic structure are shown in FIG. 5A. Sequences for sgRNAs and arms of homology for repair templates are listed in TABLE 4.

TABLE 4

| | | sgRNA | Left arm | Right arm |
|---|---|---|---|---|
| Target | | nucleotide | nucleotide | nucleotide |
| site | sgRNA | sequence | sequence | sequence |
| TH16 | sg174 | SEQ ID | SEQ ID NO: 39 | SEQ ID NO: 40 |
| | | NO: 12 | | |
| TH21 | sg193 | SEQ ID | SEQ ID NO: 41 | SEQ ID NO: 42 |
| | | NO: 15 | | |
| TH22 | sgYH_02 | SEQ ID | SEQ ID NO: 43 | SEQ ID NO: 44 |
| | | NO: 28 | | |
| TH26 | sg178 | SEQ ID | SEQ ID NO: 33 | SEQ ID NO: 34 |
| | | NO: 02 | | |
| TH27 | sg173 | SEQ ID | SEQ ID NO: 45 | SEQ ID NO: 46 |
| | | NO: 11 | | |
| TH28 | sg173 | SEQ ID | SEQ ID NO: 47 | SEQ ID NO: 48 |
| | | NO: 11 | | |

B cells were edited with the sgRNA and HDR constructs using methods substantially similar to those described in U.S. 2018/0282692, which is expressly incorporated by reference herein in its entirety.

Briefly, B cells were isolated using negative selection from peripheral blood mononuclear cells (PBMCs), and the B cells were activated using a cytokine cocktail including oligomerized CD40L, CpG and IL21. Following activation the cells were edited: ribonucleoprotein particles including an sgRNA and Cas9 protein were transfected into the cells, and an AAV6 vector containing an HDR construct were introduced into the cells. Following editing, the cells were expanded in activation media for 5 days and switched to 2 stages of differentiation media. A flow cytometry analysis was performed and knockout efficiency of IGHG1 was measured. As shown in FIG. 5B (top panels), a ˜90% decrease in IGHG+ cells in B cells was observed in cells subjected to treatment with RNPs containing TH26 sgRNA. The GFP+ percentage in B cells engineered to express GFP at the IGHG1 locus was measured (FIG. 5B, bottom panels). For live cells, the percentage GFP and mean fluorescence intensity (MFI) were measured, and HDR rates for each set of sgRNA/HDR construct were normalized to those observed with the sgRNA, TH22, in which levels between 10-15% GFP+ cells were typically observed. TABLE 5 lists normalized % GFP positive cells. TABLE 6 lists normalized MFI GFP.

TABLE 5

| | Normalized % GFP positive cells for target sites | | | | | |
|---|---|---|---|---|---|---|
| Subject | TH16 | TH21 | TH22 | TH26 | TH27 | TH28 |
| S1 | 0.74 | 1.54 | 1.00 | 1.77 | 0.56 | 0.63 |
| S2 | 0.95 | 1.54 | 1.00 | | | |
| S3 | 1.03 | 1.30 | 1.00 | 1.53 | 0.94 | 1.20 |

TABLE 6

Normalized MFI GFP for target sites

| Subject | TH16 | TH21 | TH22 | TH26 | TH27 | TH28 |
|---|---|---|---|---|---|---|
| S1 | 1.02 | 5.71 | 1.00 | 0.84 | 0.55 | 0.45 |
| S2 | 0.86 | 4.80 | 1.00 | | | |
| S3 | 0.93 | 6.05 | 1.00 | 1.03 | 0.75 | 0.67 |

In this experiment, targeting the 4^{th} exon (TH21) and the 2^{nd} exon (TH26) led to higher percent transduction and higher MFI.

### Example 3—In Vitro Expression of BTCEs

Expression vectors encoding an anti-CD19 BTCE (Blinotumomab) or (anti-CD33 BTCE (AMG-330) were transfected into 293T cells. Supernatants were obtained, and expression of the BTCEs was quantified using Western blot analysis and mass spectrometry. As expected, both BTCEs produced a 50 KDa protein band on a Western blot, and produced peptides that mapped to the expected sequence.

### Example 4—Editing Eμ Locus

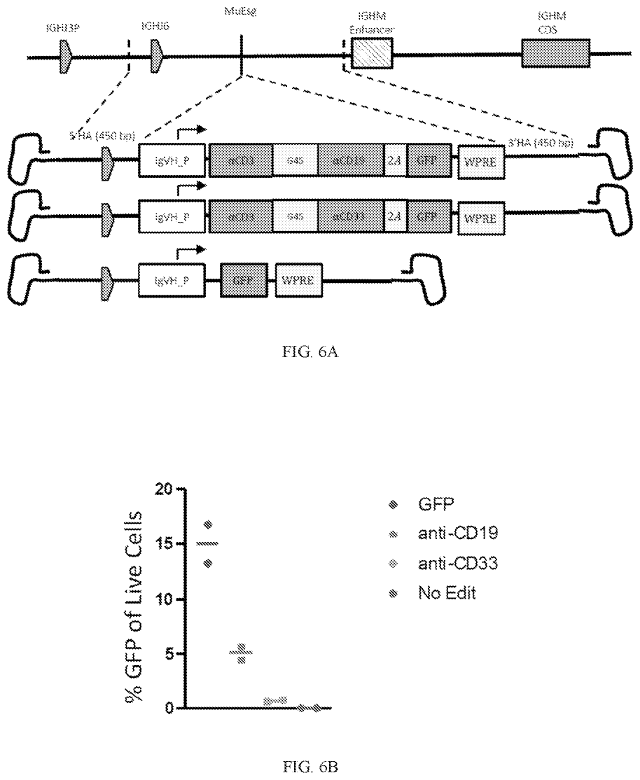

AAV-based HDR repair templates were built to deliver blinatumomab (anti-CD19) or AMG-330 (anti-cd33) to the Emu locus (FIG. 6A).

TABLE 7

| sgRNA | Nucleotide sequence |
|---|---|
| Eμ sgRNA | SEQ ID NO: 01 |

To test the relative editing of each BTCE, B cells were engineered using methods similar to those described above, and described in U.S. 2018/0282692, which is expressly incorporated by reference herein in its entirety. GFP percentage and MFI was quantified as a proportion of live B cells (FIG. 6B).

### Example 5—In Vitro Activity of BTCE-Expressing B Cells

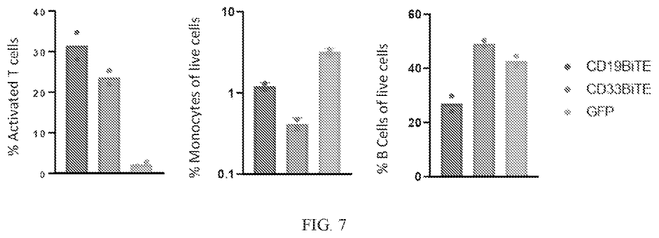

To assay the effect of each BTCE, supernatants from the cells expressing the BTCEs were incubated with autologous PBMCs which contain effector T cells, CD33+ monocytes and CD19+ B cells. After incubation for 48 hours, the effect of each BTCE was quantified by measuring T cell activation (CD69+CD137+ of CD3+ T cells), CD33+ monocyte killing and CD19+B cell killing (FIG. 7). In FIG. 7, left most bars relate to CD19BiTe; middle bars relate to CD33BiTE, and right most bars relate to GFP. As expected, both BTCEs increased T cell activation (left panel). Additionally, in cells incubated with supernatants from B cells producing AMG330, CD33+ depletion was observed (middle bar, middle panel), and in cell incubated with supernatants from B cells producing blinatumomab, we saw CD19+ depletion was observed (left bar, right panel). These data showed efficacy of 2 different BTCEs when secreted by B cells.

### Example 6—Editing Eμ and CCR5 Loci

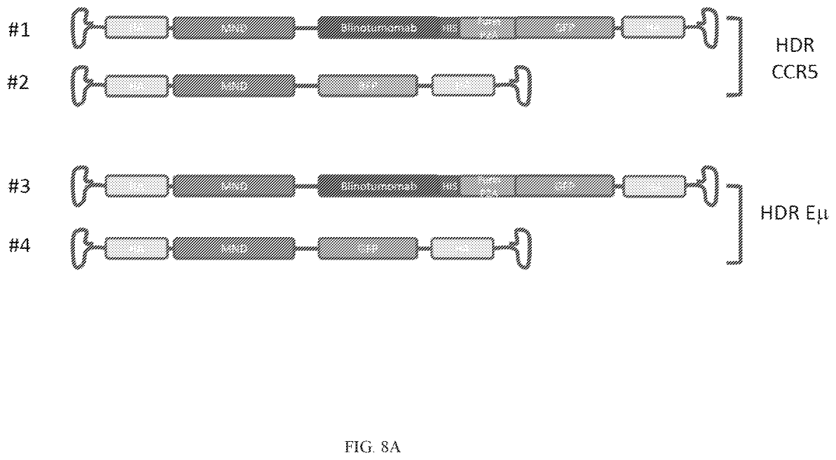

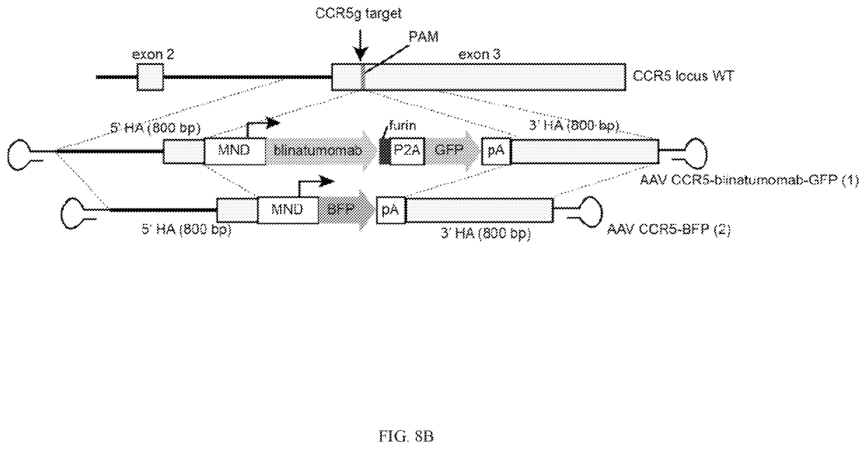

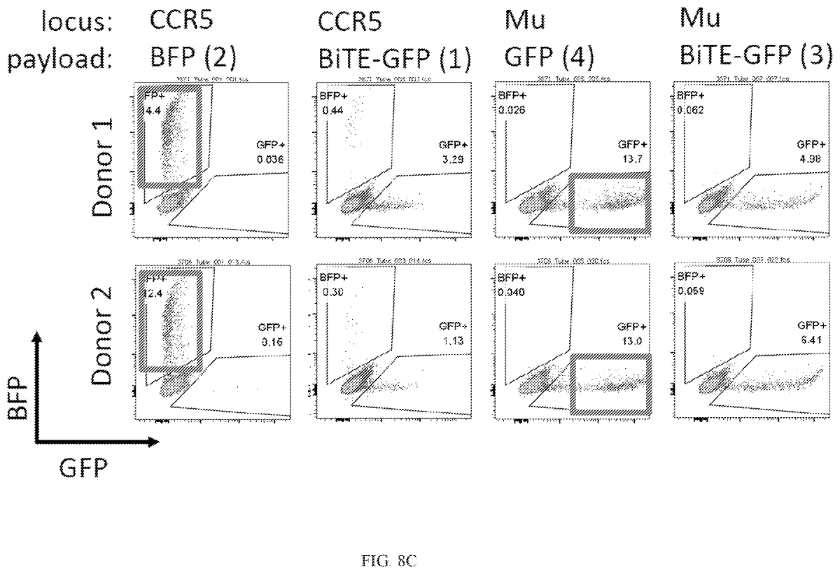

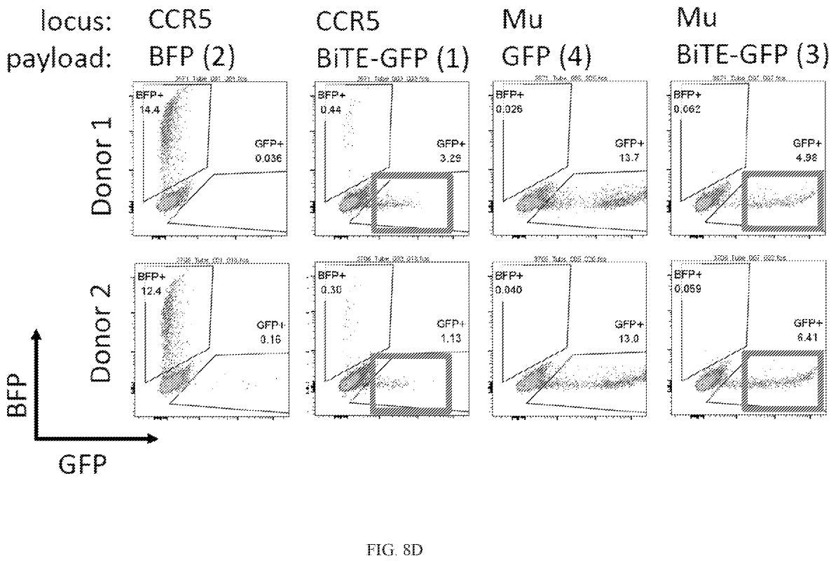

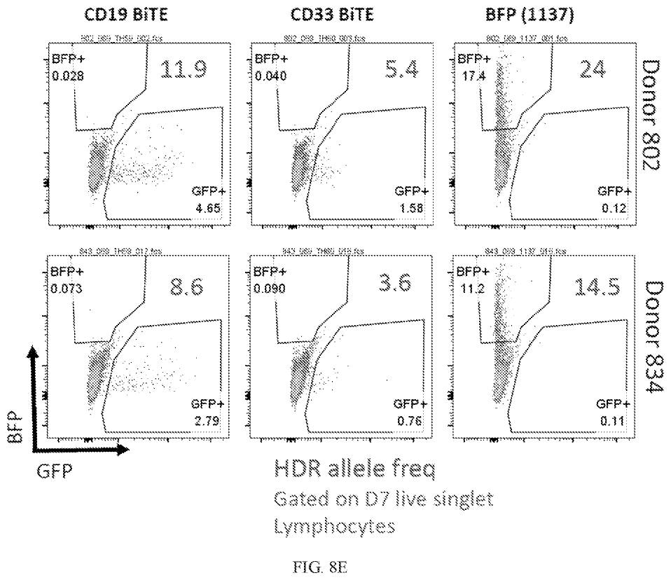

As shown in FIG. 8A, HDR templates were cloned into AAV vectors. Constructs 1 and 2 had homology arms for delivery to the CCR5 locus. Constructs 3 and 4 had homology arms for delivery to the Eμ locus. Constructs 2 and 4 were negative controls that expressed fluorescent markers (BFP in construct 2, GFP in construct 4) downstream of an MND promoter. Constructs 1 and 3 expressed the Blinatumomab BTCE in cis with GFP, also downstream of an MND promoter. In all cases, successful HDR would result in expression of the fluorescent reporter and was predicted to be quantifiable using an in-house digital droplet PCR assay.

B cells were engineered using constructs 1, 2, 3 and 4. FIG. 8B shows constructs used for editing the CCR5 locus. TABLE 8 lists sgRNA sequences used.

TABLE 8

| sgRNA | Nucleotide sequence |
|---|---|
| Eμ sgRNA | SEQ ID NO: 01 |
| CCR5 sgRNA | SEQ ID NO: 22 |

After engineering the B cells, the cells were analyzed to determine the degree of editing and the expression of the fluorescent reporters. Expression of the fluorescent reporters was quantified using a FACS analysis. As shown in FIG. 8C, the highlighted boxes indicate the expression of the fluorescent reporters in the absence of blinatumobab. The mean fluorescent intensity of the fluorescent reporters was similar at both Mu and CCR5 loci. As shown in FIG. 8D, the highlighted boxes indicate expression of the fluorescent reporter as expressed downstream of the blinatumomab. GFP detection as a percentage of total cells, and mean fluorescent intensity was substantially greater in edits delivered to the Eμ locus relative to CCR5.

In another study, B cells were isolated and edited to express blinumomab (anti-cd19 BTCE) was compared to AMG-330 (anti-CD33 BTCE) and BFP when delivered to the CCR5 locus. Similar to the prior study, the proportion of BFP positive cells and BFP's mean fluorescent intensity were both greater than the GFP expressed downstream of either BTCE. To determine whether this deficiency was caused by expression or whether it was a defect in HDR, levels of integration were quantified using digital droplet PCR. See e.g., Hung, K. L. et al, (2018) Molecular Therapy, 26:456-467, which is expressly incorporated by reference herein in its entirety).

As shown in FIG. 8E, the allele frequency of HDR events for the BFP templates were similar to the proportion observed when quantifying BFP+ cells (e.g. 14.5% alleles vs/11.2% BFP+; ˜ 1.34 fold difference on average). In contrast, the allele frequency of HDR events for both BTCE templates was significantly lower than expected (e.g. 8.6 percent alleles vs/2.79% GFP+; ˜ 3.45 fold difference on average). These data indicated that the BTCE genes were specifically not expressed efficiently at the CCR5 locus.

### Example 7—Quantification of Expressed Blinatumomab

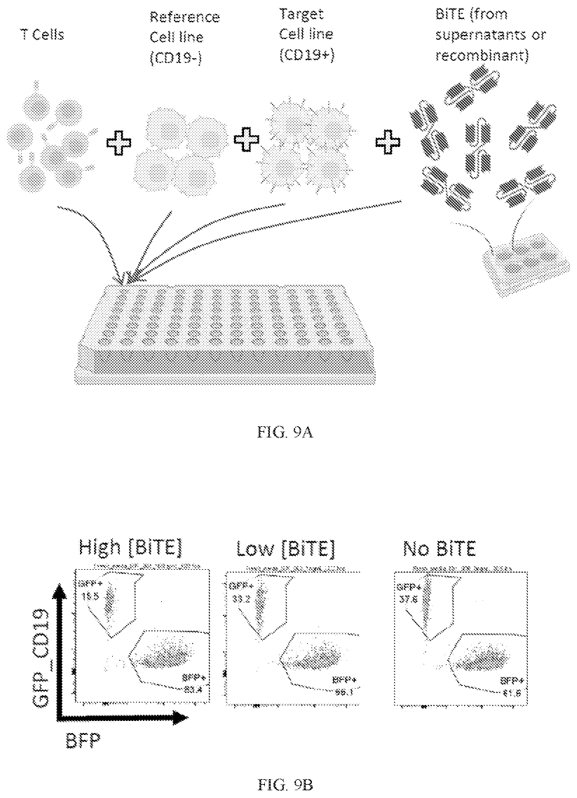

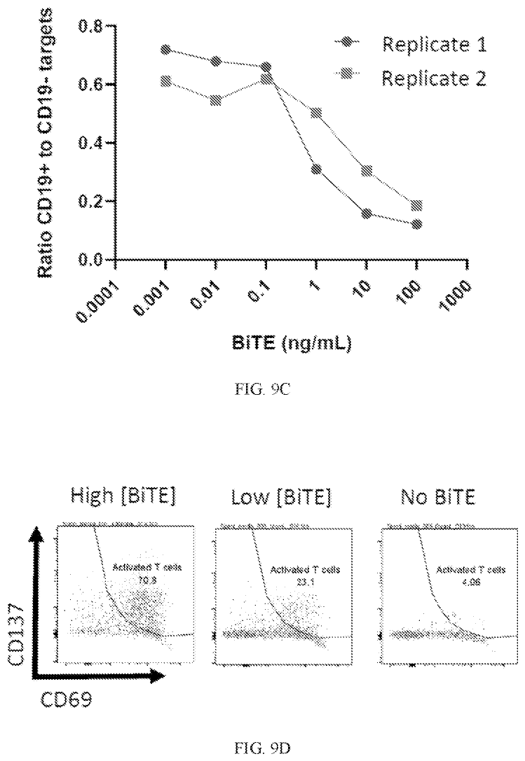

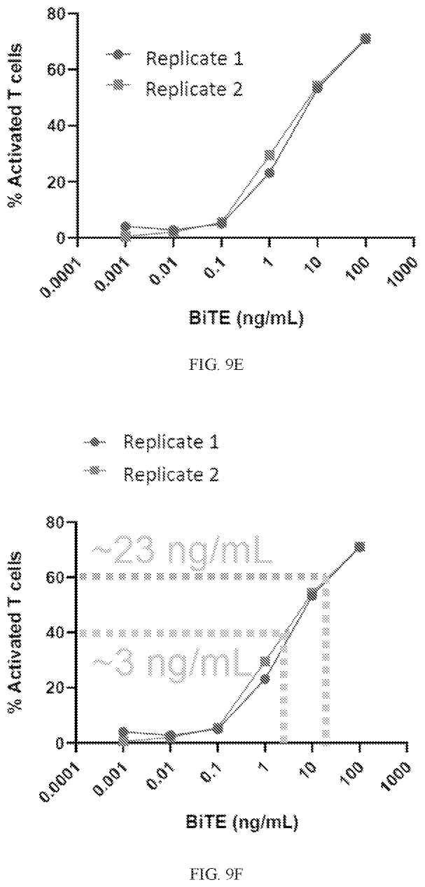

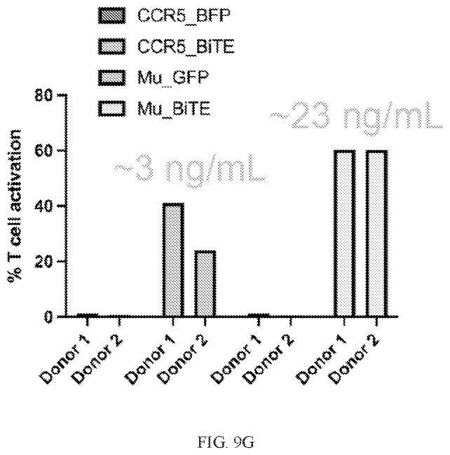

FIG. 9A is a schematic for a method used to quantify secreted blinatumomab. In this assay, BTCE or vehicle media was incubated with T cells, a reference cell line expressing BFP (K562; BFP+CD19-), but not CD19 and a target cell line that expresses GFP and CD19 (K562; GFP+CD19+). This mixture was incubated overnight. After incubation BTCE activity was quantified by measuring T cell activation (expression of CD69 and CD137) and T cell killing (changes in the ratio of target (GFP+) to reference (BFP+) cell.

A titration curve was also established using recombinant blinatumomab in which target cell killing (GFP versus BFP) was quantified following incubation of cells with various concentrations of blinatumomab. A FACS analysis was performed (FIG. 9B). As shown in FIG. 9C, increasing blinatumomab concentration caused a decrease in the percentages of GFP+ target cells in a reproducible manner.

T cell activation was quantified following incubation of cells with various concentrations of blinatumomab. A FACS analysis was performed (FIG. 9D). As shown in FIG. 9E, increasing blinatumomab concentration caused an increase in the percentages of CD3+ T cells that co-express CD137 and CD69 in a highly reproducible manner.

Supernatants were quantified using the T cell activation and target cell killing assays. As shown in FIG. 9F, the T cell activation elicited by B cell supernatants was plotted for each of the samples. The data was interpreted using a standard curve generated in a parallel study using recombinant blinatumomab. HDR templates integrated at the E-mu (Mu) locus resulted in substantially higher blinatumomab secretion than those integrated into the CCR5 locus (FIG. 9G).

### Example 8—Editing CCR5, IGHG1 and Eμ Loci

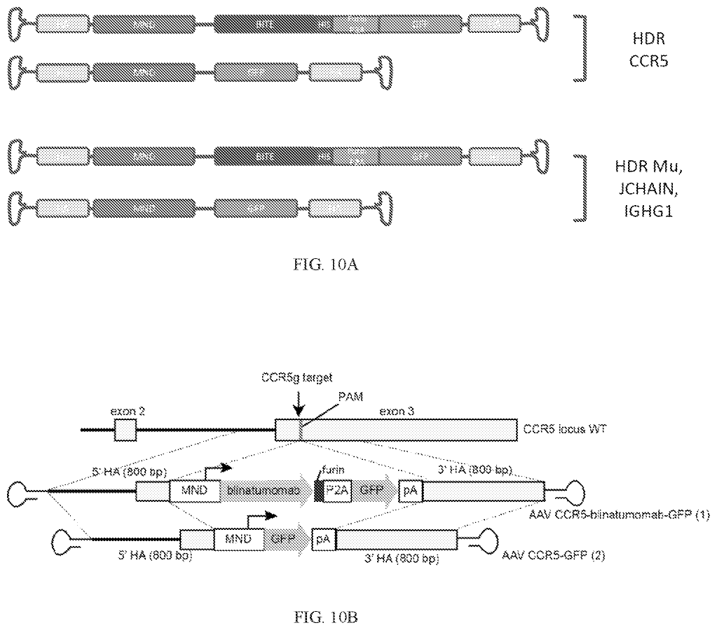

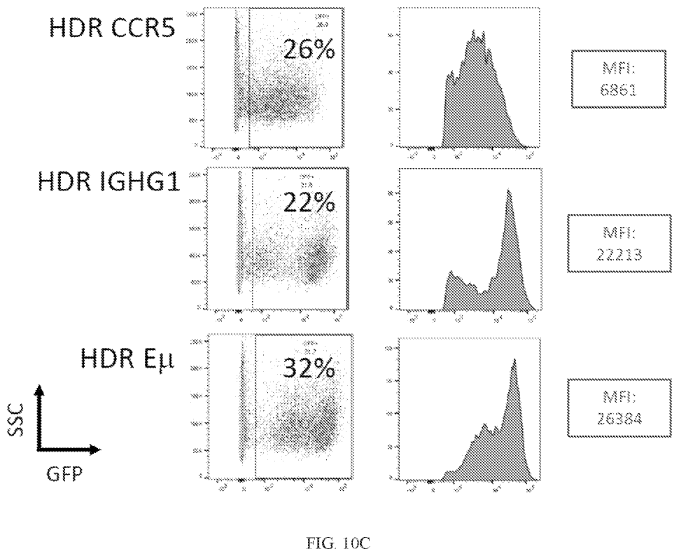

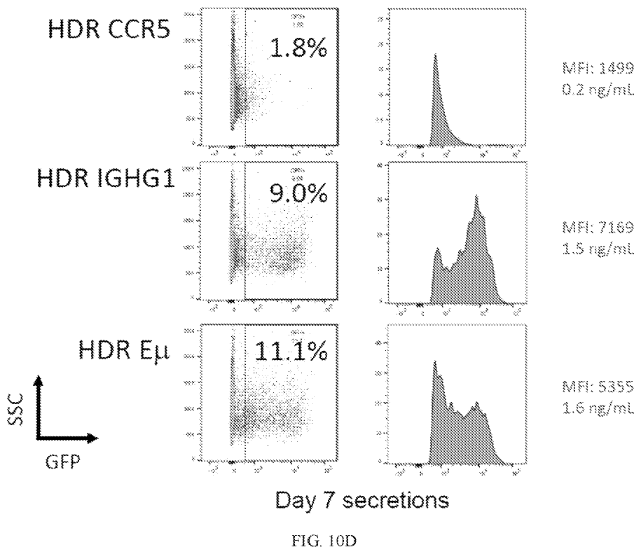

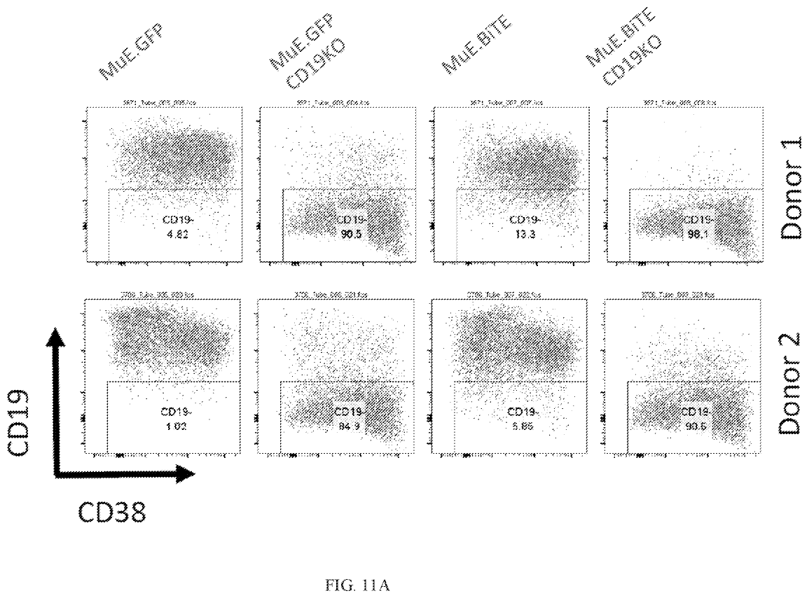

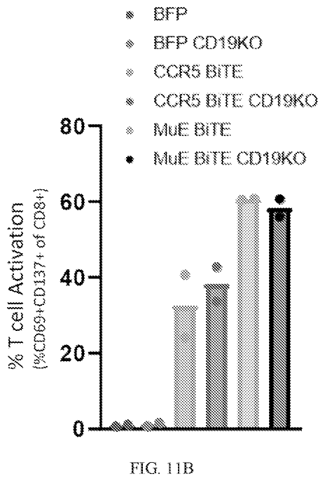

HDR templates were cloned into AAV vector for the expression of GFP or blinatumomab_2A_GFP at 4 different loci (FIG. 10A). For the CCR5 gene, an insertion into exon was targeted (FIG. 10B).

B cells were edited using methods described above with construct 2, containing arms of homology, and a GFP linked to an MND promoter. A FACS analysis was performed. As shown in FIG. 10C, HDR of the construct 2 expressing GFP into the indicated loci was measured. These data showed that the proportion of edited cells is similar at CCR5 and the two antibody loci (IGHG1 and E-mu). It was also demonstrated that there were increases in mean fluorescent intensity for GFP in cells edited at each antibody locus (see histograms and the quantification on the right portion of FIG. 10C). Also noted was that editing rates were similar at all loci. GFP MFI slightly increased at mAb loci (similar to prior JCHAIN results). Two peaks at Eμ.

B cells were edited using methods described above with construct 1, containing arms of homology, and a BTCE linked to an MND promoter, in cis with and a GFP linked to promoter. A FACS analysis was performed. As shown in FIG. 10D, HDR of the construct 1 expressing Blinatumomab and GFP into the indicated loci was measured. These data showed that the proportion of edited cells is approximately 5 times higher at the two antibody loci (IGHG1 and E-mu). It also demonstrated that there were marked increases in mean fluorescent intensity for GFP in cells edited at each antibody locus (see grey histograms and the quantification on the right of FIG. 10D). Also noted was that HDR for constructs encoding binders were less efficient than GFP alone. HDR ˜10-15%. Expression best at IGHG1 locus. Blinatumomab secretion similar at IGHG1 and Em. JCHAIN data (not shown) was substantially similar as that for CCR5. Using the T cell activation assay, the amount of blinatumomab produced by the B cells was quantified, and found to be ˜8× higher in cells edited at the antibody loci.

### Example 9—Effects of Inactivating CD19 in B Cells Expressing Blinatumomab

B cells were edited to express GFP and/or a BTCE. Flow cytometry was used to quantify CD19 and CD38 (a marker of plasma cells) in the engineered cell population. It was found that CD19 knockout did not eliminate surface expression of CD19 without marked effects of the differentiation of B cells into plasma cells (see CD38 stain in FIG. 11A). Supernatants were isolated and incubated with CD19+ K562, CD19− K562 and T cells as described above. To measure the activity of blinatumomab in the B cell supernatants, T cell activation in these co-culture was quantified (FIG. 11B). CD19 knockout did not have a marked effect on the production of blinatumomab by edited B cells.

CD19 inactivation in cells should prevent fratricide by of engineered B cells that co-express CD19.

### Example 10—In Vivo Activity of B Cells Expressing Blinatumomab

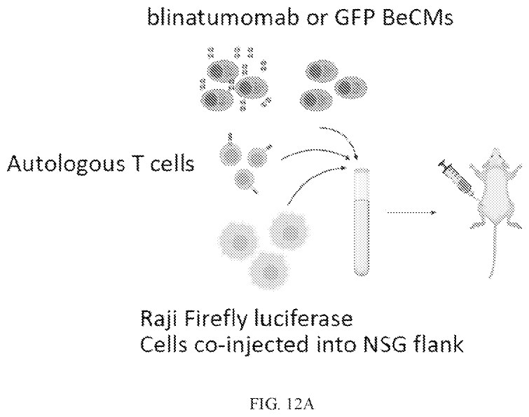

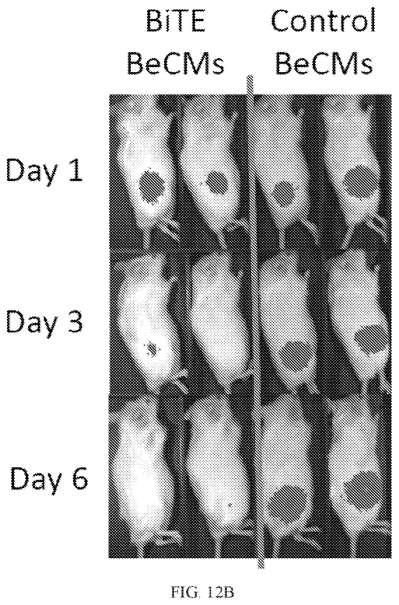

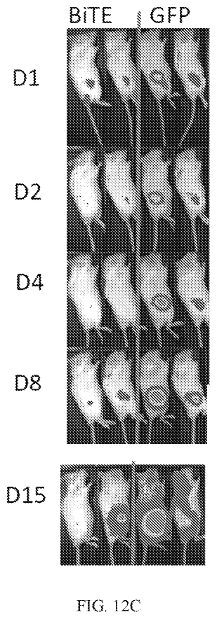

B cells were genetically modified to express blinatumomab (test) or GFP (control). Modified B cells were mixed with autologous T cells, and Raji cells (CD19+B cell lymphoma) which had been modified to express luciferase. The cell mixture was implanted into the flank of immunodeficient mice (NSG) (FIG. 12A). Following implantation, in vivo tumor growth was measured by measuring luminescence (blue circle in mice) with an IVIS instrument. In mice implanted with B cells producing blinatumomab, the CD19+ tumor cells were eliminated, thus demonstrating in vivo efficacy of B cell producing BTCEs (FIG. 12B).

In another study, humanized immunodeficient mice were generated by engrafting NSG mice with 1 million human CD34 cells at ˜12 weeks of age. The human immune cells were allowed to differentiate in the mice for 8 additional weeks, resulting in the differentiation of the CD34 cells into various human lymphocytes including monocytes, other myeloid cells, B cells and some T cells. It was predicted that T cells from the host could potentially interact with the BTCE and elicit a cytotoxic response against the Raji tumor cells.

B cells expressing either blinatumomab (test) or GFP (control) were mixed with Raji which expressed luciferase. The cell mixture was implanted into the humanized immunodeficient mice. Mice that had been engrafted with blinatumomab-secreting B cells had substantially less tumor growth relative to mice engrafted with B cells expressing GFP (FIG. 12C).

### Example 11—Engineered Plasma Cells Secrete Bispecific T Cell Engagers and Suppress Patient-Derived Leukemia In Vivo

Bispecific T cell Engagers (BTCEs) are an important tool for the management and treatment of human hematological malignancies. However, BTCEs are not used more generally due to their short serum half-life and on-target toxicity at non-tumor sites. Advances in genome-engineering have enabled the generation of human plasma cells that secrete large quantities of therapeutic proteins. These engineered cells are capable of long-term in vivo engraftment in humanized mouse models. As a next step towards clinical translation of engineered plasma cells (ePCs) towards cancer therapy, described herein are certain embodiments for the expression and secretion of BTCEs by human plasma cells. As disclosed herein, human plasma cells engineered to express either anti-CD19 (blinatumomab) or anti-CD33 (AMG 330) BTCEs were capable of mediating T cell activation and direct T cell killing of cancer cell lines and primary human cells in vitro. Furthermore, BTCE-ePCs elicited tumor eradication in vivo following local delivery adjacent to flank-implanted tumor cells. Finally, immunocompromised mice engrafted with T cells and ePCs secreting anti-CD19-BTCE prevented in vivo growth of CD19⁺ acute lymphoblastic leukemia patient derived xenografts. These findings support use of ePCs as a durable local delivery system of BTCEs for the treatment of leukemias, lymphomas, and other cancers.

### Human Primary B Cells Engineered by CRISPR Cas9 Homology Directed Repair Secrete Functional BTCEs

To integrate a BTCE gene expression cassette into B cells, AAV-based homology directed repair templates (HDR) were adapted that had been used for delivery of transgenes in B cells at the safe harbor gene CCR5¹⁹. CCR5-targeted HDR templates were designed for delivery of BFP or the anti-CD19 BTCE blinatumomab cis-linked with GFP. In a previously established B cell editing protocol¹⁹, primary human B cells were isolated from peripheral blood mononuclear cells (PBMCs) and expanded the cells in culture for 2 days using a cytokine cocktail that drives activation of primary B cells (expansionary media) and includes oligomerized CD40 ligand, CpG, interleukin-2 (IL2), IL10 and IL15. Next gene editing was initiated by transfecting the activated B cells with Cas9 ribonucleoprotein complexes (RNPs) containing guide RNAs targeting sequence within CCR5, and subsequently transduced with the aforementioned AAV vector. Prior to analysis, engineered B cells were cultured in expansionary media for five additional days (FIG. 13A, FIG. 17A). It was found that HDR integration rates were slightly lower with the vectors containing BTCEs when evaluated by digital droplet PCR (ddPCR; FIG. 13B, FIG. 17). However, despite similar integration rates the proportion of cells expressing the fluorescent reporter was substantially diminished in cells edited using the BTCE design, resulting in a significant drop in the ratio fluorescent reporter marking to integration rate (FIGS. 13C-13D).

It was hypothesized that BTCE transgene expression could be increased by targeting transgene integration to loci that are natively expressed in B cells or plasma cells. Therefore, three additional AAV-based repair template designs were built for delivery of transgene cassettes to the highly expressed B cell loci IGHG1, JCHAIN, and a region proximal to an heavy chain enhancer, 12u (repair arms and sgRNA were previously described in 12; overall schematic for all vectors, FIG. 13E). Although the same promoter was used at all loci (the viral promoter, MND27), variable increases were observed in GFP percentage and mean fluorescent intensity in B cells following delivery of the fluorescent reporter to the antibody-associated loci, relative to that at CCR5 (FIGS. 17C-17D). While integration of the BTCE was detected at all loci, significant increases were observed in the mean fluorescent intensity of the cis-linked GFP at the antibody loci relative to CCR5 (FIGS. 13F-13G).

To determine the functionality of the B cell-produced anti-CD19 BTCE, an in vitro killing assay was developed. Briefly, lentivirus was used to transduce CD19 and GFP stably into target K562 cells and BFP into reference K562 cells. CD19− BFP+ reference cells, CD19+GFP+ target cells and PBMC-derived CD8+ T cells were incubated with either recombinant anti-CD19 BTCE or with supernatants from engineered cells (FIG. 13H). After 48 hours, flow cytometry was used to quantify the degree of T cell activation (percent CD39+CD137+) and specific lysis (ratio of GFP to BFP K562 cells) of CD19+ target cells (FIG. 18A). Recombinant anti-CD19 BTCE elicited dose-dependent increases in T cell activation and CD19-specific lysis (FIG. 18B). Supernatants from B cells engineered to express anti-CD19 BTCE induced robust T cell activation and specific lysis of CD19+ cells, whereas supernatants from GFP-engineered B cells did not (FIGS. 131-13J). Using the T cell activation assay, standard curves were generated from the recombinant BTCE data and determined that the engineered B cells produced high concentrations of anti-CD19 BTCE in the supernatants (FIG. 13K). These findings indicated that primary human B cells can be engineered at various loci to robustly express functional anti-CD19 BTCE.

### BTCE-Engineered Plasma Cells Show Robust In Vitro Efficacy Against Common Leukemia Target Antigens

Whether ex vivo-differentiated human plasma cells could produce BTCEs that specifically target antigen-expressing cells within heterogeneous primary human cell populations was determined. Using a similar design to the anti-CD19 Eu-directed BTCE (aCD19) design, an additional repair template was built for delivery of an anti-CD33 BTCE (aCD33; FIG. 14A). Each BTCE, and the GFP control was introduced into B cells using HDR and differentiated the cells into ePCs as previously described (FIG. 17A)¹⁹. Following editing and differentiation, detectable transgene expression was observed with all vectors and donors (FIGS. 14B-14C). Although donor-dependent differences were observed in expression of the plasma cell differentiation markers CD38 and CD138 (FIG. 19B), introduction of the BTCEs did not impact differentiation into plasma cells (defined as CD38++ CD138+; FIGS. 19A-19B). Collectively, these data demonstrated that plasma cells can be engineered to express BTCEs.

The functionality of BTCE secreted by the ePCs was assessed using a PBMC killing assay. In this assay, effector CD8+ T cells were co-cultured with autologous. PBMCs that contained B and myeloid cell subpopulations that express CD19 and CD33 respectively. To elicit killing, either recombinant BTCE or supernatants derived from ePCs were added (FIG. 13D). 48 hours after adding BTCE, target cell numbers and phenotype were quantified using flow cytometry (FIG. 19D). As expected, it was found that recombinant αCD19 BTCE elicited a dose-dependent decrease in IgM+B cells, whereas recombinant αCD33 BTCE elicited a dose-dependent decrease in CD14+ CD33+ monocytes (FIG. 19E). It was found that supernatants from both αCD19- and αCD33-ePCs elicited higher T cell activation relative to that from GFP-ePCs (FIG. 14F). Furthermore, supernatants from αCD19-ePCs specifically killed target IgM+B cells (FIG. 14G), while supernatants from αCD33-ePCs specifically killed target CD33+ CD14+ monocytes (FIG. 14H). Likewise flow cytometry was used to quantify T cell activation (% CD69+, CD137+ of CD8+ cells) (FIG. 14H); the frequency of NAML-6 (CD19+) (FIG. 14I); and MOLM-14 (CD33+) (FIG. 14J) in the leukemia killing assay. This data indicated that BTCE producing ePCs can elicit specific T cell targeting of cells expressing leukemia targets in vitro.

### Plasma Cells Engineered to Remove Auto Expressed Antigens Prevents Self-Targeting and Boosts BTCE Expression

CAR T cells engineered to recognize T-cell antigens can kill other CAR T cells within the same cell product, resulting in diminished anticancer activity^{28,29}. It was hypothesized that upon T cell encounter, αCD19-ePCs could similarly elicit self-targeting due to their expression of CD19 (FIG. 20). To evaluate the degree of self-targeting, either GFP-ePCs or αCD19-ePCs were incubated with autologous T cells (FIG. 15A). After 24 hrs, the proportion of αCD19-ePCs were quantified by flow cytometry. It was found that starting the assay with higher numbers of T cells resulted in decreased percentages of αCD19-, but not GFP-ePCs (FIG. 15B, FIG. 21), which implied that CD19-specific self-targeting could impact BTCE-secreting ePCs.

It was investigated whether elimination of CD19 would prevent BTCE-elicited self-targeting. To knockout CD19, RNPs targeting CD19 were co-delivered with the μ-directed αCD19 BTCE editing reagents. The addition of the CD19-targeting RNPs resulted in >85% reduction in the proportion of CD19+ plasma cells (FIGS. 15C-15D). CD19 knockout did not overtly impact differentiation of edited B cells into plasmablasts or plasma cells in vitro (FIG. 22). Upon challenging these CD19 knockout CD19-ePCs with T cells, no differences were observed in GFP percentage with increased starting T cell numbers (FIGS. 15E-15F). These data suggested that CD19 knockout protected αCD19-ePCs from self-targeted death.

An additional challenge with self-expression of CD19 is that the αCD19 BTCE produced from engineered cells are likely to bind to CD19 and limit the quantity that is released by the cells. Therefore, it was investigated whether CD19 knockout would lead to higher detection of αCD19 BTCE in supernatants. To assess free BTCE in the context of CD19 knockout, supernatants from BTCE ePCs were evaluated co-engineered with or without CD19 RNPs in the K562 killing assay (FIG. 13E). It was found that supernatants from CD19KO «CD19-ePCs resulted in higher T cell activation, higher specific lysis, and higher αCD19 BTCE concentrations when compared to CD19WT αCD19-ePCs (FIGS. 15G-151). Collectively these indicated that CD19 knockout prevented self-targeting by T cells and boosts &CD19 BTCE levels.

Plasma Cells Engineered to Express BTCES have Anti-Tumor Efficacy In Vivo

To test whether BTCE-secreting ePCss maintained function in vivo, a flank model of B cell leukemia was used. Immunocompromised NOD.Cg-Prkdcscid Il2rgtm1 Wj1/SzJ-c (NSG) mice were subcutaneously injected with CD19+ ffLuciferase Raji cells, along with autologous T cells and CD19KO ePCs expressing either GFP or αCD19 BTCE (FIG. 16A. It was found that the luciferase-expressing Raji cells engrafted similarly in all groups (day 1 time point, FIGS. 16C-16D). However, at later time points, tumor burden decreased in mice that received αCD19-ePCs relative to mice that received GFP-ePCs (FIGS. 16B-16C). In the BTCE group, reductions in tumor size were below background luminescence levels in >50% of the mice within 5 days (FIGS. 16B-16C). These findings demonstrated that αCD19-ePCs can promote robust local anti-tumor responses in vivo.

Blinatumomab is prescribed to B-ALL patients with minimal residual disease, as a bridge to transplant, following chemotherapy³⁰⁻³⁵. To mimic conditions observed in B-ALL patients after lymphoablative therapy, a model was developed where BTCE-ePCs were delivered prior to tumor and to T cells. NSG Mice were intravenously injected with CD19KO GFP or αCD19-ePCs. The following day, luciferase expressing cells were intravenously injected from an aggressive patient-derived xenograft model of PH-like B-ALL (NL482B; IL7R gain-of-function, SH2B3 deletion)^{36,37}. Finally, effector T cells that were syngeneic with the ePCs were injected retro-orbitally one and three days after the B-ALL engraftment (FIG. 16D). Tumor growth was monitored until the luminescence signal became saturated in GFP control mice (FIGS. 16E-16F). Mice that received αCD19-ePCs showed near complete control of the tumor, and significant decreases in tumor burden relative to the control groups (FIGS. 16F-16G). It was also observed a trend towards higher T cell frequencies in the peripheral blood of the αCD19-ePC treated group compared to GFP control group 15 days post tumor engraftment, which is consistent with BTCE-driven expansion of T cells in vivo (FIG. 23B). There was a drastic decrease in CD19+ cells in the spleens and bone marrow in the αCD19-ePC treated group compared to control groups upon sacrifice at 34 days post-tumor transfer (FIG. 23C). BTCE-ePCs were capable of preventing dissemination of a patient-derived leukemia in a model that functionally imitates the clinical use of blinatumomab as a bridge therapy in B-ALL patients.

ePCs are emerging as an attractive modality for the delivery of therapeutic proteins; here, the use of ePCs to produce BTCEs is disclosed. Using HDR high expression of multiple BTCEs was achieved at multiple active loci in primary human B cells. It was demonstrated that B cells engineered to express BTCEs could be differentiated into plasma cells that mediated killing of primary human cells expressing CD19 or CD33. Knockout of the target antigen, CD19, boosted αCD19 BTCE secretion by ePCs and prevented self-targeting. It was also shown that BTCE ePCs were capable of directing a robust T cell dependent anti-tumor response against an engrafted lymphoma cell line and a disseminated patient-derived leukemia xenograft model that mirrors BTCE treatment in B-ALL patients.

Standard of care for patients with B-ALL is an initial course of lymphoablative chemotherapy, which is followed by consolidation and transplant in the case of individuals at high risk for refractory disease³⁸. Lymphoablative therapy depletes circulating B cells and is likely to introduce further genetic abnormalities that, in combination with inherited risk, could accelerate transformation of the remaining B cells^{39,40}, or those in an ePC product. Another safety concern for B-ALL recipients is that following lypmhoablative therapy, EBV-reactive T cells are likely to be depleted, which could increase the risk for EBV-driven lymphoproliferative disease⁴¹, either from the remaining recipient B cells or from an ePC product. For these reasons, a useful αCD19-ePC product for B-ALL can be sourced from an EBV-, allogeneic donor. In addition to alleviating the aforementioned safety concerns, use of an allogeneic donor has two significant benefits. First, donors could be prioritized for use in multiple recipients based on manufacturability, potentially overcoming manufacturing concerns observed in other cell therapy modalities in heavily pretreated B-ALL⁴². Second, allogeneic BTCE-cell products, as a bridge to hematopoietic stem cell transplant, may have the added safety benefit of host clearance by allogeneic T cells^{43,44} derived from the bone marrow transplant.

A potential barrier for use of ePCs for treatment of leukemia, lymphoma or myeloma is that ePCs¹⁸ retain expression of surface markers targeted by many clinically available biologics (e.g. CD19, CD20, CD38, and BCMA), which could result in self-targeting of the ePC. In fact, data disclosed herein show that CD19+, αCD19-ePCs were eliminated when cultured in the presence of T cells. Because CD19 is not critical for plasma cell function^{48,49}, and is downregulated in long-lived PCS^{48,50,51}, an a engineering strategy disclosed herein for concurrent CD19 knockout and expression of αCD19 BTCEs is unlikely to negatively affect the function or longevity of the resulting ePC. However, a useful ePC product would likely include engineering with a single edit for expression of the αCD19 BTCE at the CD19 locus to avoid oncogenic Eμ translocations⁵²⁻⁵⁵. ePCs could be engineered for BTCE- or monoclonal antibody-based targeting of several B cell expressed tumor targets. Like CD19, knockout or depletion of the lymphoma and myeloma targets MS4A1 (also known as CD20)⁵⁶⁻⁵⁹, and CD38^{60,61} in plasma cells does not acutely impact their ability to provide long lasting durable antibody titers, a corollary of their longevity and secretory capacity. In contrast, knockout of TNFRSF17 (also known as BCMA) in mice decreases plasma cell survival and eliminates the antibody response⁶²; hence, knockout of TNFRSF17 would likely hamper the longevity and/or function of an ePC product. Therefore, engineering ePCs expressing biologics that target receptors expressed on B cells is likely feasible, and could enable ePC generation for BTCEs or monoclonal antibodies in use for chronic lymphocytic lymphoma (CD20; glofitamab⁶³), non-Hodgkin's lymphomas (CD20; rituximab⁶⁴ odronextamab⁶⁵, mosunetuzumab⁶⁶) and multiple myeloma (CD38; daratumomab⁶⁷, Bi38⁶⁸).

A primary benefit of using ePCs for delivery of biologics is mitigation of on-target drug effects that occur in healthy tissues due to systemic high dose delivery of the biologic. While ePCs can localize the bone marrow¹⁸ and functionally eliminate B-ALL cells that also localize to the bone marrow²⁶, ePCs could be used for local delivery in other types of liquid cancers and possibly even solid tumors. In addition to B-ALL, several forms of B cell lymphoma^{69,70} and myeloid leukemia⁷¹ have reservoirs in the bone marrow and other lymphoid tissues (spleen, lymph nodes) where ePCs reside in vivo^{18,72}. The tissue localization of plasma cells could be particularly useful for the local delivery of BTCEs for acute myeloid leukemia that have well-described on-target effects on healthy cells such as CD33 and CD123 targeting agents⁷³⁻⁷⁵. Plasma cells are also known to normally reside in additional tissues, including lung and gut epithelia⁷⁶ and even solid tumors⁷⁷⁻⁸⁰. In fact, tracking of donor-specific microbiota-specific IgA generated during infancy in solid organ transplant recipients shows that PCs producing antibodies in the gut can live for decades^{81,82}, which implies that ePCs delivered to solid tumors in mucosal sites have the potential for similar longevity. The first BTCE-like biologic approved for a solid tumor, tebentafusp, has a half-life of only 7.5 hours and requires weekly infusions to treat highly vascularized uveal cancer⁸³ provides evidence that BTCEs can be an effective strategy for solid tumors. However, systemic delivery of BTCEs targeting key solid tumor targets such as EGFR can lead to adverse on-target off-tumor toxicity⁸⁴. An active area of future research is to evaluate the localization and on-target off-tumor effects of ePCs secreting solid tumor biologics following direct delivery to tumor sites.

Therapeutic protein biologics were the second most approved drugs from 2009 to 201785 and many suffer from suboptimal half-lives. Because of their extended in vivo longevity¹⁸, ePCs are likely to confer extended drug availability in clinical applications where short half-life limits use/adoption. Biologics used in chronic treatment are especially challenging due to life-long frequent dosing requirements (sometimes daily), and include those used for enzyme replacement (agalsidase beta; 56 to 76 minutes⁸⁶, factor IX; 18 to 40 hours⁸⁷ laronidase; 1.5 to 3.6 hours⁸⁸), chronic autoimmune disorders (infliximab; 9.5 days⁸⁹ etanercept; 80 hours⁹⁰), diabetes (liraglutide; 13 hours⁹¹) and human immunodeficiency virus (enfuvirtide 3.4 hours⁹²). The phenotype of BTCE-engineered cells described herein (CD38++ CD138+) resembles the phenotype of long-lived plasma cells from human bone marrow⁵⁰ and have previously been shown to persist in humanized mice for over a year¹⁸. ePCs ability to persist long term and produce robust levels of exogenous protein could be a key to unlocking the therapeutic potential of many biologics and therapeutic peptides that lack efficacy due to poor pharmacokinetics.

### Experimental Methods

### AAV6 HDR CRISPR Cas9 Engineering

CRISPR RNAs (crRNAs) targeting the CCR5, JCHAIN, IgG1, Eμ¹², and CD19 were identified using the (http://portals.broadinstitute.org/gpp/public/analysis-tools/sgrna-design), and are listed in the following TABLE 9.

TABLE 9

| Guide target | SEQ ID NO | Sequence |
|---|---|---|
| CCR5 | SEQ ID NO: 22 | CAATGTGTCAACTCTTGACA |
| JCHAIN | SEQ ID NO: 23 | AAGAACCATTTGCTTTTCTG |
| IgHG1 | SEQ ID NO: 02 | TGAGTTTTGTCACAAGATTT |
| Eμ | SEQ ID NO: 01 | GTCTCAGGAGCGGTGTCTGT |
| CD19 | SEQ ID NO: 24 | CCTCGGGCCTGACTTCCATG |

crRNAs were synthesized (IDT) containing phosphorothioate linkages and 2′O-methyl modifications. crRNA and trans-activating crRNA (tracrRNA; IDT) single guide hybrids were mixed with 3 uM Cas9 nuclease (Berkeley Labs) at a 1.2:1 ratio and delivered to cells by Lonza 3D (CA-137) or Maxcyte GTX (B cell 3) electroporation. After electroporation, cells were transferred into the activation medium (1.5 million cells/mL) in the presence of AAV6 vectors carrying homologous DNA repair templates (20% AAV by volume). The medium was changed 24 hours following AAV6 administration. AAV vectors were produced as previously described 19.

### In Vitro Killing Assays (K562, PBMC, Self-Killing)

For the K562 killing assay, K562 cells were obtained from ATCC and lentiviraly transduced to express either CD19 linked in cis to GFP (target) via self-cleaving P2A or BCMA linked in cis to BFP (reference) and purified by flow assisted sorting. 5E3 Target, 5E3 reference cells, 5E4 CD8+ T cells were incubated with either various dilutions of supernatants from genome-engineered cells or media containing various concentrations of recombinant BTCE (Invivogen, bimab-hcd19cd3) for 48 hours at (FIGS. 13F, 13H, 15G-15I), For the PBMC killing assay, 2E5 PBMCs and 4E4 autologous CD8+ T cells were incubated with either supernatants from genome-engineered cells or media containing recombinant BTCE (Invivogen, bimab-hcd19cd3, AMG330) for 48 hours at (FIGS. 14D-14G). For the self-killing assay, 2E5 genome-engineered B cells were incubated with autologous T cells at various effector to target ratios and cultured for 24 hours (FIGS. 15B, 15E, 15F). Each assay was performed in 200 μL in duplicate in 96 wells with RPMI-1640 supplemented with 10% FBS as the base media at 37° C. and 5% CO₂. At the end of each assay, duplicate wells were pooled, washed with PBS, stained and analyzed by flow cytometry.

### NSG Mouse Models

NOD.Cg-Prkdc^{scid} Il2rg^{tm1Wjl}/SzJ-c (NSG) mice were purchased from Jackson Laboratory. For the subcutaneous flank model (FIGS. 16A-16C), 2.5E5 million ePCs, 5E4 autologous T cells, and 2.5E4 Raji.Luciferase cells were delivered subcutaneously to the right flank. For the disseminated NL482 PDX model (FIGS. 16D-16G), 2.5E6-15E6 GFP or BTCE-ePCs were injected intravenously. The following day mice received 1E5 NL482.ffLuc ALL cells intravenously. The following day and three days later, mice received 1 or 10E5 T cells retro-orbitally. Tumor engraftment was monitored by bioluminescence imaging using IVIS Lumina S5 (Perkin Elmer) following subcutaneous injection of luciferin (75-150 mg/kg). Peripheral blood was collected via submandibular bleed and processed for serum (BD 2290057). Mice were euthanized and then bone marrow and spleens were harvested, processed to lyse RBCs (ACK lysis) and then phenotyped by flow cytometry (FIGS. 23A-23C). Animals were housed at ambient temperature and humidity.

### Statistical Analysis and Data Availability

Statistical analyses were performed using Prism 7 (GraphPad, San Diego, CA). It was assumed all data followed a normal distribution.

### Cell Lines

K562, Raji, were obtained from the ATCC and cultured in RPMI 1640 supplemented with 10% fetal bovine serum. K562 were retroviral transduced with VSV-g pseudo-typed virus containing CD19 in cis to GFP or BCMA (NP_001183) in cis to BFP and subsequently purified using fluorescence activated cell sorting (FACS) to generate target K562 CD19⁺ GFP⁺ and reference K562 BCMA⁺ BFP⁺ cell lines. Raji were lentivirally transduced with firefly luciferase (AB261984.1) and enhanced green fluorescent protein (HM640279.1) and FACS-purified.

### Flow Cytometry

Cells were stained with appropriate antibodies. Flow cytometric analysis was performed on an LSR II flow cytometer (BD Biosciences) and events were analyzed using FlowJo software (Tree Star).

### B Cell Culturing and Plasma Cells Differentiation

B cells were isolated from healthy human donors' PBMCs using the EasySep Human B cell isolation kit (Stem Cell Technologies). >95% purity was obtained for B cells defined by CD3 negativity and CD19 positivity. Isolated B cells were cultured in Iscove's modified Dulbecco's medium (Gibco), supplemented with 2-mercaptoethanol (55 μM) and 10% FBS. Cells were cultured for 7 days (activation and expansion) in medium containing 100 ng/mL megaCD40L (Enzo Life Science), 1 μg/ml CpG ODN2006 (Invitrogen), 50 ng/ml IL-2, 50 ng/ml IL-10 (Peprotech), and 10 ng/ml IL-15 (Miltenyi), for 3 days (differentiation) in medium containing 50 ng/ml IL-6 (Peprotech) 50 ng/ml IL-2, 50 ng/ml IL-10, and 10 ng/ml IL-15 and 2 days (plasma cell terminal differentiation) in medium containing 50 ng/ml IL-6, 10 ng/ml IL-15, and 15 ng/ml interferon-α2B (Sigma-Aldrich). Cell concentrations were kept between 5-15E5 live cells per ml. Cells for in vivo experiments were purified via CD3 bead depletion column (Miltenyi) prior to injection.

### REFERENCES

1. Mullard, A. Nat. Rev. Drug Discov. 16, 669 (2017).
2. Waldman, A. D., et al. Nat. Rev. Immunol. 20, 651-668 (2020).
3. Siegel, R. L., et al. C A Cancer J. Clin. 71, 7-33 (2021).
4. Kantarjian, H. et al. N. Engl. J. Med. 376, 836-847 (2017).
5. Przepiorka, D. et al. Clin. Cancer Res. 21, 4035-4039 (2015).
6. Halford, Z., et al. Ann. Pharmacother. 55, 1236-1253 (2021).
7. Zhu, M. et al. Clin. Pharmacokinet. 55, 1271-1288 (2016).
8. Toksvang, L. N., et al. Leukemia 36, 1749-1758 (2022).
9. Apostolidou, E. et al. Lymphoma Myeloma Leuk. 21, e895-e902 (2021).
10. Strohl, W. R. BioDrugs 29, 215-239 (2015).
11. Subklewe, M. et al. Blood 134, 833-833 (2019).
12. Moffett, H. F. et al. Sci Immunol 4, (2019).
13. Huang, D. et al. Vaccine Elicitation of HIV Broadly Neutralizing Antibodies from Engineered B cells.
14. Voss, J. E. et al. eLife vol. 8 Preprint at (2019).
15. Hartweger, H. et al. J. Exp. Med. 216, 1301-1310 (2019).
16. Nahmad, A. D. et al. Nat. Commun. 11, 5851 (2020).
17. Luo, B. et al. Cell Death Dis. 11, 973 (2020).
18. Cheng, R. Y.-H. et al. Nat. Commun. 13, 6110 (2022).
19. Hung, K. L. et al. Mol. Ther. 26, 456-467 (2018).
20. Manz, R. A., et al. Nature 388, 133-134 (1997).
21. Amanna, I. J., et al. N. Engl. J. Med. 357, 1903-1915 (2007).
22. Eyer, K. et al. Nat. Biotechnol. 35, 977-982 (2017).
23. Radbruch, A. et al. Nat. Rev. Immunol. 6, 741-750 (2006).
24. Marchand, T. & Pinho, S. Front. Immunol. 12, 775128 (2021).
25. Benet, Z., et al. Cell Rep. 34, 108733 (2021).
26. Tasian, S. K., et al. Biomedicines 6, (2018).
27. Robbins, P. B. et al. J. Virol. 71, 9466-9474 (1997).
28. Fleischer, L. C., et al. J. Hematol. Oncol. 12, 141 (2019).
29. Gower, M. & Tikhonova, A. N. Blood vol. 140 3-4 (2022).
30. Keating, A. K. et al. Blood Adv 3, 1926-1929 (2019)
31. Pawinska-Wasikowska, K. et al. Cancers 14, (2022)
32. Bargou, R. et al. Science 321, 974-977 (2008).
33. Mølhøj, M. et al. Mol. Immunol. 44, 1935-1943 (2007).
34. Dreier, T. et al. Int. J. Cancer 100, 690-697 (2002).
35. Dreier, T. et al. J. Immunol. 170, 4397-4402 (2003).
36. Bride, K. L. et al. Haematologica 107, 1746-1757 (2022).
37. Maude, S. L. et al. Blood 120, 3510-3518 (2012).
38. Künz, T. et al. Cancers 14, (2022)
39. Wiseman, D. H. Biol. Blood Marrow Transplant. 17, 771-789 (2011).
40. Ferrara, F. & Picardi, A. Cancers 12, (2019).
41. Fujimoto, A. & Suzuki, R. Cancers 12, (2020).
42. Roddie, C., et al. Cytotherapy 21, 327-340 (2019).
43. Jiang, H., et al. Biomark Res 8, 66 (2020).
44. Cao, X.-Y. et al. Blood 140, 4802-4803 (2022).
45. Cooper, M. L. et al. Leukemia 32, 1970-1983 (2018).
46. Mamonkin, M., et al. Blood 126, 983-992 (2015).
47. Gomes-Silva, D. et al. Blood 130, 285-296 (2017).
48. Mei, H. E. et al. Blood 125, 1739-1748 (2015).
49. Bhoj, V. G. et al. Blood 128, 360-370 (2016).
50. Halliley, J. L. et al. Immunity 43, 132-145 (2015).
51. Arumugakani, G. et al. J. Immunol. 198, 4618-4628 (2017).
52. Yan, Y., et al. A. Genes Chromosomes Cancer 46, 950-959 (2007).
53. Gostissa, M. et al. Nature 462, 803-807 (2009).
54. Park, S. S. et al. Cancer Res. 65, 1306-1315 (2005).
55. Hecht, J. L. & Aster, J. C. J. Clin. Oncol. 18, 3707-3721 (2000).
56. Hammarlund, E. et al. Nat. Commun. 8, 1781 (2017).
57. Kuijpers, T. W. et al. J. Clin. Invest. 120, 214-222 (2010).
58. Kozlova, V. et al. PLOS One 15, e0229170 (2020).
59. Langley, W. A. et al. J. Virol. 96, e0002622 (2022).
60. Hotchandani, N., et al. Am. J. Clin. Pathol. 146, (2016).
61. Postigo, J. et al. PLOS One 7, e33534 (2012).
62. O'Connor, B. P. et al. J. Exp. Med. 199, 91-98 (2004).
63. Papazoglou, D. et al. HemaSphere 6, 42 (2022)
64. Bannerji, R. et al. Lancet Haematol 9, e327-e339 (2022).
65. Dotan, E., et al. P T 35, 148-157 (2010).
66. Budde, L. E. et al. J. Clin. Oncol. 40, 481-491 (2022).
67. Abdallah, N. & Kumar, S. K. Ther. Adv. Hematol. 10, 2040620719894871 (2019).
68. Mei, H. et al. J. Hematol. Oncol. 14, 161 (2021).
69. Macintyre, E. A. et al. Blood 86, 4691-4698 (1995).
70. Martinez-Climent, J. A., et al. Haematologica 95, 293-302 (2010).
71. Lane, S. W., et al. Blood 114, 1150-1157 (2009)
72. Hill T F, et al. Molecular Therapy, 2022
73. Kenderian, S. S. et al. Leukemia 29, 1637-1647 (2015).
74. Guy, D. G. & Uy, G. L. Curr. Hematol. Malig. Rep. 13, 417-425 (2018).
75. Lamble, A. J. et al. J. Clin. Oncol. 40, 252-261 (2022).
76. Kiyono, H. & Fukuyama, S. Nat. Rev. Immunol. 4, 699-710 (2004).
77. Fristedt, R. et al. J. Gastrointest. Oncol. 7, 848-859 (2016).
78. Wouters, M. C. A. & Nelson, B. H. Clin. Cancer Res. 24, 6125-6135 (2018).
79. Yeong, J. et al. Front. Immunol. 9, 1209 (2018).
80. Hao, D. et al. Cancer Discov. 12, 2626-2645 (2022)
81. Jahnsen, F. L., et al. Trends Immunol. 39, 196-208 (2018).
82. Landsverk, O. J. B. et al. J. Exp. Med. 214, 309-317 (2017).
83. Hua, G., et al. J Adv Pract Oncol 13, 717-723 (2022),
84. Rosenthal, M. A. et al. Neuro. Oncol. 21, vi283 (2019)
85. Batta, A., et al. J Family Med Prim Care 9, 105-114 (2020).
86. Clarke, J. T. R., et al. Genet. Med. 9, 504-509 (2007).
87. Chhabra, A. et al. Blood Coagul. Fibrinolysis 31, 186-192 (2020).
88. Jameson, E., et al. T. Cochrane Database Syst. Rev. 6, CD009354 (2019).
89. Cornillie, F. et al. Aliment. Pharmacol. Ther. 15, 463-473 (2001)
90. Nestorov, I. et al. Br. J. Clin. Pharmacol. 62, 435-445 (2006).
91. Sisson, E. M. Pharmacotherapy 31, 896-911 (2011).
92. Cheng, S. et al. Eur. J. Med. Chem. 121, 232-237 (2016).

The term “comprising” as used herein is synonymous with “including,” “containing,” or “characterized by,” and is inclusive or open-ended and does not exclude additional, unrecited elements or method steps.

The above description discloses several methods and materials of the present invention. This invention is susceptible to modifications in the methods and materials, as well as alterations in the fabrication methods and equipment. Such modifications will become apparent to those skilled in the art from a consideration of this disclosure or practice of the invention disclosed herein. Consequently, it is not intended that this invention be limited to the specific embodiments disclosed herein, but that it cover all modifications and alternatives coming within the true scope and spirit of the invention.

All references cited herein, including but not limited to published and unpublished applications, patents, and literature references, are incorporated herein by reference in their entirety and are hereby made a part of this specification. To the extent publications and patents or patent applications incorporated by reference contradict the disclosure contained in the specification, the specification is intended to supersede and/or take precedence over any such contradictory material.

1.-70. (canceled)

71. A system for modifying a cell to express a bispecific T-cell engager (BTCE), comprising:
(a) a nuclease or nucleic acid encoding the nuclease; and
(b) a first polynucleotide encoding a homology direct repair (HDR) template comprising an expression cassette, wherein the expression cassette encodes the BTCE.

72. The system of claim 71, further comprising the cell, wherein the cell is a B cell or a B cell precursor.

73. The system of claim 71, wherein the nuclease is a Cas nuclease, and the system further comprises a second polynucleotide encoding a guide RNA (gRNA).

74. The system of claim 73, wherein the second polynucleotide comprises the nucleotide sequence set forth in any one of SEQ ID NOs: 01-27.

75. The system of claim 73, wherein the gRNA targets a locus selected from a CCR5 gene, a JCHAIN gene, an IGHM locus, a CD19 gene, or an IGHG1 gene.

76. The system of claim 71, wherein the BTCE comprises a first polypeptide which binds to a T-cell antigen, and a second polypeptide which binds to a tumor antigen.

77. The system of claim 76, wherein the T-cell antigen further comprises CD3; and the tumor antigen is selected from CD33, CD19, CD326 (EpCAM), neuron-glial antigen 2 (NG2), HER2, epidermal growth factor receptor (EGFR), CD66e, ephrin type-A receptor 2 (EphA2), CD21, FLT3, gp100, PDL1, or CD22.

78. The system of claim 71, wherein the BTCE comprises AMG 330, blinatumomab, solitomab, or tebentafusp.

79. The system of claim 71, wherein the expression cassette further comprises a promoter.

80. The system of claim 79, wherein the promoter is selected from an MND promoter, an IgVH promoter, an EF-1α promoter, or an IgHG1 promoter.

81. The system of claim 71, wherein the expression cassette further comprises one or more of:
(i) an enhancer selected from an Eμ enhancer or an SLC3A2 enhancer;
(ii) a polynucleotide encoding a signal sequence;
(iii) a polynucleotide comprising a 5′ UTR from an IGHV gene or an IGHG1 gene;
(iv) a polynucleotide comprising a 3′ UTR from an IGHV gene or an IGHG1 gene; or
(v) a ubiquitous chromatin-opening element (UCOE).

82. The system of claim 71, wherein the cell lacks expression of an endogenous protein to which the BTCE specifically binds.

83. A method for preparing a cell to express a bispecific T-cell engager (BTCE), comprising:
(a) obtaining the system of claim 71 and a cell; and
(b) introducing into the cell the nuclease or nucleic acid encoding the nuclease and the first polynucleotide to obtain a modified cell.

84. A cell prepared by the method of claim 83.

85. A pharmaceutical composition comprising the cell of claim 84.

86. A method for treating, ameliorating or inhibiting a disorder in a subject, comprising administering to the subject the cell of claim 84.

87. The method of claim 86, wherein the cell is administered in no more than a single dose.

88. The method of claim 86, wherein the disorder comprises a cancer.

89. The method of claim 88, wherein the cancer is selected from a solid tumor or a leukemia.

90. The method of claim 89, wherein the cancer is selected from acute lymphoblastic leukemia (ALL), acute myeloid leukemia (AML), breast cancer, stomach cancer, melanoma, colon cancer, colorectal cancer, head and neck cancer, gastric cancer, prostate cancer, ovarian cancer, lung cancer, and pancreatic cancer.


SEQUENCE LISTING (66 sequences)

SEQ ID NO: 1 - DNA, 20 bp, synthetic construct
  misc_feature 1..20: note = Emu_sgRNA
  source 1..20: mol_type = other DNA
gtctcaggagcggtgtctgt

SEQ ID NO: 2 - DNA, 20 bp, synthetic construct
  misc_feature 1..20: note = IGHG1_sgRNA_1; exon 2 (RJ178))
  source 1..20: mol_type = other DNA
tgagttttgtcacaagattt

SEQ ID NO: 3 - DNA, 20 bp, synthetic construct
  misc_feature 1..20: note = IGHG1_sgRNA_2; exon 2 (RJ180)
  source 1..20: mol_type = other DNA
gtgagttttgtcacaagatt

SEQ ID NO: 4 - DNA, 20 bp, synthetic construct
  misc_feature 1..20: note = IGHG1_sgRNA_3; exon 2 (RJ176)
  source 1..20: mol_type = other DNA
cacacatgcccaccgtgccc

SEQ ID NO: 5 - DNA, 20 bp, synthetic construct
  misc_feature 1..20: note = IGHG1_sgRNA_4; exon 3 (RJ182)
  source 1..20: mol_type = other DNA
caccacgcatgtgacctcag

SEQ ID NO: 6 - DNA, 20 bp, synthetic construct
  misc_feature 1..20: note = IGHG1_sgRNA_5; exon 3 (RJ183)
  source 1..20: mol_type = other DNA
ccaccacgcatgtgacctca

SEQ ID NO: 7 - DNA, 20 bp, synthetic construct
  misc_feature 1..20: note = IGHG1_sgRNA_6; exon 3 (RJ185)
  source 1..20: mol_type = other DNA
aggacaccctcatgatctcc

SEQ ID NO: 8 - DNA, 20 bp, synthetic construct
  misc_feature 1..20: note = IGHG1_sgRNA_7; exon 3 (RJ186)
  source 1..20: mol_type = other DNA
ggaagactgacggtcccccc

SEQ ID NO: 9 - DNA, 20 bp, synthetic construct
  misc_feature 1..20: note = IGHG1_sgRNA_8; exon 3 (RJ200)
  source 1..20: mol_type = other DNA
atggttttctcgatgggggc

SEQ ID NO: 10 - DNA, 20 bp, synthetic construct
  misc_feature 1..20: note = IGHG1_sgRNA_9; exon 3 (RJ201)
  source 1..20: mol_type = other DNA
gagcagtacaacagcacgta

SEQ ID NO: 11 - DNA, 20 bp, synthetic construct
  misc_feature 1..20: note = IGHG1_sgRNA_10; exon 1 (RJ173)
  source 1..20: mol_type = other DNA
gctcttggaggagggtgcca

SEQ ID NO: 12 - DNA, 20 bp, synthetic construct
  misc_feature 1..20: note = IGHG1_sgRNA_11; exon 1 (RJ174)
  source 1..20: mol_type = other DNA
gtgccagggggaagaccgat

SEQ ID NO: 13 - DNA, 20 bp, synthetic construct
  misc_feature 1..20: note = IGHG1_sgRNA_12; exon 4 (RJ191)
  source 1..20: mol_type = other DNA
gtgcagagcctcatgcatca

SEQ ID NO: 14 - DNA, 20 bp, synthetic construct
  misc_feature 1..20: note = IGHG1_sgRNA_13; exon 4 (RJ192)
  source 1..20: mol_type = other DNA
ctcatgctccgtgatgcatg

SEQ ID NO: 15 - DNA, 20 bp, synthetic construct
  misc_feature 1..20: note = IGHG1_sgRNA_14; exon 4 (RJ193)
  source 1..20: mol_type = other DNA
tcaccgtggacaagagcagg

SEQ ID NO: 16 - DNA, 20 bp, synthetic construct
  misc_feature 1..20: note = IGHG1_sgRNA_15; exon 4 (RJ194)
  source 1..20: mol_type = other DNA
tttggagatggttttctcga

SEQ ID NO: 17 - DNA, 20 bp, synthetic construct
  misc_feature 1..20: note = IGHG1_sgRNA_16; exon 4 (RJ195)
  source 1..20: mol_type = other DNA
agcctgacctgcctggtcaa

SEQ ID NO: 18 - DNA, 20 bp, synthetic construct
  misc_feature 1..20: note = IGHG1_sgRNA_17; exon 4 (RJ196)
  source 1..20: mol_type = other DNA
ggtcaggctgacctggttct

SEQ ID NO: 19 - DNA, 20 bp, synthetic construct
  misc_feature 1..20: note = IGHG1_sgRNA_18; exon 4 (RJ197)
  source 1..20: mol_type = other DNA
gtacaccctgcccccatccc

SEQ ID NO: 20 - DNA, 20 bp, synthetic construct
  misc_feature 1..20: note = IGHG1_sgRNA_19; exon 4 (RJ198)
  source 1..20: mol_type = other DNA
ccaggcaggtcaggctgacc

SEQ ID NO: 21 - DNA, 20 bp, synthetic construct
  misc_feature 1..20: note = IGHG1_sgRNA_20; exon 4 (RJ199)
  source 1..20: mol_type = other DNA
gggggcagggtgtacacctg

SEQ ID NO: 22 - DNA, 20 bp, synthetic construct
  misc_feature 1..20: note = CCR5_sgRNA; (RJ69)
  source 1..20: mol_type = other DNA
caatgtgtcaactcttgaca

SEQ ID NO: 23 - DNA, 20 bp, synthetic construct
  misc_feature 1..20: note = JCHAIN_sgRNA; (RJ94)
  source 1..20: mol_type = other DNA
aagaaccatttgcttttctg

SEQ ID NO: 24 - DNA, 20 bp, synthetic construct
  misc_feature 1..20: note = CD19_sgRNA_1; (RJ255)
  source 1..20: mol_type = other DNA
cctcgggcctgacttccatg

SEQ ID NO: 25 - DNA, 20 bp, synthetic construct
  misc_feature 1..20: note = CD19_sgRNA_2; RJ253
  source 1..20: mol_type = other DNA
cctcgggcctgacttccatg

SEQ ID NO: 26 - DNA, 20 bp, synthetic construct
  misc_feature 1..20: note = CD19_sgRNA_3; RJ254
  source 1..20: mol_type = other DNA
ggaacctctagtggtgaagg

SEQ ID NO: 27 - DNA, 20 bp, synthetic construct
  misc_feature 1..20: note = CD19_sgRNA_4; RJ256
  source 1..20: mol_type = other DNA
ttcaccactagaggttcctc

SEQ ID NO: 28 - DNA, 20 bp, synthetic construct
  misc_feature 1..20: note = IGHG1_sgRNA_10; exon 1 (sgYH_02)
  source 1..20: mol_type = other DNA
ttgtccaccttggtgttgct

SEQ ID NO: 29 - DNA, 450 bp, synthetic construct
  misc_feature 1..450: note = Emu_HA_L; Left arm for Emu_sgRNA
  source 1..450: mol_type = other DNA
tgtgacgcccggagacagaaggtctctgggtggctgggtttttgtggggtgaggatggacattctgccattgtgattactactactactactacatggacgtctggggcaaagggaccacggtcaccgtctcctcaggtaagaatggccactctagggcctttgttttctgctactgcctgtggggtttcctgagcattgcaggttggtcctcggggcatgttccgaggggacctgggcggactggccaggaggggatgggcactggggtgccttgaggatctgggagcctctgtggattttccgatgcctttggaaaatgggactcaggttgggtgcgtctgatggagtaactgagcctgggggcttggggagccacatttggacgagatgcctgaacaaaccaggggtcttagtgatggctgaggaatgtgtctcaggagcggtgtct

SEQ ID NO: 30 - DNA, 450 bp, synthetic construct
  misc_feature 1..450: note = Emu_HA_R; Right arm for Emu_sgRNA
  source 1..450: mol_type = other DNA
gtaggactgcaagatcgctgcacagcagcgaatcgtgaaatattttctttagaattatgaggtgcgctgtgtgtcaacctgcatcttaaattctttattggctggaaagagaactgtcggagtgggtgaatccagccaggagggacgcgtagccccggtcttgatgagagcagggttgggggcaggggtagcccagaaacggtggctgccgtcctgacaggggcttagggaggctccaggacctcagtgccttgaagctggtttccatgagaaaaggattgtttatcttaggaggcatgcttactgttaaaagacaggatatgtttgaagtggcttctgagaaaaatggttaagaaaattatgacttaaaaatgtgagagattttcaagtatattaatttttttaactgtccaagtatttgaaattcttatcatttgattaacacccatg

SEQ ID NO: 31 - DNA, 299 bp, synthetic construct
  misc_feature 1..299: note = CCR5_HA_L; Left arm for RJ69
  source 1..299: mol_type = other DNA
tcaagtgtcaagtccaatctatgacatcaattattatacatcggagccctgccaaaaaatcaatgtgaagcaaatcgcagcccgcctcctgcctccgctctactcactggtgttcatctttggttttgtgggcaacatgctggtcatcctcatcctgataaactgcaaaaggctgaagagcatgactgacatctacctgctcaacctggccatctctgacctgtttttccttcttactgtccccttctgggctcactatgctgccgcccagtgggactttggaaatacaatgtgtcaac

SEQ ID NO: 32 - DNA, 300 bp, synthetic construct
  misc_feature 1..300: note = CCR5_HA_R; Right arm for RJ69
  source 1..300: mol_type = other DNA
ctctattttataggcttcttctctggaatcttcttcatcatcctcctgacaatcgataggtacctggctgtcgtccatgctgtgtttgctttaaaagccaggacggtcacctttggggtggtgacaagtgtgatcacttgggtggtggctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccg

SEQ ID NO: 33 - DNA, 297 bp, synthetic construct
  misc_feature 1..297: note = IGHG1_HA_L; Left arm for RJ178 (TH26)
  source 1..297: mol_type = other DNA
tgggctctgcagagagaagattgggagttactggaatctgggaggagagaaggtgtccgagctgagggagtggagagtttggcctttggggtgggcttaggtcaggggcagggtcctcccggatatggctcttggcaggtctgagcccagcacctgcccctttgtgtgcagggcctgggttaggggcacctagcctgtgcctgcccagagcctggggaaaaagccagaagaccctctccctgagcatgagtggggcgggcagaggcctccgggtgaagaggcagacggggcctgcct

SEQ ID NO: 34 - DNA, 300 bp, synthetic construct
  misc_feature 1..300: note = IGHG1_HA_R; Right arm for RJ178 (TH26)
  source 1..300: mol_type = other DNA
atcttgtgacaaaactcacacatgcccaccgtgcccaggtaagccagcccaggcctcgccctccagctcaaggcgggacaggtgccctagagtagcctgcatccagggacaggccccagccgggtgctgacacgtccacctccatctcttcctcagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacg

SEQ ID NO: 35 - DNA, 297 bp, synthetic construct
  misc_feature 1..297: note = JCHAIN_HA_L; Left arm for RJ94
  source 1..297: mol_type = other DNA
tttatatagtgtaaatgcaaaataaaaatgattgtatcaaattatggtttttatttggatgctttgcaagaaaatacttaaatgtgagtcatacatttttccagttcagcatttaaacttcagacttcaaccacagttgtgattgtttttagtttgttagctgcctggagtgttattttaagaaagcagaagcaccatcatttgcacactccttatagatcacacaccttaaccctgactttttttgctccagtttttcagaagaagtgaagtcaagatgaagaaccatttgctttt

SEQ ID NO: 36 - DNA, 294 bp, synthetic construct
  misc_feature 1..294: note = JCHAIN_HA_R; Right arm for RJ94
  source 1..294: mol_type = other DNA
agtcctggcggtttttattaaggctgttcatgtgaaaggtatgtgatatttagaaaatgatcccagatcaaaggaaaaatataggacagatctgtttttcagttttaaacatttatgcctatgctttggttggccagttagccagctactttttcctaacatgctttcatctttctatggcagttgtctttacatagataagactctttttattcttttttctcttatcttacttacttgtttgcctactacaaactagagaataagaaattagctaactacagatagtcctgc

SEQ ID NO: 37 - DNA, 418 bp, synthetic construct
  misc_feature 1..418: note = CD19_HA_L; Left arm for RJ255
  source 1..418: mol_type = other DNA
caaataaacacacaagatcatttcccgtggtagtgagagctgggatgaaaataaaacagcgtggcagggaggaggcaagtgttgtgagtctggagggttcctggagaatggggcctgaggcgtgaccaccgccttcctctctggggggactgcctgccgcccccgcagacacccatggttgagtgccctccaggcccctgcctgccccagcatcccctgcgcgaagctgggtgccccggagagtctgaccaccatgccacctcctcgcctcctcttcttcctcctcttcctcacccccatcgaacagagaaacaggagaatatgggccaaacaggatatctgtggtaagcagttcctgccccggctcagggccaagaacagatggtccccagatgcggtcccgccctcagcagtttct

SEQ ID NO: 38 - DNA, 303 bp, synthetic construct
  misc_feature 1..303: note = CD19_HA_R; Right arm for RJ255
  source 1..303: mol_type = other DNA
ggaagtcaggcccgaggaacctctagtggtgaaggtggaaggtatgtccaaagggcagaaagggaagggattgaggctggaaacttgagttgtggctgggtgtccttggctgagtaacttaccctctctgagcctccattttcttatttgtaaaattcaggaaagggttggaaggactctgccggctcctccactcccagcttttggagtcctctgctctataacctggtgtgaggagtcggggggcttggaggtcccccccacccatgcccacacctctctccctctctctccacagaggga

SEQ ID NO: 39 - DNA, 400 bp, synthetic construct
  misc_feature 1..400: note = IGHG1_HA_L; left arm for TH16
  source 1..400: mol_type = other DNA
gcatgagccgtccctgaggtggcaccgatggccagagctgaggccaagctagaggccctggactgtgctgactcccggcagacacagagcgctgacctggctgccgagccccgcctcctaggctgcaggggtgcctgcagaagggcaccacagggccaccggtcctgcaagctttctggggcaggccgggcctgaccttggctttggggcagggggtgggctaaggtgacgcaggtggcgccagccaggcgcacacccaatgcccgtgagcccagacactggacgctgaacctcgcggacagttaagaacccaggggcctctgcgccctgggcccagctctgtcccacaccgcggtcacatggcaccacctctcttgcagcctccaccaagggcccatcg

SEQ ID NO: 40 - DNA, 400 bp, synthetic construct
  misc_feature 1..400: note = IGHG1_HA_R; right arm for TH16
  source 1..400: mol_type = other DNA
ggtgaagaggcagacggggcctgccttgctgccctggactggggctgcatagccgggatgcgtccaggcaggagcgctgagcctggcttccagcagacaccctccctccctgtgctggcctctcaccaactttcttgtccaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggcgcctgagttccacgacaccgtcaccggttcggggaagtagtccttgaccaggcagcccagggctgctgtgcccccagaggtgctcttggaggagggtgccagggggaagac

SEQ ID NO: 41 - DNA, 400 bp, synthetic construct
  misc_feature 1..400: note = IGHG1_HA_L; left arm for TH21
  source 1..400: mol_type = other DNA
aatggcaaggagtacaagtgcaaggtctccaacaaagccctcccagcccccatcgagaaaaccatctccaaagccaaaggtgggacccgtggggtgcgagggccacatggacagaggccggctcggcccaccctctgccctgagagtgaccgctgtaccaacctctgtccctacagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaaaagt

SEQ ID NO: 42 - DNA, 400 bp, synthetic construct
  misc_feature 1..400: note = IGHG1_HA_R; right arm for TH21
  source 1..400: mol_type = other DNA
agatggcagcaggggaacgtcttctcatgctccgtgatgcatgaggctctgcacaaccactacacacagaagagcctctccctgtctccgggtaaatgagtgccacggccggcaagcccccgctccccaggctctcggggtcgcgcgaggatgcttggcacgtaccccgtgtacatacttcccaggcacccagcatggaaataaagcacccagcgcttccctgggcccctgcgagactgtgatggttctttccacgggtcaggccgagtctgaggcctgagtggcatgagggaggcagagtgggtcccactgtccccacactggcccaggctgtgcaggtgtgcctgggccgcctagggtggggctcagccaggggctgccctcggcagggtgggggatt

SEQ ID NO: 43 - DNA, 400 bp, synthetic construct
  misc_feature 1..400: note = IGHG1_HA_L; left arm for TH22
  source 1..400: mol_type = other DNA
cgcacacccaatgcccgtgagcccagacactggacgctgaacctcgcggacagttaagaacccaggggcctctgcgccctgggcccagctctgtcccacaccgcggtcacatggcaccacctctcttgcagcctccaccaagggcccatcggtcttccccctggcaccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagt

SEQ ID NO: 44 - DNA, 400 bp, synthetic construct
  misc_feature 1..400: note = IGHG1_HA_R; right arm for TH22
  source 1..400: mol_type = other DNA
aatactaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgcccgccccactcatgctcagggagagggtcttctggctttttccccaggctctgggcaggcacaggctaggtgcccctaacccaggccctgcacacaaaggggcaggtgctgggctcagacctgccaagagccatatccgggaggaccctgcccctgacctaagcccaccccaaaggccaaactctccactccctcagctcggacaccttctctcctcccagattccagtaactccc

SEQ ID NO: 45 - DNA, 387 bp, synthetic construct
  misc_feature 1..387: note = IGHG1_HA_L; left arm for TH27
  source 1..387: mol_type = other DNA
ctgaggtggcaccgatggccagagctgaggccaagctagaggccctggactgtgctgactcccggcagacacagagcgctgacctggctgccgagccccgcctcctaggctgcaggggtgcctgcagaagggcaccacagggccaccggtcctgcaagctttctggggcaggccgggcctgaccttggctttggggcagggggtgggctaaggtgacgcaggtggcgccagccaggcgcacacccaatgcccgtgagcccagacactggacgctgaacctcgcggacagttaagaacccaggggcctctgcgccctgggcccagctctgtcccacaccgcggtcacatggcaccacctctcttgcagcctccaccaagggcccatcg

SEQ ID NO: 46 - DNA, 429 bp, synthetic construct
  misc_feature 1..429: note = IGHG1_HA_R; right arm for TH27
  source 1..429: mol_type = other DNA
caccctcctccaagagcacctctgggggcacagcagccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttggtgagaggccagcacagggagggagggtgtctgctggaagccaggctcagcgctcctgcctggacgcatcccggctatgcagccccagtccagggcagcaaggcaggccccgtctgcctcttcacccggaggcctctgccccgcggtctagagcatggctacgtagat

SEQ ID NO: 47 - DNA, 290 bp, synthetic construct
  misc_feature 1..290: note = IGHG1_HA_L; left arm for TH28
  source 1..290: mol_type = other DNA
ccgcctcctaggctgcaggggtgcctgcagaagggcaccacagggccaccggtcctgcaagctttctggggcaggccgggcctgaccttggctttggggcagggggtgggctaaggtgacgcaggtggcgccagccaggcgcacacccaatgcccgtgagcccagacactggacgctgaacctcgcggacagttaagaacccaggggcctctgcgccctgggcccagctctgtcccacaccgcggtcacatggcaccacctctcttgcagcctccaccaagggcccatcg

SEQ ID NO: 48 - DNA, 387 bp, synthetic construct
  misc_feature 1..387: note = IGHG1_HA_R; right arm for TH28
  source 1..387: mol_type = other DNA
ggtgaagaggcagacggggcctgccttgctgccctggactggggctgcatagccgggatgcgtccaggcaggagcgctgagcctggcttccagcagacaccctccctccctgtgctggcctctcaccaactttcttgtccaccttggtgttgctgggcttgtgattcacgttgcagatgtaggtctgggtgcccaagctgctggagggcacggtcaccacgctgctgagggagtagagtcctgaggactgtaggacagccgggaaggtgtgcacgccgctggtcagggcgcctgagttccacgacaccgtcaccggttcggggaagtagtccttgaccaggcagcccagggctgctgtgcccccagaggtgctcttggaggagggtg

SEQ ID NO: 49 - DNA, 1512 bp, synthetic construct
  misc_feature 1..1512: note = BTCE blinaumomab
  source 1..1512: mol_type = other DNA
gacattcagcttacgcagagccccgcgtctcttgcagtgagccttggtcaacgggcgacaatctcttgcaaggctagtcaatctgtagactatgacggagattcatatttgaactggtatcagcaaatcccgggccaaccgcctaaattgctcatatacgacgcatctaacctggtcagcgggattcccccgcgcttttccgggtccggttccgggacagatttcaccctgaacatccatcccgtcgaaaaggttgatgcggccacttaccattgccaacagtccactgaagacccctggacattcggtgggggaactaagctcgaaataaagagtggcgggggttccgggggtggcgggagtggtggtggcggaagtcaagtccaattgcagcaatccggtgctgaactcgtccgacccgggtcttccgtaaaaattagctgcaaggcgagcggctacgctttctcctcttattggatgaattgggttaagcagcggccaggacagggacttgagtggataggccaaatatggcctggggatggtgacacgaattacaacggtaagttcaaggggaaagctacactcacggcggacgagagttcaagcacggcgtatatgcagctctcatcactcgcatcagaagatagtgcagtttacttctgtgcgcgaagagagacaacaactgttgggcgctattactatgcgatggactattggggccaggggactacagttacggtctcatctgggggaggcggaagcgacataaagctacagcaaagtggtgcagagcttgcgcggccgggtgcctctgttaagatgagttgcaagacatccggatatactttcaccaggtacacaatgcattgggttaaacagagacccggacaggggttggaatggattggttacataaatccctccaggggttatactaactacaatcagaaattcaaagacaaagcaaccttgactacggacaaaagctcaagcactgcgtatatgcaactttcaagccttacaagcgaagattcagcggtgtactattgtgcacgctattacgacgatcactactgtctggactactgggggcaaggtactacgctgaccgtcagctccgtggaggggggttctggtgggagtggtggaagcggcggaagtggtggagtggacgatatacagttgacgcaaagcccagcgatcatgtctgcttctcctggagaaaaggttacgatgacatgcagagctagtagctctgtcagttatatgaattggtatcagcagaaaagcgggacgagtccgaaacggtggatatatgatacctccaaagtagcctctggagtaccgtaccgattttctggaagcggctcaggaactagctactcccttacgataagcagtatggaggcagaagacgctgccacatattactgtcaacagtggtccagcaacccccttacatttggtgctggtacgaagttggagttgaagcatcatcatcatcaccac

SEQ ID NO: 50 - DNA, 1536 bp, synthetic construct
  misc_feature 1..1536: note = BTCE AMG-330
  source 1..1536: mol_type = other DNA
atgcaggttcaactcgttcagagtggtgccgaggttaaaaaaccgggggaatccgtcaaagtaagctgcaaggcatccgggtatacgtttacgaactacgggatgaactgggtcaagcaagcgcccgggcaaggcttggaatggatgggctggatcaacacctacacaggtgagccgacctacgctgataagttccaaggacgagtaacgatgacaacggatacgtcaacatcaacggcctatatggagatacgaaacttgggtggggacgacacagcagtctactattgcgctaggtggagctggtctgatggctactacgtatacttcgattactggggtcaaggcacctccgtcacggtctcttccgggggtgggggttcaggaggagggggctccgggggtggtggatcagatattgtgatgactcagtccccagattcattgaccgtctcactgggagaacggactacgataaattgtaaatctagccaaagtgtacttgattcttcaacgaataaaaattctcttgcgtggtaccaacagaaacccggtcaacccccgaaactcctcttgtcctgggcgtccacacgggaatctggtatcccggatcggttcagtggctcaggatcagggacagattttactcttacaatcgatagtccccagcctgaggactcagcgacctactactgtcagcagtccgcgcactttccaattacgtttggccagggtacgaggctcgagatcaaaagcggaggtgggggtagcgaggtgcagttggtggagtctggaggcggcctcgttcaacccggtgggtcattgaaactttcatgtgctgcgtccggatttacttttaacaagtacgctatgaattgggtgcgacaagcaccgggtaaagggctggaatgggtggcacggataaggtctaaatacaataattatgctacgtactatgcagatagtgtaaaggataggttcacaataagtagggacgatagtaaaaatactgcgtatctgcagatgaataatttgaaaacagaggatacagcggtctactactgtgttaggcatgggaatttcggcaattcttacatatcttattgggcgtattggggacaaggcacacttgtaactgtcagtagcggtggaggtggtagcggaggcggcgggagtggaggtgggggatcacaaacggttgtgactcaggaaccaagccttacagtctctccaggaggcaccgtaacactgacttgcggctcaagtactggtgccgtaacttctggcaattatcctaactgggtccagcagaaaccaggtcaggcaccccggggactgataggcggcacaaagtttctggcaccgggcacccccgcacgcttttctggatcacttctcggaggcaaagcagccctcaccttgagcggcgtacaaccagaagacgaggctgaatactattgcgttctttggtacagtaaccgatgggttttcggagggggtacgaaactcacagtgcttcatcaccaccatcaccac

SEQ ID NO: 51 - DNA, 347 bp, synthetic construct
  misc_feature 1..347: note = Promoter MND
  source 1..347: mol_type = other DNA
gaacagagaaacaggagaatatgggccaaacaggatatctgtggtaagcagttcctgccccggctcagggccaagaacagttggaacagcagaatatgggccaaacaggatatctgtggtaagcagttcctgccccggctcagggccaagaacagatggtccccagatgcggtcccgccctcagcagtttctagagaaccatcagatgtttccagggtgccccaaggacctgaaatgaccctgtgccttatttgaactaaccaatcagttcgcttctcgcttctgttcgcgcgcttctgctccccgagctctatataagcagagctcgtttagtgaaccgtcagatc

SEQ ID NO: 52 - DNA, 300 bp, synthetic construct
  misc_feature 1..300: note = Promoter Igh1-69
  source 1..300: mol_type = other DNA
gtaatctttaggccaataaaatgtgggttcacagtgaggagtgcatcctggggttggggtttgttctgcagcgggaagagcgctgtgcacagaaagcttagaaatggggcaagagatgcttttcctcaggcaggatttagggcttggtctctcagcatcccacacttgtacagctgatgtggcatctgtgttttctttctcatcctagatcaggctttgagctgtgaaataccctgcctcatgcatatgcaaataacctgaggtcttctgagataaatatagatatattggtgccctgag

SEQ ID NO: 53 - DNA, 54 bp, synthetic construct
  misc_feature 1..54: note = Promoter minP
  source 1..54: mol_type = other DNA
actagagggtatataatggaagctcgacttccagcttggcaatccggtactgta

SEQ ID NO: 54 - DNA, 349 bp, synthetic construct
  misc_feature 1..349: note = Enhancer Emu
  source 1..349: mol_type = other DNA
atgcgggactgcgttttgaccatcataaatcaagtttatttttttaattaattgagcgaagctggaagcagatgatgaattagagtcaagatggctgcatgggggtctccggcacccacagcaggtggcaggaagcaggtcaccgcgagagtctattttaggaagcaaaaaaacacaattggtaaatttatcacttctggttgtgaagaggtggttttgcccaggcccagatctgaaagtgctctactgagcaaaacaacacctggacaatttgcgtttctaaaataaggcgaggctgaccgaaactgaaaaggctttttttaactatctgaatttcatttccaatctt

SEQ ID NO: 55 - DNA, 158 bp, synthetic construct
  misc_feature 1..158: note = Enhancer SLC3A2_5
  source 1..158: mol_type = other DNA
ggtccaattcacaagaaccagaaggatgatgtcgctcagactgacttgctgcagatcgaccccaattttggctccaaggaagattttgacagtctcttgcaatcggctaaaaaaaagagtgggtatcctggggttcccaaggaaacagctagaaagga

SEQ ID NO: 56 - DNA, 153 bp, synthetic construct
  misc_feature 1..153: note = Enhancer SLC3A2_3
  source 1..153: mol_type = other DNA
accgaggtggatatgaaggaggtggagctgaatgagttagagcccgagaagcagccgatgaacgcggcgtctggggcggccatgtccctggcgggagccgagaagaatggtctggtgaagatcaaggtggcggaagacgaggcggaggcggca

SEQ ID NO: 57 - DNA, 61 bp, synthetic construct
  misc_feature 1..61: note = 5' UTR Igh1-69_utr
  source 1..61: mol_type = other DNA
ggtgatgagctgtgctcccaggggcttcttcagaggaggaatgtggttgttatgtgatgct

SEQ ID NO: 58 - DNA, 143 bp, synthetic construct
  misc_feature 1..143: note = regulatory sequence_signal sequence Igh1-69_ex1_intron_ex2
  source 1..143: mol_type = other DNA
atggactggacctggaggttcctctttgtggtggcagcagctacaggtaaggggcttcctagtcctaaggctgaggaagggatcctggtttagttaaagaggattttattcacccctgtgtcctctccacaggtgtccagtcc

SEQ ID NO: 59 - DNA, 80 bp, synthetic construct
  misc_feature 1..80: note = 5' UTR Ighv3-49_utr
  source 1..80: mol_type = other DNA
agctctgggagaggagccccagccgtgagattcccaggagtttccacttggtgatcagcactgaacacagaccaccaacc

SEQ ID NO: 60 - DNA, 160 bp, synthetic construct
  misc_feature 1..160: note = regulatory sequence_signal sequence Ighv3-49_ex1_intron_ex2
  source 1..160: mol_type = other DNA
atggagtttgggcttagctgggttttccttgttgctattttaaaaggtaattcatggtgtactagagatactgagtgtgaggggacatgagtggtagaaacagtggatatgtgtggcagtttctgaccttggtgtttctgtgtttgcaggtgtccaatgt

SEQ ID NO: 61 - DNA, 135 bp, synthetic construct
  misc_feature 1..135: note = 3' UTR IGHG1_utr
  source 1..135: mol_type = other DNA
gtgccacggccggcaagcccccgctccccaggctctcggggtcgcgcgaggatgcttggcacgtaccccgtgtacatacttcccaggcacccagcatggaaataaagcacccagcgcttccctgggcccctgcga

SEQ ID NO: 62 - DNA, 135 bp, synthetic construct
  misc_feature 1..135: note = SV40 polyA
  source 1..135: mol_type = other DNA
tgctttatttgtgaaatttgtgatgctattgctttatttgtaaccattataagctgcaataaacaagttaacaacaacaattgcattcattttatgtttcaggttcagggggagatgtgggaggttttttaaagc

SEQ ID NO: 63 - DNA, 247 bp, synthetic construct
  misc_feature 1..247: note = WPRE
  source 1..247: mol_type = other DNA
ccacggaattgtcagtgcccaacagccgagcccctgtccagcagcgggcaaggcaggcggcgatgagttccgccgtggcaagaactaaccaggatttatacaaggaggagaaaatgaaagccatacgggaagcaatagcatgatacaaaggcattaaagcagcgtatccacatagcgtaaaaggagcaacatagttaagaataccagtcaatctttcacaaattttgtaatccagaggttgattatc

SEQ ID NO: 64 - DNA, 78 bp, synthetic construct
  misc_feature 1..78: note = P2A
  source 1..78: mol_type = other DNA
agggcaaagaggggatcaggagctacgaatttttctttattaaaacaagcaggagatgttgaggagaatcccggaccg

SEQ ID NO: 65 - DNA, 720 bp, synthetic construct
  misc_feature 1..720: note = Reporter GFP
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 66 - DNA, 78 bp, synthetic construct
  misc_feature 1..78: note = signal peptide
  source 1..78: mol_type = other DNA
atggctaccggcagcagaacaagcctgctgctcgcttttggactgctctgtctcccctggttgcaagaaggcagcgcc